(19) United States
(12) Patent Application Publication
(10) Pub. No.: US 2010/0168016 A1
(43) Pub. Date: Jul. 1, 2010

(54) DIAGNOSIS AND TREATMENT OF DISEASES CAUSED BY MISFOLDED PROTEINS

(75) Inventors: Susan Ackerman, Bar Harbor, ME (US); Jeong Wong Lee, Daejeon (KR)

Correspondence Address: DAVID S. RESNICK, NIXON PEABODY LLP, 100 SUMMER STREET, BOSTON, MA, 02110-2131, US

(73) Assignee: THE JACKSON LABORATORY, Bar Harbor, ME (US)

(21) Appl. No.: 12/593,988

(22) PCT Filed: Apr. 11, 2008
(86) PCT No.: PCT/US08/04757

§ 371 Date: Mar. 12, 2010

Related U.S. Application Data
(60) Provisional application No. 60/937,221, filed on Jun. 26, 2007.
(60) Provisional application No. 60/923,155, filed on Apr. 11, 2007.

(51) Int. Cl. A61K 38/17; A61K 31/7052; G01N 33/53; G01N 30/00; A61P 25/00; A61P 25/28; A61P 25/16

(52) U.S. Cl. 514/12; 514/44 R; 436/501; 422/69

(57) ABSTRACT

In certain aspects, the disclosure relates to methods and compositions for diagnosing and treating diseases caused by misfolded proteins that comprise monitoring ANKRD 16 expression and altering the activity of ANKRD 16 protein. In certain embodiments, the diseases are neurodegenerative diseases. In certain embodiments, the diseases are proteopathies. In certain embodiments, the disease is reduced fertility. Methods for identifying agents that protect against cell death are also provided.

### CROSS-REFERENCE TO RELATED APPLICATIONS

This Application claims the benefit under 35 U.S. §119(e) of U.S. Provisional Application No. 60/937,221 filed on Jun. 26, 2007, and of U.S. Provisional Application No. 60/923,155 filed on Apr. 11, 2007.

### FUNDING

This invention was made with government support under Grant Number NS042613, awarded by the National Institutes of Health. The government has certain rights in the invention.

### BACKGROUND OF THE INVENTION

Degenerative diseases affect many, particularly in the aging population; however the molecular mechanisms underlying the pathogenesis of these disorders are poorly understood. Many of the degenerative diseases show aberrant polymerization and accumulation of specific proteins. Such disorders can be summarized as proteopathies (Walker et al., 2006). Folding of linear peptide chains into biologically active, three-dimensional proteins must occur for all newly synthesized proteins. If folding does not occur properly, hydrophobic residues that are usually buried in the interior of proteins may be exposed, leading to inappropriate molecular interactions and abnormal aggregation.

### SUMMARY OF THE INVENTION

Embodiments of the present invention are based on the discovery that ANKRD16 protein is a regulator of protein degradation pathways capable of removing misfolded proteins from cells.

In certain aspects, the disclosure relates to an ANKRD16 protein comprising SEQ ID No: 14, wherein the protein consists of less than the full-length sequence. In certain embodiments, said protein is 90% identical to SEQ ID No: 4. In certain embodiments, said protein is 90% identical to SEQ ID No: 6. In certain embodiments, said protein comprises SEQ ID No: 8. In certain embodiments, said protein comprises SEQ ID No: 23, 24, or a longer sequence comprising the unique splice junction of ANKRD16 isoform B. In certain embodiments, said protein comprises SEQ ID No:10.

In certain aspects, the disclosure relates to an ANKRD 16 protein comprising SEQ ID No: 8. In certain embodiments, said protein comprises SEQ ID No: 23, 24, or a longer sequence comprising the unique splice junction of ANKRD16 isoform B.

In certain aspects, the disclosure relates to an ANKRD16 protein comprising SEQ ID No: 10.

In certain aspects, the disclosure relates to a nucleic acid encoding an ANKRD16 protein according to any of the proceeding embodiments.

In certain aspects, the disclosure relates to a nucleic acid encoding an ANKRD16 protein that expresses higher levels of full-length ANKRD16 protein than shorter isoforms. In certain embodiments, the nucleic acid is further comprising a vector that expresses the protein in cells. In certain embodiments, the cells are mammalian, plant, insect or prokaryotic.

In certain aspects, the disclosure relates to transgenic animals that express the ANKRD16 isoforms of the invention.

In certain aspects, the disclosure relates to genetically engineered animals that carry a mutation leading to a loss of function of one or both alleles of the ANKRD 16 gene of the invention.

In certain aspects, the disclosure relates to an antibody or antigen binding fragment thereof that binds ANKRD 16 protein. In certain embodiments, said antibody binds specifically to ANKRD 16 protein. In certain embodiments, said antibody binds SEQ ID No: 8. In certain embodiments, said antibody binds SEQ ID No: 23, 24, or a longer sequence comprising the unique splice junction of ANKRD16 isoform B. In certain embodiments, said antibody binds SEQ ID No: 10. In certain embodiments, said antibody binds SEQ ID No: 12. In certain embodiments, said antibody binds SEQ ID No: 25, 26, or a longer sequence comprising the unique sequence of ANKRD16 isoform A. In certain embodiments, said antibody binds SEQ ID No: 2 with higher affinity than SEQ ID No: 4 or SEQ ID No: 6. In certain embodiments, said antibody binds SEQ ID No: 4 with higher affinity than SEQ ID No: 2 or SEQ ID No: 6. In certain embodiments, said antibody binds SEQ ID No: 6 with higher affinity than SEQ ID No: 2 or SEQ ID No: 4. In certain embodiments, said antibody or antigen binding fragment thereof is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fd, an Fab, an Fab′, and an F(ab′)2. In certain embodiments, said antibody or antigen binding fragment thereof is a monoclonal antibody. In certain embodiments, said monoclonal antibody is a humanized antibody. In certain embodiments, said antibody or antigen binding fragment thereof is a polyclonal antibody. In certain embodiments, said antibody or antigen binding fragment thereof is covalently linked to an additional functional moiety. In certain embodiments, the additional functional moiety is a detectable label. In certain embodiments, the detectable label is selected from a fluorescent or chromogenic label. In certain embodiments, said detectable label is selected from horseradish peroxidase or alkaline phosphatase. In certain embodiments, the antibody or antigen binding fragment thereof is an agonistic antibody.

In certain aspects, the disclosure relates to a non-immunoglobulin antigen-binding scaffold that binds ANKRD 16 protein. In certain embodiments, the scaffold is selected from the group consisting of: an antibody substructure, minibody, adnectin, anticalin, affibody, affilin, knottin, glubody, C-type lectin-like domain protein, designed ankyrin-repeate proteins (DARPin), tetranectin, kunitz domain protein, thioredoxin, cytochrome b562, zinc finger scaffold, Staphylococcal nuclease scaffold, fibronectin or fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VCAM-1,1-set immunoglobulin domain of myosin-binding protein C, 1-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin.

In certain aspects, the disclosure relates to a non-immunoglobulin antigen-binding scaffold that binds ANKRD16 according to any one of the previous embodiments.

In certain aspects, the disclosure relates to a nucleic acid that comprises a region that binds to any one of SEQ ID Nos: 7, 9, or 11, wherein said nucleic acid consists of at least 18 nucleotides. In certain embodiments, said nucleic acid consists of at least 19, 20, 21, 22, 23, 24, or 25 nucleotides. In certain embodiments, said nucleic acid consists of approximately 30, 40, 50, 60, 70, 80, 90 or 100 nucleotides.

In certain aspects, the disclosure relates to nucleic acid that comprises a region that binds to a nucleic acid according to any one of the previous embodiments.

In certain aspects, the disclosure relates to a nucleic acid that binds to SEQ ID No: 3 with higher affinity than to SEQ ID No: 1 or SEQ ID No: 5.

In certain aspects, the disclosure relates to a nucleic acid that binds to SEQ ID No: 5 with higher affinity than to SEQ ID No: 1 or SEQ ID No: 3.

In certain aspects, the disclosure relates to a method of treating a neurodegenerative disease, the method comprising administering to a subject in need thereof an effective amount of a composition comprising ANKRD16 protein.

In certain aspects, the disclosure relates to a method of treating a neurodegenerative disease, the method comprising administering to a subject in need thereof an effective amount of a composition comprising a nucleic acid encoding an ANKRD 16 protein.

In certain aspects, the disclosure relates to a method of treating a neurodegenerative disease, the method comprising administering to a subject in need thereof an effective amount of a composition comprising cells expressing ANKRD16.

In certain aspects, the disclosure relates to a method of treating a neurodegenerative disease, the method comprising administering to a subject in need thereof an effective amount of a composition comprising an ANKRD16 activator.

In certain aspects, the disclosure relates to a method of treating a disease caused by protein misfolding, the method comprising administering to a subject in need thereof an effective amount of a composition comprising ANKRD16 protein.

In certain aspects, the disclosure relates to a method of treating a disease caused by protein misfolding, the method comprising administering to a subject in need thereof an effective amount of a composition comprising a nucleic acid encoding ANKRD16.

In certain aspects, the disclosure relates to a method of treating a disease caused by protein misfolding, the method comprising administering to a subject in need thereof an effective amount of a composition comprising cells expressing ANKRD16.

In certain aspects, the disclosure relates to a method of treating a disease caused by protein misfolding, the method comprising administering to a subject in need thereof an effective amount of a composition comprising an ANKRD 16 activator.

In certain embodiments of the above methods, ANKRD16 protein is selected from SEQ ID Nos: 2, 4, 6, 8, 10, 12, 14 or 22. In certain embodiments, a neurodegenerative disease is selected from the group consisting of: Alexander disease, Alper's disease, Alzheimer's disease, Amyotrophic lateral sclerosis, Ataxia telangiectasia, Batten disease, Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease, Multiple sclerosis, Multiple System Atrophy, Parkinson disease, Pelizaeus-Merzbacher Disease, Pick's disease, Primary lateral sclerosis, Prion diseases, Refsum's disease, Sandhoff disease, Schilder's disease, Schizophrenia, Spielmeyer-Vogt-Sjogren-Batten disease, Spinocerebellar ataxia, Spinal muscular atrophy, Steele-Richardson-Olszewski disease, or Tabes dorsalis. In certain embodiments, said ANKRD16 activator is selected from the group consisting of a polypeptide, a polypeptide analog, a peptidomimetic, an antibody, a nucleic acid, an RNAi construct, microRNA, short hairpin RNA, a nucleic acid analog, a non-immunoglobulin antigen-binding scaffold, or a small molecule (including prodrugs). In certain embodiments, said ANKRD16 activator is an RNAi construct (including siRNA molecules) targeting an ANKRD16 inhibitor. In certain embodiments, said composition is administered systemically. In certain embodiments, said composition is administered locally. In certain embodiments, said subject is a human. In certain embodiments, said subject is another type of mammalian subjects such as a dogs or cat. In certain embodiments, said composition promotes cell survival. In certain embodiments, said composition is formulated with a pharmaceutically acceptable carrier. In certain embodiments, the method further comprises at least one additional therapeutic for a neurodegenerative disease. In certain embodiments, said therapeutic for a neurodegenerative disease and said composition are administered serially. In certain embodiments, said therapeutic for a neurodegenerative disease and said composition are administered simultaneously.

In certain embodiments of the above methods, ANKRD16 protein is selected from SEQ ID Nos: 2, 4, 6, 8, 10, 12, 14 or 22. In certain embodiments, a proteopathy is selected from the group consisting of infertility, reduced fertility, cancer, Hereditary lattice corneal dystrophy, cataracts, myopathy, amyloidosis, diabetes, medullary thyroid carcinoma, Pituitary prolactinoma, and Pulmonary alveolar proteinosis. Amyloidosis includes AL (light chain) amyloidosis (primary systemic amyloidosis), AA (secondary) amyloidosis, Aortic medial amyloidosis, ApoAI amyloidosis, ApoAII amyloidosis, ApoAIV amyloidosis, Finnish hereditary amyloidosis, Lysozyme amyloidosis, Fibrinogen amyloidosis, Cardiac atrial amyloidosis, Cutaneous lichen amyloidosis, Corneal lactoferrin amyloidosis, etc.

In certain aspects, the disclosure relates to a method of detecting whether a subject has or is at risk of developing a neurodegenerative disease comprising:

(a) contacting a sample obtained from said subject with at least one probe that binds at least one ANKRD16 isoform; and

(b) assessing the presence of full-length and short ANKRD 16 isoforms, wherein the presence of higher amounts of short ANKRD16 isoforms compared to the full-length ANKRD16 isoform is indicative that the subject has or is at risk of developing a neurodegenerative disease.

In certain aspects, the disclosure relates to a method of developing a prognosis for a subject suffering from a neurodegenerative disease comprising:

(a) contacting a sample obtained from said subject with at least one probe that binds at least one ANKRD16 isoform; and

(b) assessing the presence of full-length and short ANKRD 16 isoforms, wherein the presence of higher amounts of short ANKRD16 isoforms compared to the full-length ANKRD 16 isoform is indicative of a poor prognosis.

In certain embodiments of the above methods, said probe is selected from the group consisting of an antibody or antigen binding fragment thereof or a nucleic acid. In certain embodiments, said isoform is selected from the group consisting of protein or RNA isoforms. In certain embodiments, said subject is a human. In certain embodiments, said subject is another type of mammalian subjects such as a dogs or cat. In certain embodiments, said neurodegenerative disease is selected from the group consisting of: Alexander disease, Alper's disease, Alzheimer's disease, Amyotrophic lateral sclerosis, Ataxia telangiectasia, Batten disease, Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease, Multiple sclerosis, Multiple System Atrophy, Parkinson disease, Pelizaeus-Merzbacher Disease, Pick's disease, Primary lateral sclerosis, Prion diseases, Refsum's disease, Sandhoff disease, Schilder's disease, Schizophrenia, Spielmeyer-Vogt-Sjogren-Batten disease, Spinocerebellar ataxia, Spinal muscular atrophy, Steele-Richardson-Olszewski disease, or Tabes dorsalis. In certain embodiments, said antibody or antigen binding fragment thereof is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fd, an Fab, an Fab′, and an F(ab′)2. In certain embodiments, said antibody or antigen binding fragment thereof is a polyclonal antibody. In certain embodiments, a sample is selected from the group consisting of: a tissue sample, a blood sample, a cerebrospinal fluid sample, a saliva sample, or a serum sample. In certain embodiments, the antibody or antigen binding fragment thereof is covalently linked to an additional functional moiety. In certain embodiments, the additional functional moiety is a detectable label. In certain embodiments, the detectable label is selected from a fluorescent or chromogenic label. In certain embodiments, said detectable label is selected from horseradish peroxidase or alkaline phosphatase.

In certain aspects, the disclosure relates to a kit for diagnosing a neurodegenerative disease comprising at least one probe that binds at least one ANKRD16 isoform, a detectable label, and instructions for using the kit. In certain embodiments, said probe is selected from the group consisting of an antibody or antigen binding fragment thereof or a nucleic acid. In certain embodiments, said isoform is selected from the group consisting of protein or RNA isoforms. In certain embodiments, the detectable label is fluorescent or chromogenic. In certain embodiments, said detectable label comprises horseradish peroxidase or alkaline phosphatase.

In certain aspects, the disclosure relates to a method of expressing a recombinant ANKRD16 protein comprising:

(a) providing a nucleic acid encoding said recombinant protein and cells capable of expressing said protein;

(b) introducing said nucleic acid into said cells; and

(c) expressing said recombinant protein.

In certain embodiments, the cell are selected from the group consisting of: E. coli cells, Bacillus cells, Caulobacter cells, yeast cells (e.g. Pichia pastoris, Saccharomyces cerevisiae, Schizosaccharomyces pombe) insect cells (e.g., baculovirus, Sf9 Sf21 cells), mammalian cells (e.g., CHO, COS, NIH 3T3, BHK, HEK, 293,L929, MEL, JEG-3), algae (e.g. Chlamydomonas reinhardtii) or plant (e.g. tobacco, potato, pea). In certain embodiments, the ANKRD 16 protein is selected from the group consisting of SEQ ID Nos: 2, 4, 6, 8, 10, 12, 14 or 22.

Recombinant protein can also be expressed in vitro, using a cell-free system. Exemplary cell-free expression system includes, for example, Expressway™ Cell-Free Expression System by Invitrogen. (Invitrogen, cat. no. K9900-96) or Rapid Translation System (RTS) by Roche (e.g. RTS 500 E. coli HY kit, cat. no. 3 246 817)

In certain aspects, the disclosure relates to a method of expressing a recombinant ANKRD16 protein, wherein the recombinant protein comprises a signaling sequence for cell penetration (CPP peptides), or activated CPP peptides, allowing intracellular delivery comprising:

(a) providing a nucleic acid encoding said recombinant protein and cells capable of expressing said protein;

(b) introducing said nucleic acid into said cells; and

(c) expressing said recombinant protein.

CPP peptides are short cationic peptide sequences capable to mediate intracellular transport. Examples for CPPs include antennapedia, TAT, transportan and polyarginine and can be further modified that they are active on specific sites (see, e.g., Jones et al. British Journal of Pharmacology (2005) 145, 1093-1102, WO2006125134, Jiang et al., Proc. Natl. Acad. Sci. USA, (2004), 101, 17867-17872).

In certain aspects, the disclosure relates to an in vitro assay for identifying agents that protect against cell death comprising:

(a) providing sticky mutant cells;

(b) contacting cells in culture with an agent;

(c) assaying cell death in increasing concentrations of a non-cognate amino acid; and

(d) comparing the results to control treated cells, wherein an agent that decreases cell death protects against cell death. In certain embodiments, the cells are mouse embryonic fibroblasts. In certain embodiments, the non-cognate amino acid is serine.

In certain aspects, the disclosure relates to the use of an ANKRD 16 composition in the manufacture of a medicament for the treatment of a neurodegenerative disease. In certain embodiments, the ANKRD16 composition comprises ANKRD16 protein, a peptide, a nucleic acid encoding ANKRD16, a cell composition expressing ANKRD16 or an ANKRD16 activator. In certain embodiments, the ANKRD 16 activator is selected from the group consisting of a polypeptide, a polypeptide analog, a peptidomimetic, an antibody, a nucleic acid, an RNAi construct, a microRNA, a shRNA, a nucleic acid analog, a non-immunoglobulin antigen-binding scaffold, or a small molecule. In certain embodiments, the ANKRD16 protein is selected from SEQ ID Nos: 2, 4, 6, 8, 10, 12, 14 or 22. In certain embodiments, a neurodegenerative disease is selected from the group consisting of: Alexander disease, Alper's disease, Alzheimer's disease, Amyotrophic lateral sclerosis, Ataxia telangiectasia, Batten disease, Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease, Multiple sclerosis, Multiple System Atrophy, Parkinson disease, Pelizaeus-Merzbacher Disease, Pick's disease, Primary lateral sclerosis, Prion diseases, Refsum's disease, Sandhoff disease, Schilder's disease, Schizophrenia, Spielmeyer-Vogt-Sjogren-Batten disease, Spinocerebellar ataxia, Spinal muscular atrophy, Steele-Richardson-Olszewski disease, or Tabes dorsalis.

In certain aspects, the disclosure relates to a method of detecting whether a subject has or is at risk of developing a neurodegenerative disease or a proteopathy, comprising:

(a) assessing the level ANKRD16 in a test sample obtained from the subject; and

(b) comparing the level determined in step (a) with a control level of ANKRD16;

wherein the presence of lower amounts of ANKRD 16 in the test sample as compared to the control level is indicative that the subject has or is at risk of developing a neurodegenerative disease or a proteopathy.

In certain aspects, the disclosure relates to a method of developing a prognosis for a subject suffering from a neurodegenerative disease or a proteopathy, comprising:

(a) assessing the level ANKRD 16 in a test sample obtained from the subject; and

(b) comparing the level determined in step (a) with a control level of ANKRD16;

wherein the presence of lower amounts of ANKRD16 in the test sample as compared to the control level is indicative of a poor prognosis.

In certain embodiments, of the above methods, the level of ANKRD16 is assessed by measuring the level of ANKRD16 protein. In other embodiments, the level of ANKRD16 is assessed by measuring the level of ANKRD16 mRNA. In certain embodiments, said subject is a human. In certain embodiments, the neurodegenerative disease is selected from the group consisting of: Alexander disease, Alper's disease, Alzheimer's disease, Amyotrophic lateral sclerosis, Ataxia telangiectasia, Batten disease, Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease, Multiple sclerosis, Multiple System Atrophy, Parkinson disease, Pelizaeus-Merzbacher Disease, Pick's disease, Primary lateral sclerosis, Prion diseases, Refsum's disease, Sandhoff disease, Schilder's disease, Schizophrenia, Spielmeyer-Vogt-Sjogren-Batten disease, Spinocerebellar ataxia, Spinal muscular atrophy, Steele-Richardson-Olszewski disease, or Tabes dorsalis. In certain embodiments, the proteopathy is selected from the group consisting of: infertility, reduced fertility, cancer, Hereditary lattice corneal dystrophy, cataracts, myopathy, amyloidosis, diabetes, medullary thyroid carcinoma, Pituitary prolactinoma, and Pulmonary alveolar proteinosis.

In certain embodiments, the level of ANKRD16 protein is measured by a method comprising the steps of: (a) contacting the test sample, or preparation thereof, with an antibody or antigen binding fragment or non-immunoglobulin binding protein, which specifically binds ANKRD16 protein to form an antibody or antigen binding-ANKRD16 protein complex; and (b) detecting the presence of the complex, thereby measuring the level of ANKRD16 protein. In certain embodiments the antibody or antigen binding fragment thereof is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fd, an Fab, an Fab′, and an F(ab′)2. In certain embodiments the non-immunoglobulin binding protein is selected from the group consisting of antibody substructure (e.g. Fc fragment), minibody, adnectin, anticalin, affibody, affilin, DARPin, knottin, glubody, C-type lectin-like domain protein, tetranectin, kunitz domain protein, thioredoxin, cytochrome b562, zinc finger scaffold, Staphylococcal nuclease scaffold, fibronectin or fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VCAM-1,1-set immunoglobulin domain of myosin-binding protein C, 1-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin. In certain embodiments, the test sample is selected from the group consisting of: a tissue sample, a blood sample, a cerebrospinal fluid sample, a saliva sample, or a serum sample. In certain embodiments, the antibody or antigen binding fragment thereof is crosslinked with a detectable label. In certain embodiments, the detectable label is selected from a fluorescent or chromogenic label. In certain embodiments, the detectable label is selected from horseradish peroxidase or alkaline phosphatase.

The application contemplates combinations of any of the foregoing aspects and embodiments.

### BRIEF DESCRIPTION OF THE DRAWINGS

FIG. 1. A diagram illustrating the ubiquitin-proteasome system. Most proteins targeted for degradation by the proteasome are modified by ubiquitin via ubiquitin-activating (E1) enzymes and ubiquitin-conjugating (E2) and ubiquitin ligase (E3) enzymes. Poly-ubiquitinated proteins are recognized by proteins usually containing a UBA domain and shuttled to the proteasome where they are recognized by proteins including ATPase/AAA proteins in the base of the RP.

FIG. 2. A schematic illustrating alternative splicing of the ANKRD16 transcript in C57BL/6J (B6), Balb/cJ, C3H/HeJ and DBA/2J. In the mouse strains C57BL/6J (B6), Balb/cJ, C3H/HeJ, DBA/2J and MOLF/J the ANKRD16 mRNA is alternatively spliced when compared to CAST/EiJ and CASA/RkJ mRNA. Intron sequence is in lower case, exon sequence in upper case. The underline indicates the splice acceptor and splice donor in exon 5. Amino acid polymorphisms encoded by exon 7 are shown.

FIG. 3. A schematic illustrating ANKRD16 protein isoforms in different mouse strains.

FIG. 4. A schematic illustrating genes in the ANKRD16 critical region. Polymorphic genes (open arrows) and polymorphic amino acids are indicated. The BAC used for transgenic mouse generation is shown.

FIG. 5. A graph depicting viability curves for mouse embryonic fibroblasts (MEFs) with increasing concentrations of serine in the culture media. ANKRD 16 (Stim^{CAST}) rescues serine-mediated cell death in sti/sti fibroblasts. Values are the means of three independent experiments ±S.E.M. The heterogeneity of slope associated with concentration for each genotype was determined by ANCOVA analysis. *=p<0.001, sti/sti is significantly different from both Stim^{CAST}; sti/sti and wild type. **=p>0.4; no significant difference between Stim; sti/sti and wild type.

FIG. 6. A schematic illustrating the ANKRD16 transgene driven by Pcp2 Purkinje cell-specific promoter.

FIGS. 7. A graph depicting litter size (y axis) in WT, STIM and STI mice (x axis). Expression of ANKRD16 protein can restore the reduced fertility in sticky mutant mice. “WT” refers to C57BL/6J wild type mice, “STIM” refers to sticky mutant mice carrying the modifier gene ANKRD16, and STI means homozygous sticky mutant mice.

FIG. 8. A SDS PAGE gel showing the recombinant expression of ANKRD16 protein in Escherichia coli. In lane 1 the unpurified Escherichia coli lysate is shown. Lane 2 shows the expression of the full lengths ANKRD 16 with a GST tag after a one-step Glutathione-Sepharose purification. The arrow indicates the full length GST-tagged ANKRD 16 recombinant protein.

FIGS. 9A and B. Graphs illustrating that the expression of full length ANKRD16 reduces protein inclusions in sti/sti Purkinje cells. Sti/sti means homozygous sticky mice (B6.Cg-Aarssti/J). ANKRD16; sti/sti means homozygous sticky mice intercrossed with mice expressing full length ANKRD16. Graph A shows the total number of Purkinje cells with protein aggregates (inclusions) and the Graph B shows the percentage of Purkinje cells with protein aggregates (inclusions). 4W means brains were isolated from 4 week old mice, 6W for 6 week old mice and 12 W for 12 week old mice.

FIG. 10. Shows a Western blot with protein extracts from the cerebellum of CAST/EiJ (CAST), C57BL/6J (B6) and ANKRD16 deficient (−/−) mice. The upper band unspecific cross-reaction with the polyclonal antibody against ANKRD16. The lower band shows the ANKRD16 protein, with highest levels in CAST/EiJ and no detectable protein levels in ANKRD 16-null mice.

FIG. 11. Shows a schematic diagram of the genomic locus for the AnkRD16 gene and diagrams of the targeted allele before and after Cre-mediated excision of genomic DNA.

FIG. 12. Shows a Western blot with samples derived from C57BL/6J wild type (WT), UbG76V-GFP transgenic and UbG76V-GFP; ANKRD16^{CAST} double transgenic mice. In the upper panel the expression of ANKRD16 protein was analyzed using the rabbit polyclonal ANKRD 16 antibody. The lower panel shows beta-tubulin expression for normalization.

FIG. 13. Shows a graph depicting the mean GFP intensity after FACS analysis. Mouse embryonic fibroblasts were analyzed from UbG76V-GFP transgenic and UbG76V-GFP; ANKRD16^{CAST} double transgenic mice; untreated and treated with 1000 nM epoximicin for 6; 12 or 18 hours (hr). The presence of the ANKRD16^{CAST} allele leads to reduced GFP intensity which correlates with increased proteasome activity.

FIG. 14. Shows a graph illustrating the percentage of cell death as determined by FACS analysis of mouse embryonic fibroblasts, untreated and treated with 500, 1000, 1500, 2000 or 2500 nM epoximicin. Fibroblasts with different genotypes were compared: WT means C57BL/6J; UbG76VGFP transgenic and ANKRD16^{CAST} UbG76V-GFP double transgenic.

FIG. 15. Shows sagittal sections of the cerebellum from homozygous sticky (sti/sti) mice and from mice being homozygous for sticky and heterozygous for ANKRD16 (sti/sti; ankrd16+/−) at the 3 weeks and 9 weeks of age. The cerebellum sections are stained with an antibody to calbindin D-28, which reveals less neurons in the cerebellum of mice lacking one copy of ANKRD16, thus having a lower expression of ANKRD16.

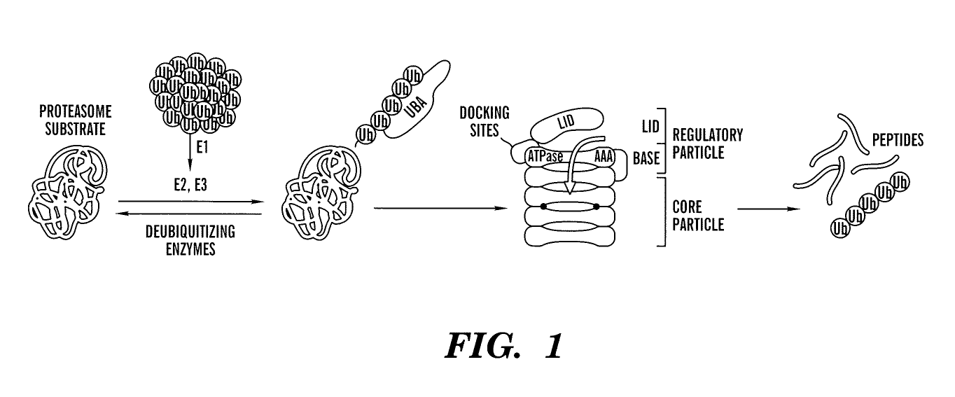

### DETAILED DESCRIPTION OF THE INVENTION

### I. Overview

Oligomerization and the formation of aggregates of misfolded proteins are common to many genetic and sporadic forms of these diseases, even though the specificity of various disorders may differ. In the case of neuronal degenerative diseases accumulations of abnormal proteins are associated with neuronal disturbances that include cytoskeletal and axonal transport defects, and synaptic dysfunction. Some of these misfolded proteins are due to mutations directly within disease-related proteins, such as in the polyglutamine expansion diseases and some forms of familial Alzheimer's disease (AD). However, the mechanisms underlying protein misfolding in many neurodegenerative diseases remain unknown. Abnormal protein aggregates are a common pathology associated with many adult-onset diseases: Huntington's disease (HD), Parkinson's disease (PD), amyotrophic lateral sclerosis (ALS), spinocerebellar ataxia, tauopathy, frontotemporal dementias, ataxia, ischemia (stroke), Creutzfeldt—Jakob disease and prion disease. Proteopathies can also affect other organs, tissues and cells like in primary systemic amyloidoses (Amyloid A and ApoAII amyloidoses), type II diabetes, cystic fibrosis and cancer (Cohen and Kelly, 2003; Gatchel and Zoghbi, 2005; Giffard et al., 2004; Gregersen et al., 2005; Kopito and Ron, 2000; Ross and Poirier, 2004, 2005; Selkoe, 2003; Taylor et al., 2002). Accumulation of fibrillar aggregates of misfolded proteins in these diseases may occur intracellularly (as seen with hyperphosphorylated tau in the tauopathies, repeat expansion proteins in the polyglutamine-expansion diseases and Lewy bodies in PD) or extracellular (e.g., amyloid plaques in AD).

Misfolded protein aggregates have been associated with many cellular and molecular pathogenic phenomena including loss of protein activity or abundance, abnormal protein or RNA-protein interactions, synaptic dysfunction, defective axonal transport, oxidative stress and ER stress (Bossy-Wetzel et al., 2004; Gatchel and Zoghbi, 2005). However, recent evidence suggests that inclusion body or amyloid fibril formation is likely an end-stage process, representing a protective mechanism to sequester these misfolded proteins within the cell (Caughey and Lansbury Jr, 2003; Ross and Poirier, 2005). For example, while the identification of mutations within the gene that encodes the aggregated protein, as seen in the dominant polyglutamine expansion diseases, or in genes that regulate the levels of the misfolded protein (i.e., presenillin mutations in early AD) demonstrate that abnormal conformation of these disease-associated proteins causes disease, there is often only a weak correlation between aggregate size/number (i.e., density of amyloid-beta plaques, Lewy bodies, or HD inclusions) and the clinical or pathological severity of the disease (Gutekunst et al., 1999; Kuemmerle et al., 1999; Saudou et al., 1998; Terry et al., 1991; Tompkins and Hill, 1997). Data from in vitro experiments have also often failed to demonstrate a correlation between aggregate formation and cell death. For example, inclusion size or number does not predict cell death in cells overexpressing an N-terminal huntingtin fragment or mutant alpha-synuclein (Arrasate et al., 2004; Petrucelli et al., 2002; Stefanis et al., 2001; Tanaka et al., 2004; Tanaka et al., 2001). In fact cells with inclusion bodies often survived better than those without, in agreement with previous suggestions that inclusion body formation is a protective response. Together these studies suggest that while terminally differentiated neurons are highly susceptible to misfolded proteins, it is likely that toxicity results from abnormally folded intermediates rather than highly ordered inclusion bodies.

### Chaperone Modulation of Degeneration.

The cell has several defense mechanisms to deal with unfolded proteins, including refolding of these proteins via molecular chaperones. Molecular chaperones are often co-localized with extracellular plaques and intracellular inclusions in human post-mortem tissue (Banal et al., 2004; Muchowski, 2002; Muchowski and Wacker, 2005; Sherman and Goldberg, 2001). These molecules enhance proper folding of newly synthesized proteins, and help prevent misfolding by binding in an ATP-dependent fashion to exposed hydrophobic regions and preventing inappropriate intra- and intermolecular interactions (Homma et al., 2006; Widmer et al., 2004). In addition, these proteins are often upregulated by cellular stress pathways to aid in the refolding of misfolded proteins that accumulate within the cell (Lindquist, 1986).

Molecular chaperones are characterized into families by the approximate molecular mass (Hartl and Hayer-Hartl, 2002; Mayer and Bukau, 2005). Members of the Hsp70 family are among the first proteins to bind newly synthesized polypeptides to aid in folding and transport across the intracellular membranes (Fink, 1999; Mayer et al., 2003). These proteins are found in cellular compartments including the cytosol (inducible Hsp70 and the constitutive Hsc70) and the endoplasmic reticulum (BiP, also called GRP78). In an ATP-bound conformation the peptide binding pocket of the Hsp70 is open, causing rapid peptide binding and release but low binding affinity. ATP hydrolysis converts Hsp70 to an ADP-bound conformation with high substrate affinity. The ATP/ADP state of Hsp70 chaperones is regulated by two classes of co-chaperones(Muchowski and Wacker, 2005). Members of the DNAJ family hydrolyze ATP, converting Hsp70 to its high affinity form. The second class of co-factors (GrpE in bacteria; BAG-1 and HspBP1 in eukaryotes) serves as nucleotide exchange factors that stimulate ADP dissociation and ATP rebinding, serving to promote substrate release and chaperone recycling.

Overexpression of Hsp70 and its co-chaperones has been shown to decrease pathology associated with in vivo and in vitro models of misfolded disease-associated proteins (Auluck et al., 2002). In vivo, overexpression of Hsp70 in a Drosophila model of Parkinson's disease reduced neuron loss, although there was no effect on inclusion body formation (Klucken et al., 2004). Similarly, increased expression of Hsp70 in transgenic mice overexpressing α-synuclein reduces inclusion formation (Chen et al., 2002; Cummings et al., 2001; Warrick et al., 1999). In both Drosophila and mouse polyglutamate expansion disease models, transgenic overexpression of Hsp70 significantly improves the behavioral and neurodegenerative phenotypes (Banal et al., 2004; Muchowski and Wacker, 2005).

Given the protective effects of overexpression of molecular chaperones in neurodegenerative models, the loss of chaperone function is likely to enhance or cause neuron loss (Soti and Csermely, 2000). Chaperone function has been hypothesized to decrease upon aging, providing one explanation for the high incidence of sporadic cases of neurodegenerative disorders (Muchowski and Wacker, 2005). Similarly association of chaperones with inclusion bodies has been suggested to result in their sequestration and a concomitant loss of function (Hansen et al., 2002). In summary, loss of chaperone function can lead to progressive neuron loss.

### The Ubiquitin/Proteasome System in Neurodegenerative Disorders.

If misfolded proteins are not refolded by chaperones, a second line of cellular defense involves the degradation of abnormal proteins by the proteasomes or lysosomes. Interestingly, both of these degradation systems may functionally decline upon aging, as neurodegenerative disorders increase in incidence (Gandhi and Wood, 2005; Leroy et al., 1998; Mata et al., 2004; Ross and Poirier, 2004). Direct links of the ubiquitin-proteasome system to neurodegeneration, in particular PD, have been found with the identification of mutations in genes encoding components of this system. Mutations in parkin, an E3 ubiquitin ligase, and UCHL-L1, a ubiquitin C-terminal hydrolase L1 which acts in recycling of polyubiquitin chains back to monomeric ubiquitin, have be associated with autosomal recessive and dominant parkinsonism, respectively (Chung et al., 2001). In addition, functional deficits in proteosomal function were observed in substantia nigra extracts from postmortem brain tissue from patients with sporadic PD (McNaught et al., 2004). In agreement, unilateral infusion of lactacystin, a proteasome inhibitor, into the rat substantia nigra resulted in movement abnormalities and dopaminergic neuron loss associated with alpha-synuclein-containing inclusions (Glickman and Ciechanover, 2002).

Destruction of proteins via the ubiquitin-proteosome pathway involves first tagging the substrate by covalent attachment of ubiquitin, and then degradation of the ‘tagged’ proteins (and recycling of ubiquitin) by the proteosome (Glickman, 2000). Ubiquitination occurs via the concerted action of ubiquitin-activating enzyme, E1, and pairs of E2 ubiquitin conjugating enzymes and E3 ubiquitin ligases that catalyze the addition of ubiquitin chains to specific target proteins prior to their destruction by the proteasome. Degradation of polyubiquitinated proteins occurs by the eukaryotic 26S proteasome, comprised of the 20S proteolytic core particle (CP) and the 19S regulatory particle (RP) ‘cap’ that recognizes the ubiquitinated protein in an unknown manner. The 20S core unit is a barrel structure comprised of 2 rings of proteolytically active β subunits flanked by rings of a subunits, which control the passage of substrates and degraded products in and out of the proteasome.

The 19S regulatory particle has a base of six ATPases adjacent to the surface of the core particle and acts to select and activate substrates for proteolysis, and translocating them into the core particle. ATP hydrolysis by the regulatory particle likely regulates the affinity for protein substrate and causes conformational changes that may gate the channel of the core particle, and/or unfold the substrate and translocating it into the core particle (Deveraux et al., 1994). Three non-ATPase subunits (Rpn1, 2, and 10) with unknown function are also associated with the base. The RP lid, comprised of 8 non-ATPase subunits, is also necessary for proteolysis of ubiquitinated protein, but the exact function of this proteasome component is unclear. The localization of the RP lid at the outermost surface of the proteasome suggests it may interact with cytoplasmic proteins to further regulate proteosome function or subcellular localization.

### Proteasomal Recognition of Ubiquitinated Proteins.

One subunit of the regulatory channel, S5a, has been shown to bind ubiquitinated targets (Van Nocker et al., 1996). However, deletion of this gene in yeast (rpn10) causes only minor increases in ubiquitin-protein conjugates, suggesting alternative pathways for recognition of ubiquitinated proteins by the proteasome (Elsasser and Finley, 2005). More recently it has been shown that delivery of ubiquitinated substrates to the proteosome can be performed by ubiquitin receptors only transiently associated with the proteasome. Many of these proteins have a ubiquitin-like (UBL) domain at the amino-terminus that is directly recognized by the proteasome. They also have one or more ubiquitin-associated (UBA) domains at the C-terminus which binds ubiquitin (Glickman and Ciechanover, 2002; Glickman et al., 1998).

Although the overall structure is conserved throughout eukaryotes, many other proteins are transiently associated with the proteasome. These accessory proteins may be necessary for, or influence, substrate presentation to the proteasome and/or subcellular localization of proteasome (Leggett et al., 2002). These proteins include the ubiquitin-like protein, Rad23, ubiquitinating and deubiquitinating enzymes, and adaptor and cell cycle proteins (Dawson et al., 2006; Dawson and Dawson, 2003; Hori et al., 1998; Mayer and Fujita, 2006; Verma et al., 2004).

Of particular interest because of its structural homology to ANKRD16, which is a modulator of the sti gene (also known as Aars), is gankyrin, a protein comprised largely of six 32-amino acid ankyrin repeats (Dawson et al., 2002; Hori et al., 1998; Lam et al., 2002). Human gankyrin binds to the S6 ATPase protein, the component of regulatory particle that directly interacts with the polyubiquitin chain of proteins destined to be degraded, although the functional significance of this binding, like that of many other proteasome-interacting proteins, is not understood. Interestingly, mammalian gankyrin also forms complexes with the S6 ATPase and CDK4 kinase, p53 and the retinoblastoma (RB) protein and overexpression of gankyrin can cause cellular transformation (Higashitsuji et al., 2000). Gankyrin binds to the E3 RING finger ubiquitin ligase, Mdm2, facilitating the interaction of Mdm2 and p53, which leads to increased ubiquitination and degradation of p53 (Higashitsuji et al., 2005).

### Autophagy and Neurodegenerative Disorders.

In addition to proteasome-mediated degradation, misfolded proteins in the cell can be cleared by autophagy (Reggiori, 2006; Reggiori and Klionsky, 2002). Autophagy is the major cellular mechanism for clearance of damaged organelles and degradation of long-lived proteins. Macroautophagy involves the formation or double membrane vesicles, or autophagosomes, which envelop cytoplasmic material to be digested. These autophagosomes then fuse with the lysosome where the contents of the vesicle are degraded. Less is known about microautophagy, which also involves lysosomal-mediated digestion of cytosol, but in this case, engulfment occurs directly at the lysosomal membrane. Lastly, chaperone-mediated autophagy involves the recognition of target sequences in proteins to be degraded by cytosolic proteins. These cytosolic proteins then deliver the substrate to a receptor on the lysosome where it is then translocated into the lysosome via another chaperone located in the lysosomal lumen. Genetic screens in yeast have identified many genes necessary for autophagy, many of which have clear mammalian homologs (Yuan et al., 2003).

While microautophagy plays a normal role in recycling of cellular organelles, macroautophagy and chaperone-mediated autophagy can occur as a cellular reaction to several types of intra- and extracellular stress conditions. Although autophagosomes have been observed in degenerating neurons (Levine and Yuan, 2005), recent experimental data has suggested a protective role for autophagy in neurodegenerative diseases (Cuervo, 2004). For example, expression of disease-associated proteins, including mutant forms of alpha-synuclein, in various cell lines induces autophagy, but degradation of mutant forms associated with familial Parkinson's disease was much less efficient (Cuervo, 2004; Ravikumar et al., 2002; Ravikumar et al., 2004). Conversely, inhibition of autophagy increases accumulation of intracellular aggregates of these proteins (Ravikumar et al., 2005). Impairment of autophagy by inhibition of the microtubular motor dynein is correlated with neurodegeneration in mice and humans (Berger et al., 2006). Lastly, rapamycin, an autophagy inducer, enhances the clearance of proteins with expanded polyglutamines and mutant forms of tau (Hall and Yao, 2005; Robertson et al., 2002; Watase and Zoghbi, 2003; Zoghbi and Botas, 2002).

### Protein Misfolding-Mediated Neurodegeneration and Loss-of-Function Mutations

Using mouse and other model organisms it has been shown that overexpression of disease-associated proteins (e.g., mutant APP, Tau, SOD1, or expanded polyglutamine proteins) lead to fibrillar inclusions in the CNS (Lee et al., 2006). Misfolded proteins can mediate neuron loss in the absence of inclusion body formation.

The spontaneous sticky (sti) mutation causes ataxia concomitant with Purkinje cell degeneration in mice homozygous for this mutation. Histological analysis reveals a loss of cerebellar Purkinje cells in sti/sti mice (also known as B6.stock-sti/sti or B6.Cg-Aarssti/J) beginning at three or four weeks of age. By six weeks of age, extensive Purkinje cell loss is observed, particularly in the rostral cerebellum. This degeneration continues to progress so that most Purkinje cells have degenerated by three months. However, Purkinje cells in the caudally located lobule are resistant to this mutation, with most cells still present in 18-month old mice. Identification of the mutation in the sticky mutant mouse demonstrates that a relatively subtle loss of translational fidelity can result in misfolded protein accumulation and selective neuronal degeneration. In this case a amino acid substitution in the editing domain of AlaRS leading to the random, low frequency misincorporation of serine at alanine codons during translation (Lee et al., 2006; Oberdick et al., 1990; Smeyne et al., 1995).

### ANKRD16

ANKRD16 has been identified as a novel regulator of protein degradation pathways capable to remove misfolded proteins. ANKRD16 is a 361 amino acid protein comprised of 9 ankyrin repeats and a putative C-terminal ubiquitin-binding (UBA) domain. ANKRD 16 is ubiquitously expressed and expression is apparent in early stages of development of mice.

ANKRD 16 acts cell autonomously. ANKRD 16 is localized in the nucleus and cytoplasm. ANKRD16 relocalizes to the aggresome, the site of misfolded protein accumulation. ANKRD16 also associates with ubiquitinated proteins that spontaneously misfold to form aggresomes.

ANKRD16 suppresses the accumulation of misfolded proteins. This has been shown for sti/sti Purkinje cells and mutant fibroblasts, where the accumulation of misfolded proteins is associated death of these cells.

ANKRD16 is expressed in several isoforms with the full-length form being most efficient in the disposal of misfolded proteins.

Gene dosage and ANKRD16 expression correlate. Higher levels of ANKRD16 expression are beneficial.

### II. Definitions

A “sticky mutant cell” refers to a cell that has one or two copies of the sticky (sti) mutation. The gene underlying the sticky mutation is also known as Aars.

A “subject” refers to a vertebrate, such as for example, a mammal, or a human. Though the compositions of the present application are primarily concerned with the treatment of human subjects, they may also be employed for the treatment and diagnosis of other mammalian subjects such as dogs and cats, or other mammals, for veterinary purposes.

The term “derived from” means “obtained from” or “produced by” or “descending from”.

The term “genetically altered antibodies” means antibodies wherein the amino acid sequence has been varied from that of a native antibody. Because of the relevance of recombinant DNA techniques to this application, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with membranes and other effector functions. Changes in the variable region will be made in order to improve the antigen binding characteristics.

The term “an antigen-binding fragment of an antibody” refers to any portion of an antibody that retains the binding utility to the antigen. An exemplary antigen-binding fragment of an antibody is the heavy chain and/or light chain CDR, or the heavy and/or light chain variable region.

The term “homologous,” in the context of two nucleic acids or polypeptides refers to two or more sequences or subsequences that have at least about 85%, at least 90%, at least 95%, or higher nucleotide or amino acid residue identity, when compared and aligned for maximum correspondence, as measured using the following sequence comparison method and/or by visual inspection. In certain embodiments, the “homolog” exists over a region of the sequences that is about 50 residues in length, at least about 100 residues, at least about 150 residues, or over the full length of the two sequences to be compared.

Methods of determining percent identity are known in the art. “Percent (%) sequence identity” with respect to a specified subject sequence, or a specified portion thereof, may be defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the program WU-BLAST-2.0a19 (Altschul et al., J. Mol. Biol. 215:403-410 (1997); http://blast.wust1.edu/blast/README.htm-1) with search parameters set to default values. The HSP S and HSP S2 parameters are dynamic values and are established by the program itself depending upon the composition of the particular sequence and composition of the particular database against which the sequence of interest is being searched. A “% identity value” is determined by the number of matching identical nucleotides or amino acids divided by the sequence length for which the percent identity is being reported.

The term “specifically binds” is meant an antibody that recognizes and binds an antigen or antigenic domain such as a antigenic sequence in ANKRD16 but that does not substantially recognize and bind other antigen molecules in a sample.

The term “isolated” is meant a nucleic acid, polypeptide, or other molecule that has been separated from the components that naturally accompany it. Typically, the polypeptide is substantially pure when it is at least 60%, 70%, 80%, 90%, 95%, or even 99%, by weight, free from the proteins and naturally-occurring organic molecules with which is it naturally associated. For example, a substantially pure polypeptide may be obtained by extraction from a natural source, by expression of a recombinant nucleic acid in a cell that does not normally express that protein, or by chemical synthesis.

The term “control level” refers to the level of ANKRD 16 in a biological sample obtained from a “normal” or “healthy” individual(s) that is believed not to have a neurodegenerative disease or proteopathy. Controls may be selected using methods that are well known in the art. Once a level has become well established for a control population, array results from test biological samples can be directly compared with the known levels.

The term “test sample” refers to a biological sample obtained from a patient being tested for a neurodegenerative disease or proteopathy. As ANKRD 16 is ubiquitously expressed, the biological sample can be obtained from any part or tissue of the subject. For example the biological sample can be a tissue sample, a blood sample, a cerebrospinal fluid sample, a saliva sample, or a serum sample.

For purposes of comparison, the test sample and control biological sample are of the same type, that is, obtained from the same biological source and measure the same ANKRD16 type. The control sample can also be a standard sample that contains the same concentration of ANKRD16 that is normally found in a biological sample of the same type and that is obtained from a healthy individual. For example, there can be a standard control sample for the amounts of ANKRD16 normally found in biological samples such as blood, saliva, cerebral spinal fluid, or tissue.

The present invention also contemplates the assessment of the level of ANKRD16 present in multiple test samples obtained from the same subject, where a progressive decrease in the amount of ANKRD16 over time indicates an increased risk of a neurodegenerative disease or proteopathy and a poor prognosis.

As used herein, “the presence of lower amounts of ANKRD 16 in the test sample as compared to the control level” refers to an amount of ANKRD 16 that is significantly decreased in the test sample as compared to the amount of ANKRD16 present in a control sample. “Significantly decreased” means that the differences between the compared levels are statistically significant. The levels of ANKRD 16 can be represented by arbitrary units, for example as units obtained from a densitometer, luminometer, a Fluorescence Activated Cell Sorting (FACS) machine or an ELISA (Enzyme-Linked ImmunoSorbent Assay) plate reader.

The term “statistically significant” or “significantly” refers to statistical significance and generally means a two standard deviation (2SD) below normal, or lower, concentration of the marker.

As used herein, “higher amounts” or “higher levels” refers to statistical significance and generally means a two standard deviation (2SD) above the amount or level to be compared.

As used herein, “ANKRD16” or “Ankrd16” refers to ANKRD16 protein or nucleic acid (DNA or RNA). Full length ANKRD16 protein is 361 amino acids and is comprised of 9 ankyrin repeats and a putative C-terminal ubiquitin-binding (UBA) domain. The full length human ANKRD16 nucleic acid and protein sequences (human ANKRD16A) are shown in SEQ ID NO: 1 and SEQ ID NO: 2, respectively (Mouse ANKRD16D, SEQ ID NO.'s: 21 and 22, respectively). ANKRD16 protein is also expressed in shorter isoforms, for example, as depicted in SEQ ID NO.'s: 3 and 4 (Human isoform 16B); SEQ ID NO.'s: 4 and 5 (Human isoform 16C); SEQ ID NO.'s: 15 and 16 (Mouse isoform 16A); SEQ ID NO.'s: 17 and 18 (Mouse isoform 16B); SEQ ID NO.'s: 19 and 20 (Mouse isoform 16C). The term ANKRD 16 also encompasses species variants, homologues, allelic forms, mutant forms, and equivalents thereof. Protein GenBank accession numbers for ANKRD16 include, but are not limited to CAK05078; CAM20367; CAI41072 ; NP_{—}796242; Q6P6B7; Q499M5; NP_{—}001017563; AAI20322; AAH92927; NP_{—}001009943; NP_{—}001009942; NP_{—}001009941; NP_{—}061919; EDL78592; EDL78591; EDL78590; NP_{—}001068799; EDL08036; EDL08035; EDL08034; EDL08033; NP_{—}001028870; EAW86429; XP_{—}414984; AAI16231; AAI16230; XP_{—}001145211; XP_{—}001145289; XP_{—}001145131; XP_{—}507639; AAH99837; AAH62346, where corresponding nucleic acid sequences can be found.

### III. Activators of ANKRD16

In certain embodiments, the activators of ANKRD16 include any molecules that directly or indirectly increase, agonize or activate ANKRD16 biological activities.

In another aspect, the activators of the present application activate at least one, or all, biological activities of ANKRD16. The biological activities of ANKRD16 include suppressing the accumulation of misfolded proteins.

In certain embodiments, the activators of the present application may have at least one activity selected from the group consisting of: 1) treating neurodegenerative disease; 2) treating a disease caused by misfolded protein; or 3) acting as a diagnostic or prognostic marker.

In one embodiment, the activators treat neurodegenerative disease or a disease caused by misfolded protein in vivo (such as in a subject), such as for example, by at least 10%, 25%, 50%, 75%, or 90%.

In another embodiment, the activators inhibit disease symptoms of a neurodegenerative disease or a disease caused by misfolded protein, such as for example, by at least 10%, 25%, 50%, 75%, or 90%.

In one aspect, the activators directly interact with ANKRD16. In certain embodiments, the activators are proteins or peptides. In certain embodiments, the proteins bind to ANKRD16. In certain embodiments, the activators are antibodies or antibody fragments that bind to ANKRD16 and promote at least one biological activity of ANKRD 16. In certain embodiments, the activators are non-immunoglobulin binding proteins that bind to ANKRD16 and promote at least one biological activity of ANKRD16.

Alternatively, the activators interact with and regulate the upstream or downstream components of the ANKRD16 signaling pathway and indirectly increase the activities of ANKRD 16. Accordingly, any molecules capable of regulating this pathway can be candidate activators. Yeast two-hybrid and variant screens offer methods for identifying endogenous additional interacting proteins of the components of the ANKRD16 signaling pathways (Finley et al. in DNA Cloning-Expression Systems: A Practical Approach, eds. Glover et al. (Oxford University Press, Oxford, England), pp. 169-203 (1996); Fashema et al., Gene 250: 1-14 (2000); Drees, CUK Opin Chem Biol 3: 64-70 (1999); Vidal et al. Nucleic Acids Res. 27:9191-29 (1999); and U.S. Pat. No. 5,928,868). Mass spectrometry is an alternative method for the elucidation of protein complexes (reviewed in, e. g., Pandley et al., Nature 405: 837-846 (2000); Yates, 3rd, Trends Genet 16: 5-8 (2000)).

In yet another aspect, the activators may activate the protein expression of ANKRD16. ANKRD16 expression can be regulated at the level of transcription, such as, by a regulator of transcription factors of ANKRD16, or at the level of mRNA splicing, translation or post-translation.

The activators can also be nucleic acids, including, but not limited to, aptamers or microRNAs that bind to ANKRD16. The DNA sequence of ANKRD16 is known in the art and disclosed herein.

The activators of the present application also include small molecules, which may activate the activity of proteins with enzymatic function, and/or the interactions of said proteins. Chemical agents, referred to in the art as “small molecule” compounds are typically organic, non-peptide molecules, having a molecular weight less than 10,000, less than 5,000, less than 1,000, or less than 500 daltons. This class of activators includes chemically synthesized molecules, for instance, compounds from combinatorial chemical libraries. Synthetic compounds may be rationally designed or identified based on known or inferred properties of the ANKRD16 protein or may be identified by screening compound libraries. Alternative appropriate activators of this class are natural products, particularly secondary metabolites from organisms such as plants or fungi, which can also be identified by screening compound libraries for ANKRD16-modulating activity. Methods for generating and obtaining compounds are well known in the art (Schreiber S L, Science 151: 1964-1969(2000); Radmann J. and Gunther J., Science 151: 1947-1948 (2000)).

Peptidomimetics can be compounds in which at least a portion of a subject polypeptide of the disclosure (such as for example, a polypeptide comprising an amino acid sequence of greater than 90% sequence identity to the amino acid sequence of a soluble portion of a naturally occurring ANKRD16 protein) is modified, and the three dimensional structure of the peptidomimetic remains substantially the same as that of the subject polypeptide. Peptidomimetics may be analogues of a subject polypeptide of the disclosure that are, themselves, polypeptides containing one or more substitutions or other modifications within the subject polypeptide sequence. Alternatively, at least a portion of the subject polypeptide sequence may be replaced with a nonpeptide structure, such that the three-dimensional structure of the subject polypeptide is substantially retained. In other words, one, two or three amino acid residues within the subject polypeptide sequence may be replaced by a non-peptide structure. In addition, other peptide portions of the subject polypeptide may, but need not, be replaced with a non-peptide structure. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of disorders in a human or animal. It should be noted that peptidomimetics may or may not have similar two-dimensional chemical structures, but share common three-dimensional structural features and geometry. Each peptidomimetic may further have one or more unique additional binding elements.

The present invention also contemplates prodrugs as ANKRD16 activators. A prodrug is a pharmacological substance which is administered in an inactive (or significantly less active) form. Once administered, the prodrug is metabolized in vivo into an active compound.

In another aspect, the ANKRD16 activators are molecules that inhibit the expression or activity of an ANKRD16 inhibitor, such as RNAi constructs (including shRNA-based or microRNA-based siRNA) that target endogenous antisense polynucleotides of ANKRD16. RNAi-based technology is known in the art and has been widely used to silence or inhibit the expression of target polynucleotides.

### IV. Polypeptides

In certain aspects, the invention relates to a polypeptide comprising an isoform of ANKRD16 or a fragment thereof. In certain embodiments, the polypeptide is selected from SEQ ID Nos: 2, 4, 6, 8, 10, 12, 14 or 22. In certain embodiments, the subject polypeptide is a monomer and is functional.

In certain embodiments, a functional variant of an ANKRD16 polypeptide comprises an amino acid sequence that is at least 90%, 95%, 97%, 99% or 100% identical to the amino acid sequence defined in SEQ ID Nos: 2, 4, 6, 8, 10, 12, 14 or 22.

In certain embodiments, the present invention contemplates making functional variants by modifying the structure of the subject polypeptide for such purposes as enhancing therapeutic or prophylactic efficacy, or stability (e.g., ex vivo shelf life and resistance to proteolytic degradation in vivo). Such modified polypeptides are considered functional equivalents of the naturally occurring ANKRD16 polypeptide. Modified polypeptides can be produced, for instance, by amino acid substitution, deletion, or addition or by glycosylation. For instance, it is reasonable to expect, for example, that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains.

This invention further contemplates a method of generating sets of combinatorial mutants of the ANKRD16 polypeptides, as well as truncation mutants, and is especially useful for identifying functional variant sequences. The purpose of screening such combinatorial libraries may be to generate, for example, polypeptide variants which can act as activators of ANKRD16. Combinatorially-derived variants can be generated which have a selective potency relative to a naturally occurring polypeptide. Such variant proteins, when expressed from recombinant DNA constructs, can be used in gene therapy protocols. Likewise, mutagenesis can give rise to variants which have intracellular half-lives dramatically different than the corresponding wild-type polypeptide. For example, the altered protein can be rendered either more stable or less stable to proteolytic degradation or other cellular process which result in destruction of, or otherwise inactivation of the protein of interest (e.g., a polypeptide). Such variants, and the genes which encode them, can be utilized to alter the subject polypeptide levels by modulating their half-life. For instance, a short half-life can give rise to more transient biological effects and, when part of an inducible expression system, can allow tighter control of recombinant polypeptide levels within the cell. As above, such proteins, and particularly their recombinant nucleic acid constructs, can be used in gene therapy protocols. Half-life can also be increased by adding moieties, such as polyethylene glycol, transferrin, albumin or the Fc fragment.

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential polypeptide sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos: 5,223,409, 5,198,346, and 5,096,815).

Alternatively, other forms of mutagenesis can be utilized to generate a combinatorial library. For example, polypeptide variants (e.g., constitutively active forms) can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of the subject polypeptide.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of the subject polypeptides. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In certain embodiments, the subject polypeptides of the invention include a small molecule such as a peptide and a peptidomimetic. As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of the ANKRD16 polypeptides.

To illustrate, by employing scanning mutagenesis to map the amino acid residues of a polypeptide which are involved in binding to another protein, peptidomimetic compounds can be generated which mimic those residues involved in binding. For instance, non-hydrolyzable peptide analogs of such residues can be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., (1985) Tetrahedron Lett 26:647; and Sato et al., (1986) J Chem Soc Perkin Trans 1:1231), and b-aminoalcohols (Gordon et al., (1985) Biochem Biophys Res Commun 126:419; and Dann et al., (1986) Biochem Biophys Res Commun 134:71).

In certain embodiments, the polypeptides of the invention may further comprise post-translational modifications. Such modifications include, but are not limited to, acetylation, carboxylation, glycosylation, phosphorylation, lipidation, and acylation. As a result, the modified polypeptides may contain non-amino acid elements, such as polyethylene glycols, lipids, poly- or mono-saccharide, and phosphates. Effects of such non-amino acid elements on the functionality of a polypeptide may be tested for its activating role in ANKRD16 function.

In certain aspects, functional variants or modified forms of the subject polypeptides include fusion proteins having at least a portion of the polypeptide and one or more fusion domains. Well known examples of such fusion domains include, but are not limited to, polyhistidine, Glu-Glu, glutathione S transferase (GST), thioredoxin, protein A, protein G, and an immunoglobulin heavy chain constant region (Fc), maltose binding protein (MBP), which are particularly useful for isolation of the fusion proteins by affinity chromatography. For the purpose of affinity purification, relevant matrices for affinity chromatography, such as glutathione-, amylase-, and nickel- or cobalt-conjugated resins are used. Another fusion domain well known in the art is green fluorescent protein (GFP). Fusion domains also include “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins there from. The liberated proteins can then be isolated from the fusion domain by subsequent chromatographic separation. In certain embodiments, the polypeptides of the present invention contain one or more modifications that are capable of stabilizing the polypeptides. For example, such modifications enhance the in vitro half-life of the polypeptides, enhance circulatory half life of the polypeptides or reducing proteolytic degradation of the polypeptides.

In certain embodiments, the polypeptides of the invention may further comprise a signal sequence for cell penetration, such as CPP peptides (also called membrane-translocating sequences, or protein transduction domains) or activated CPP peptides. Techniques for expressing CPP peptide-fusion proteins are known in the art (see, e.g., Jones et al. British Journal of Pharmacology (2005) 145, 1093-1102, WO2006125134, Jiang et al., Proc. Natl. Acad. Sci. USA, (2004), 101, 17867-17872). Briefly, certain polycationic sequences such as CPPs can bring covalently attached payloads into mammalian cells without requiring specific receptors. A variety of multicationic oligomers, including guanidinium-rich sequences, or 6-12 consecutive arginines are known to be highly effective. D-amino acids are at least as good as natural L-amino acids and possibly better because the unnatural isomers resist proteolysis. By modifying the CPP a target delivery is possible, e.g. by introducing linker sequences which can be cleaved for example by tissue-specific enzymes.

In certain embodiments, polypeptides (unmodified or modified) of the invention can be produced by a variety of art-known techniques. For example, such polypeptides can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the polypeptides, fragments or variants thereof may be recombinantly produced using various expression systems as is well known in the art (also see below).

### V. Nucleic Acids Encoding Polypeptides

In certain aspects, the invention relates to isolated and/or recombinant nucleic acids encoding an ANKRD16 polypeptide. The subject nucleic acids may be single-stranded or double-stranded, DNA or RNA molecules. These nucleic acids are useful as therapeutic agents. For example, these nucleic acids are useful in making recombinant polypeptides which are administered to a cell or an individual as therapeutics. Alternative, these nucleic acids can be directly administered to a cell or an individual as therapeutics such as in gene therapy.

In certain embodiments, the invention provides isolated or recombinant nucleic acid sequences that are at least 80%, 85%, 90%, 95%, 97%, 98%, 99% or 100% identical to a region of the nucleotide sequences of SEQ ID Nos: 1, 3, 5, 7, 9, 11, or 13. One of ordinary skill in the art will appreciate that nucleic acid sequences complementary to the subject nucleic acids, and variants of the subject nucleic acids are also within the scope of this invention. In further embodiments, the nucleic acid sequences of the invention can be isolated, recombinant, and/or fused with a heterologous nucleotide sequence, or in a DNA library.

In other embodiments, nucleic acids of the invention also include nucleotide sequences that hybridize under highly stringent conditions to the nucleotide sequences of SEQ ID Nos: 1, 3, 5, 7, 9, 11, or 13 or complement sequences thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature.

Isolated nucleic acids which differ from the subject nucleic acids due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this invention.

In certain embodiments, the recombinant nucleic acids of the invention may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In certain aspects of the invention, the subject nucleic acid is provided in an expression vector comprising a nucleotide sequence encoding an ANKRD16 polypeptide and operably linked to at least one regulatory sequence. Regulatory sequences are art-recognized and are selected to direct expression of the polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a polypeptide. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

This invention also pertains to a host cell transfected with a recombinant gene including a coding sequence for one or more of the subject polypeptide. The host cell may be any prokaryotic or eukaryotic cell. For example, a polypeptide of the invention may be expressed in bacterial cells such as E. coli, Bacillus, insect cells (e.g., using a baculovirus expression system), yeast, algae, plant or mammalian cells. Other suitable host cells are known to those skilled in the art. The nucleic acid sequences used for the ANKRD16 expression may be adapted for optimized codon usage of the organism used for the expression as it is known in the art.

Accordingly, the present invention further pertains to methods of producing the subject polypeptides. For example, a host cell transfected with an expression vector encoding an ANKRD16 polypeptide can be cultured under appropriate conditions to allow expression of the ANKRD16 polypeptide to occur. The ANKRD16 polypeptide may be secreted and isolated from a mixture of cells and medium containing the polypeptides. Alternatively, the polypeptides may be retained cytoplasmically or in a membrane fraction and the cells harvested, lysed and the protein isolated. A cell culture includes host cells, media and other byproducts. Suitable media for cell culture are well known in the art. The polypeptides can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptides. In a preferred embodiment, the polypeptide is a fusion protein containing a domain which facilitates its purification.

A recombinant nucleic acid of the invention can be produced by ligating the cloned gene, or a portion thereof, into a vector suitable for expression in either prokaryotic cells, eukaryotic cells (plant, yeast, avian, insect or mammalian), or both.

Recombinant protein expression also can be achieved in a transgenic animal, e.g. rodents, ovine, porcine, bovidea (bovine, goat, sheep), rabbit or avian. Expression vehicles for production of a recombinant polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant SLC5A8 polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

### VI. Antibodies

In one embodiment, the application discloses antibodies against ANKRD16. These antibodies may be in a polyclonal or monoclonal form and may be immunoreactive with at least one epitope of ANKRD16, such as for example, a human ANKRD16 and/or mouse ANKRD16. In certain embodiments, the antibodies may bind to a) a full-length ANKRD16 polypeptide, or b) a functionally active fragment or derivative thereof. In certain embodiments, the antibodies may bind to a specific isoform of ANKRD16. In certain embodiments, the antibodies bind to any of SEQ ID Nos: 2, 4, 6, 8, 10, 12, 14 or 22.

In some embodiments, the anti-ANKRD16 antibody binds to a ANKRD16 polypeptide with a K_{D} of 1×10⁻⁶ M or less. In still other embodiments, the antibody binds to a ANKRD16 polypeptide with a K_{D} of 1×10⁻⁷ M, 3×10⁻⁸M, 2×10⁹M, 1×10⁻¹⁰ M, 1×10⁻¹¹ M, or 5×10⁻¹² M or less.

The anti-ANKRD16 antibodies of the present application include antibodies having all types of constant regions, including IgM, IgG, IgD, IgA and IgE, and any isotype, including IgG1, IgG2a, IgG2b, IgG3 and IgG4. The light chains of the antibodies can either be kappa light chains or lambda light chains.

In another aspect, the antibodies of the present application activate at least one, or all, biological activities of ANKRD16. The biological activities of ANKRD16 include suppressing the accumulation of misfolded proteins.

In certain embodiments, the antibodies of the present application may have at least one activity selected from the group consisting of: 1) treating neurodegenerative disease; 2) treating a disease caused by misfolded protein; or 3) acting as a diagnostic or prognostic marker.

In one embodiment, the antibodies inhibit neurodegenerative disease or a disease caused by misfolded protein in vivo (such as in a subject), such as for example, by at least 10%, 25%, 50%, 75%, or 90%.

In another embodiment, the antibodies inhibit disease symptoms of a neurodegenerative disease or a disease caused by misfolded protein, such as for example, by at least 10%, 25%, 50%, 75%, or 90%.

The present application provides for the polynucleotide molecules encoding the antibodies and antibody fragments and their analogs described herein. Because of the degeneracy of the genetic code, a variety of nucleic acid sequences encode each antibody amino acid sequence. The desired nucleic acid sequences can be produced by de novo solid-phase DNA synthesis or by PCR mutagenesis of an earlier prepared variant of the desired polynucleotide. In one embodiment, the codons that are used comprise those that are typical for human or mouse (see, e.g., Nakamura, Y., Nucleic Acids Res. 28: 292 (2000)).

The present application includes the monoclonal antibodies that bind to substantially the same epitope as any one of the exemplified antibodies. Two antibodies are said to bind to substantially the same epitope of a protein if amino acid mutations in the protein that reduce or eliminate binding of one antibody also reduce or eliminate binding of the other antibody, and/or if the antibodies compete for binding to the protein, i.e., binding of one antibody to the protein reduces or eliminates binding of the other antibody. The determination of whether two antibodies bind substantially to the same epitope is accomplished by the methods known in the art, such as a competition assay. In conducting an antibody competition study between a control antibody (for example, one of the anti-ANKRD16 antibodies described herein) and any test antibody, one may first label the control antibody with a detectable label, such as, biotin, enzymatic, radioactive label, or fluorescence label to enable the subsequent identification. An antibody that binds to substantially the same epitope as the control antibody should be able to compete for binding and thus should reduce control antibody binding, as evidenced by a reduction in bound label.

The polyclonal forms of the anti-ANKRD16 antibodies are also included in the present application. In certain embodiments, these antibodies activate at least one activity of ANKRD16, or bind to the ANKRD16 epitopes as the described monoclonal antibodies in the present application. Polyclonal antibodies can be produced by the method described herein.

Antibodies against ANKRD16 of all species of origins are included in the present application. Non-limiting exemplary natural antibodies include antibodies derived from human, chicken, goats, sheep, horse, llama, camel, rabbits and rodents (e.g., rats, mice and hamsters), including transgenic animals (rodents, rabbits, goats) genetically engineered to produce human antibodies (see, e.g., Lonberg et al., WO93/12227; U.S. Pat. No. 5,545,806; and Kucherlapati, et al., WO91/10741; U.S. Pat. No. 6,150,584, which are herein incorporated by reference in their entirety). Natural antibodies are the antibodies produced by a host animal. In one embodiment, the antibody is an isolated monoclonal antibody that binds to and/or activates ANKRD16.

Recombinant antibodies against ANKRD16 are also included in the present application. These recombinant antibodies have the same amino acid sequence as the natural antibodies or have altered amino acid sequences of the natural antibodies in the present application. They can be made in any expression systems including both prokaryotic and eukaryotic expression systems or using phage display methods (see, e.g., Dower et al., WO91/17271 and McCafferty et al., WO92/01047; U.S. Pat. No. 5,969,108, which are herein incorporated by reference in their entirety).

Antibodies can be engineered in numerous ways. They can be made as single-chain antibodies (including small modular immunopharmaceuticals or SMIPs™), Fab and F(ab′)₂ fragments, etc. Antibodies can be humanized, chimerized, deimmunized, or fully human. Numerous publications set forth the many types of antibodies and the methods of engineering such antibodies. For example, see U.S. Pat. Nos. 6,355,245; 6,180,370; 5,693,762; 6,407,213; 6,548,640; 5,565,332; 5,225,539; 6,103,889; and 5,260,203.

Antibodies with engineered or variant constant or Fc regions can be useful in modulating effector functions, such as, for example, antigen-dependent cytotoxicity (ADCC) and complement-dependent cytotoxicity (CDC). Such antibodies with engineered or variant constant or Fc regions may be useful in instances where ANKRD16 is expressed in normal tissue, for example; variant anti-ANKRD16 antibodies without effector function in these instances may elicit the desired therapeutic response while not damaging normal tissue.

Accordingly, certain aspects and methods of the present disclosure relate to anti-ANKRD16 antibodies with altered effector functions that comprise one or more amino acid substitutions, insertions, and/or deletions. In certain embodiments, such a variant anti-ANKRD16 antibody exhibits reduced or no effector function. In particular embodiments, a variant antibody comprises a G2/G4 construct in place of the G1 domain (see Mueller et al. Mol Immunol. 1997 April; 34(6):441-52).

In addition to swapping the G1 domain with a G2/G4 construct as presented herein, anti-ANKRD16 antibodies with reduced effector function may be produced by introducing other types of changes in the amino acid sequence of certain regions of the antibody. Such amino acid sequence changes include but are not limited to the Ala-Ala mutation described by Bluestone et al. (see WO 94/28027 and WO 98/47531; also see Xu et al. 2000 Cell Immunol 200; 16-26). Thus in certain embodiments, anti-ANKRD16 antibodies with mutations within the constant region including the Ala-Ala mutation may be used to reduce or abolish effector function. According to these embodiments, the constant region of an anti-ANKRD16 antibody comprises a mutation to an alanine at position 234 or a mutation to an alanine at position 235. Additionally, the constant region may contain a double mutation: a mutation to an alanine at position 234 and a second mutation to an alanine at position 235. In one embodiment, the anti-ANKRD16 antibody comprises an IgG4 framework, wherein the Ala-Ala mutation would describe a mutation(s) from phenylalanine to alanine at position 234 and/or a mutation from leucine to alanine at position 235. In another embodiment, the anti-ANKRD16 antibody comprises an IgG 1 framework, wherein the Ala-Ala mutation would describe a mutation(s) from leucine to alanine at position 234 and/or a mutation from leucine to alanine at position 235. An anti-ANKRD16 antibody may alternatively or additionally carry other mutations, including the point mutation K322A in the CH2 domain (Hezareh et al. 2001 J Virol. 75: 12161-8).

Changes within the hinge region also affect effector functions. For example, deletion of the hinge region may reduce affinity for Fc receptors and may reduce complement activation (Klein et al. 1981 Proc Natl Acad Sci U S A. 78: 524-528). The present disclosure therefore also relates to antibodies with alterations in the hinge region.

In particular embodiments, anti-ANKRD16 antibodies may be modified to either enhance or inhibit complement dependent cytotoxicity (CDC). Modulated CDC activity may be achieved by introducing one or more amino acid substitutions, insertions, or deletions in an Fc region of the antibody (see, e.g., U.S. Pat. No. 6,194,551). Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO99/51642, Duncan & Winter Nature 322: 738-40 (1988); U.S. Pat. No. 5,648,260; U.S. Pat. No. 5,624,821; and WO94/29351. Homodimeric antibodies with enhanced activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

Another potential means of modulating effector function of antibodies includes changes in glycosylation. This topic has been recently reviewed by Raju who summarized the proposed importance of the oligosaccharides found on human IgGs with their degree of effector function (Raju, T S. BioProcess International April 2003. 44-53). According to Wright and Morrison, the microheterogeneity of human IgG oligosaccharides can affect biological functions such as CDC and ADCC, binding to various Fc receptors, and binding to Clq protein (Wright A. & Morrison S L. TIBTECH 1997, 15: 26-32). It is well documented that glycosylation patterns of antibodies can differ depending on the producing cell and the cell culture conditions (Raju, T S. BioProcess International April 2003. 44-53). Such differences can lead to changes in both effector function and pharmacokinetics (Israel et al. Immunology. 1996; 89(4):573-578; Newkirk et al. P. Clin. Exp. 1996; 106(2):259-64). Differences in effector function may be related to the IgGs ability to bind to the Fcγ receptors (FcγRs) on the effector cells. Shields, et al., have shown that IgG, with variants in amino acid sequence that have improved binding to FcγR, can exhibit up to 100% enhanced ADCC using human effector cells (Shields et al. J Biol Chem. 2001 276(9):6591-604). While these variants include changes in amino acids not found at the binding interface, both the nature of the sugar component as well as its structural pattern may also contribute to the differences observed. In addition, the presence or absence of fucose in the oligosaccharide component of an IgG can improve binding and ADCC (Shields et al. J Biol Chem. 2002; 277(30):26733-40). An IgG that lacked a fucosylated carbohydrate linked to Asn²⁹⁷ exhibited normal receptor binding to the Fcγ receptor. In contrast, binding to the FcγRIIA receptor was improved 50% and accompanied by enhanced ADCC, especially at lower antibody concentrations.

Work by Shinkawa, et al., demonstrated that an antibody to the human IL-5 receptor produced in a rat hybridoma showed more than 50% higher ADCC when compared to the antibody produced in Chinese hamster ovary cells (CHO) (Shinkawa et al. J Biol Chem. 2003 278(5):3466-73). Monosaccharide composition and oligosaccharide profiling showed that the rat hybridoma-produced IgG had a lower content of fucose than the CHO-produced protein. The authors concluded that the lack of fucosylation of an IgG1 has a critical role in enhancement of ADCC activity.

A different approach was taken by Umana, et al., who changed the glycosylation pattern of chCE7, a chimeric IgG1 anti-neuroblastoma antibody (Umana et al. Nat Biotechnol. 1999 February; 17(2): 176-80). Using tetracycline, they regulated the activity of a glycosyltransferase enzyme (GnnII) which bisects oligosaccharides that have been implicated in ADCC activity. The ADCC activity of the parent antibody was barely above background level. Measurement of ADCC activity of the chCE7 produced at different tetracycline levels showed an optimal range of GnTIH expression for maximal chCE7 in vitro ADCC activity. This activity correlated with the level of constant region-associated, bisected complex oligosaccharide. Newly optimized variants exhibited substantial ADCC activity. Similarly, Wright and Morrison produced antibodies in a CHO cell line deficient in glycosylation (1994 J Exp Med 180: 1087-1096) and showed that antibodies produced in this cell line were incapable of complement-mediated cytolysis. Thus as known alterations that affect effector function include modifications in the glycosylation pattern or a change in the number of glycosylated residues, the present disclosure relates to a ANKRD16 antibody wherein glycosylation is altered to either enhance or decrease effector function(s) including ADCC and CDC. Altered glycosylation includes a decrease or increase in the number of glycosylated residues as well as a change in the pattern or location of glycosylated residues.

Still other approaches exist for the altering effector function of antibodies. For example, antibody-producing cells can be hypermutagenic, thereby generating antibodies with randomly altered nucleotide and polypeptide residues throughout an entire antibody molecule (see WO 2005/011735). Hypermutagenic host cells include cells deficient in DNA mismatch repair. Antibodies produced in this manner may be less antigenic and/or have beneficial pharmacokinetic properties. Additionally, such antibodies may be selected for properties such as enhanced or decreased effector function(s).

It is further understood that effector function may vary according to the binding affinity of the antibody. For example, antibodies with high affinity may be more efficient in activating the complement system compared to antibodies with relatively lower affinity (Marzocchi-Machado et al. 1999 Immunol Invest 28: 89-101). Accordingly, an antibody may be altered such that the binding affinity for its antigen is reduced (e.g., by changing the variable regions of the antibody by methods such as substitution, addition, or deletion of one or more amino acid residues). An anti-ANKRD16 antibody with reduced binding affinity may exhibit reduced effector functions, including, for example, reduced ADCC and/or CDC.

In certain embodiments, ANKRD16 antibodies utilized in the present disclosure are especially indicated for diagnostic and therapeutic applications as described herein. Accordingly, ANKRD16 antibodies may be used in therapies, including combination therapies, in the diagnosis and prognosis of disease, as well as in the monitoring of disease progression.

In the therapeutic embodiments of the present disclosure, bispecific antibodies are contemplated. Bispecific antibodies may be monoclonal, human or humanized antibodies that have binding specificities for at least two different antigens. In the present case, one of the binding specificities is for the ANKRD16 antigen on a cell, the other one is for any other antigen, such as for example, a cell-surface protein or receptor or receptor subunit.

The genetically altered anti-ANKRD16 antibodies should be functionally equivalent to the above-mentioned natural antibodies. In certain embodiments, modified antibodies provide improved stability or/and therapeutic efficacy. Examples of modified antibodies include those with conservative substitutions of amino acid residues, and one or more deletions or additions of amino acids that do not significantly deleteriously alter the antigen binding utility. Substitutions can range from changing or modifying one or more amino acid residues to complete redesign of a region as long as the therapeutic utility is maintained. Antibodies of this application can be modified post-translationally (e.g., acetylation, and/or phosphorylation) or can be modified synthetically (e.g., the attachment of a labeling group). In certain embodiments, genetically altered antibodies are chimeric antibodies and humanized antibodies.

The chimeric antibody is an antibody having a variable region and a constant region derived from two different antibodies, such as for example, derived from separate species. In certain embodiments, the variable region of the chimeric antibody is derived from murine and the constant region is derived from human.

The genetically altered antibodies used in the present application include humanized antibodies that bind to and activate ANKRD16 activity. In one embodiment, said humanized antibody comprising CDRs of a mouse donor immunoglobulin and heavy chain and light chain frameworks and constant regions of a human acceptor immunoglobulin. The method of making humanized antibody is disclosed in U.S. Pat. Nos: 5,530,101; 5,585,089; 5,693,761; 5,693,762; and 6,180,370 each of which is incorporated herein by reference in its entirety.

Anti-ANKRD16 fully human antibodies are also included in the present application. In one embodiment of the present application, said fully human antibodies promote the activities of ANKRD16 described herein.

Fragments of the anti-ANKRD16 antibodies, which retain the binding specificity to ANKRD16, are also included in the present application. Examples of these antigen-binding fragments include, but are not limited to, partial or full heavy chains or light chains, variable regions, or CDR regions of any anti-ANKRD16 antibodies described herein.

In one embodiment of the application, the antibody fragments are truncated chains (truncated at the carboxyl end). In certain embodiments, these truncated chains possess one or more immunoglobulin activities (e.g., complement fixation activity). Examples of truncated chains include, but are not limited to, Fab fragments (consisting of the VL, VH, CL and CH1 domains); Fd fragments (consisting of the VH and CH1 domains); Fv fragments (consisting of VL and VH domains of a single chain of an antibody); dab fragments (consisting of a VH domain); isolated CDR regions; (Fab′)₂ fragments, bivalent fragments (comprising two Fab fragments linked by a disulphide bridge at the hinge region). The truncated chains can be produced by conventional biochemical techniques, such as enzyme cleavage, or recombinant DNA techniques, each of which is known in the art. These polypeptide fragments may be produced by proteolytic cleavage of intact antibodies by methods well known in the art, or by inserting stop codons at the desired locations in the vectors using site-directed mutagenesis, such as after CH1 to produce Fab fragments or after the hinge region to produce (Fab′)₂ fragments. Single chain antibodies may be produced by joining VL- and VH-coding regions with a DNA that encodes a peptide linker connecting the VL and VH protein fragments

This application provides fragments of anti-ANKRD16 antibodies, which may comprise a portion of an intact antibody, such as for example, the antigen-binding or variable region of the intact antibody. Examples of antibody fragments include Fab, Fab′, F(ab′)₂, and Fv fragments; diabodies; linear antibodies (Zapata et al., Protein Eng. 1995; 8(10): 1057-1062); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment of an antibody yields an F(ab′)₂ fragment that has two antigen-combining sites and is still capable of cross-linking antigen.

“Fv” usually refers to the minimum antibody fragment that contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the CDRs confer antigen-binding specificity to the antibody. However, even a single variable domain (or half of an Fv comprising three CDRs specific for an antigen) has the ability to recognize and bind antigen, although likely at a lower affinity than the entire binding site.

Thus, in certain embodiments, the antibodies of the application may comprise 1, 2, 3, 4, 5, 6, or more CDRs that recognize ANKRD16.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

“Single-chain Fv” or “scFv” antibody fragments comprise the V_{H} and V_{L} domains of an antibody, wherein these domains are present in a single polypeptide chain. In certain embodiments, the Fv polypeptide further comprises a polypeptide linker between the V_{H} and V_{L} domains that enables the scFv to form the desired structure for antigen binding. For a review of scFv see Pluckthun in The Pharmacology of Monoclonal Antibodies, vol. 113, Rosenburg and Moore, eds. (Springer-Verlag: New York, 1994), pp. 269-315.

SMIPs are a class of single-chain peptides engineered to include a target binding region and effector domain (CH2 and CH3 domains). See, e.g., U.S. Patent Application Publication No. 20050238646. The target binding region may be derived from the variable region or CDRs of an antibody, e.g., an anti-ANKRD16 antibody of the application. Alternatively, the target binding region is derived from a protein that binds ANKRD16.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90: 6444-6448 (1993).

An “isolated” antibody is one that has been identified and separated and/or recovered from a component of its natural environment. Contaminating components of its natural environment are materials that would interfere with diagnostic or therapeutic uses for the antibody, and may include enzymes, hormones, and other proteinaceous or nonproteinaceous solutes. In specific embodiments, the antibody will be purified to greater than 95% by weight of antibody as determined by the Lowry method, or greater than 99% by weight, to a degree that complies with applicable regulatory requirements for administration to human patients (e.g., substantially pyrogen-free), to a degree sufficient to obtain at least 15 residues of N-terminal or internal amino acid sequence by use of a spinning cup sequenator, or to homogeneity by SDS-PAGE under reducing or nonreducing conditions using Coomassie blue or, such as for example, silver stain. Isolated antibody includes the antibody in situ within recombinant cells, since at least one component of the antibody's natural environment will not be present. Ordinarily, however, isolated antibody will be prepared by at least one purification step, for example, an affinity chromatography step, an ion (anion or cation) exchange chromatography step, or a hydrophobic interaction chromatography step.

It is well known that the binding to a molecule (or a pathogen) of antibodies with an Fc region assists in the processing and clearance of the molecule (or pathogen). The Fc portions of antibodies are recognized by specialized receptors expressed by immune effector cells. The Fc portions of IgG1 and IgG3 antibodies are recognized by Fc receptors present on the surface of phagocytic cells such as macrophages and neutrophils, which can thereby bind and engulf the molecules or pathogens coated with antibodies of these isotypes (Janeway et al., Immunobiology 5th edition, page 147, Garland Publishing (New York, 2001)).

In certain embodiments, single chain antibodies, and chimeric, humanized or primatized (CDR-grafted) antibodies, as well as chimeric or CDR-grafted single chain antibodies, comprising portions derived from different species, are also encompassed by the present disclosure as antigen-binding fragments of an antibody. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., U.S. Pat. Nos. 4,816,567 and 6,331,415; U.S. Pat. No. 4,816,397; European Patent No. 0,120,694; WO 86/01533; European Patent No. 0,194,276 B1; U.S. Pat. No. 5,225,539; and European Patent No. 0,239,400 B1. See also, Newman et al., BioTechnology, 10: 1455-1460 (1992), regarding primatized antibody. See, e.g., Ladner et al., U.S. Pat. No. 4,946,778; and Bird et al., Science, 242: 423-426 (1988)), regarding single chain antibodies.

In addition, functional fragments of antibodies, including fragments of chimeric, humanized, primatized or single chain antibodies, can also be produced. Functional fragments of the subject antibodies retain at least one binding function and/or modulation function of the full-length antibody from which they are derived. In certain embodiments, functional fragments retain an antigen-binding function of a corresponding full-length antibody (such as for example, ability of anti-ANKRD16 antibody to bind ANKRD16).

Since the immunoglobulin-related genes contain separate functional regions, each having one or more distinct biological activities, the genes of the antibody fragments may be fused to functional regions from other genes (e.g., enzymes, U.S. Pat. No. 5,004,692, which is incorporated by reference in its entirety) to produce fusion proteins or conjugates having novel properties.

### VII. Non-Immunoglobulin Binding Proteins

In addition to antibodies, many other protein domains mediate specific high-affinity interactions. A wide range of different non-immunoglobulin scaffolds with diverse origins and characteristics are currently used for, for example, combinatorial library display. Exemplary non-immunoglobulin based affinity proteins include Affibodies, which is based on combinatorial protein engineering of the small and robust a-helical structure of protein A. Affibodies have been widely as selective binding reagents. In addition, affibodies labelled with fluorescence markers allow quantitative measurements of non-labelled target molecules.

Exemplary non-immunoglobulin scaffolds include an antibody substructure, minibody, adnectin, anticalin, affibody, affilin, DARPin, knottin, glubody, C-type lectin-like domain protein, tetranectin, kunitz domain protein, thioredoxin, cytochrome b562, zinc finger scaffold, Staphylococcal nuclease scaffold, fibronectin or fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD 1, C2 and I-set domains of VCAM-1,1-set immunoglobulin domain of myosin-binding protein C, 1-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin.

### VIII. Small Molecules

The compositions of the present application also include small molecules, which may activate the activity of proteins with enzymatic function, and/or the interactions of said proteins. Chemical agents, referred to in the art as “small molecule” compounds are typically organic, non-peptide molecules, having a molecular weight less than 10,000, less than 5,000, less than 1,000, or less than 500 daltons. This class of activators includes chemically synthesized molecules, for instance, compounds from combinatorial chemical libraries. Synthetic compounds may be rationally designed or identified based on known or inferred properties of the ANKRD16 protein or may be identified by screening compound libraries. Alternative appropriate activators of this class are natural products, particularly secondary metabolites from organisms such as plants or fungi, which can also be identified by screening compound libraries for ANKRD16-activating activity. Methods for generating and obtaining compounds are well known in the art (Schreiber S L, Science 151: 1964-1969(2000); Radmann J. and Gunther J., Science 151: 1947-1948 (2000)). In certain embodiments, small molecules bind a portion of ANKRD16. In certain embodiments, the small molecule binds a specific isoform of ANKRD16.

As described in Example 4, ANKRD16 protects sti/sti embryonic fibroblasts from death induced by the non-cognate amino acid, serine; sti/sti cells carrying CAST-derived ANKRD16 from our congenic line, were completely rescued from cell death induced by serine.

Embodiments of the invention provide for an in vitro screening assay for identifying agents (e.g. small molecules, nucleic acids, or peptides) that protect against cell death comprising: (a) providing sticky mutant cells; (b) contacting cells in culture with an agent; (c) assaying cell death in increasing concentrations of a non-cognate amino acid (e.g. serine); and (d) comparing the results to control treated cells, wherein an agent that decreases cell death protects against cell death. In certain embodiments, the sticky mutant cells used in the screening assay express ANKRD16 protein. In one embodiment, the sticky mutant cells express low levels of ANKRD16 protein. A decrease in cell death can be determined by monitoring a decreased cell death (e.g. using viability dyes), monitoring a decrease in apoptosis, monitoring an increased cell viability, and/or monitoring improved cell functionality (e.g. Ca-channel activity, redox-potential, enzyme activity, and cell motility).

Agents that protect against cell death can a) upregulate the mRNA or protein expression of ANKRD16, b) increase the half-life of ANKRD16 RNA or protein and/or c) decrease the turnover or degradation of ANKRD16 RNA or protein.

Embodiments of the invention also provide in vitro screening assays for identifying agents (e.g. small molecules, nucleic acids, peptides or proteins) that are protectors against cell death, or activators of ANKRD16, through monitoring of ANKRD16 expression in sticky mutant cells and comparing the results to control treated cells, wherein an agent that decreases cell death protects against cell death. An agent that increases expression of ANKRD16 is an activator of ANKRD16.

In one embodiment, an in vitro assay for identifying agents that protect against cell death is provided that comprises: (a) providing sticky mutant cells; (b) contacting cells in culture with an agent; (c) assaying the expression of ANKRD16 expression (direct or indirect); and (d) comparing the results to control treated cells. An agent that increases ANKRD16 expression as compared to control treated cells protects against cell death. In one embodiment, the sticky mutant cells express low levels of ANKRD16 protein.

In certain embodiments, an in vitro assay for identifying agents that activate ANKRD16 is provided. The method comprises (a) providing sticky mutant cells that express low level of ANKRD16 protein; (b) contacting cells in culture with an agent; (c) assaying cell death in increasing concentrations of non-cognate amino acid (e.g. serine); (d) assaying the expression of ANKRD16 expression (direct or indirect); and (e) comparing the results to control treated cells. An agent that decreases cell death and increases ANKRD16 expression as compared to control treated cells is an activator of ANKRD16.

In certain embodiments, an in vitro assay for identifying agents that activate ANKRD16 is provided. The method comprises (a) providing sticky mutant cells that express low level of ANKRD16 protein; (b) contacting cells in culture with an agent; (c) assaying cell death in increasing concentrations of non-cognate amino acid (e.g. serine); and (d) comparing the results to control treated cells. An agent that decreases cell death as compared to control treated cells is an activator of ANKRD16.

An increase in ANKRD16 expression can be monitored by RNA expression (e.g. quantitative PCR) or protein expression (e. g. Western Blot, ELISA). Further, ANKRD16 expression can be monitored using reporter constructs in the cells (e.g. fluorescence, bioluminescence, and positron emission tomography (PET) using GFP, GFP derivatives, blue fluorescent protein (EBFP, EBFP2, Azurite, mKalama1), cyan fluorescent protein (ECFP, Cerulean, CyPet), yellow fluorescent protein derivatives (YFP, Citrine, Venus, YPet), beta-galactosidase, luciferase, chloramphenicol acetyltransferase, alkaline phosphatase).

Cells can be cultured and treated with compounds and assayed for a change in cell death (e.g. using viability dyes), apoptosis, cell viability (e.g. WST-1 assay, ATP assay, CellTiter-Blue by Promega, MTT assay), and/or cell functionality (e.g. Ca-channel activity, redox-potential, enzyme activity, cell motility).

Sticky mutant cells that can be used in the screening assay include, but are not limited to cells from the following mouse strains: Mice lacking the ANKRD16 gene (ANKRD16 −/−) intercrossed to sticky; mice heterozygous for the ANKRD16 gene (ANKRD16 +/−) intercrossed to sticky; mice carrying the CAST ANKRD16 allele on the sticky background (ANKRD16^{CAST/+}; sti/sti) and mice carrying the sticky mutation (sti/sti) (The sticky mouse is also called B6.Cg-Aarssti/J; available from The Jackson Laboratory Stock Number 002560). Many different cell types can be isolated from adult mice or mouse embryos. For example, embryonic fibroblast can be isolated from day 12 to day 14 embryos according to standard protocols, for example see Hogan, B. Manipulating the Mouse Embryo: A Laboratory Manual (Cold Spring Harbor Laboratory Press, Cold Spring Harbor, N.Y., 1986) or neuronal cells from embryonic brains. From adult tissues a whole variety of cells can be isolated, cultured and used for compound screening. Such cell types include fibroblasts, neuronal cells, glia cells (such as, for example, oligodendrocytes, Schwann cells), Purkinje cells, astrocytes, dorsal root ganglia, myocytes, keratinocytes, melanocytes. hepatocytes, Kupffer cells, Ito cells, stellate cells, renal tubule cells, epithelial cells, stromal cells, chondrocytes, bone cells (such as, for example, osteoblasts, osteocytes, osteoclasts), adipocytes, islet cells, endoderm-derived cells, myocytes, cardiomyocytes, smooth muscle cells, cornea cells, epithelial cells (such as, for example, mammary, bronchial, or prostate), connective tissue cells, epidermal cells, endocrine cells, lung cells, urogenital cells, cardiac, digestive tract cells, oocyte. Further such cells, could be stem cells such as, for example, mesenchymal stem cell, hematopoietic stem cell, neuronal stem cell, cord blood-derived stem cell, fat-derived stem cell, muscle-derived stem cell, skin-derived stem cells, spermatogonial stem cells, epithelial stem cells, keratinocyte stem cells) or induced pluripotent stem cells (Takahashi and Yamanaka, Cell. 2006 Aug. 25; 126(4):652-5). In certain embodiments, the cells are homozygous for the sticky mutation.

Also provided is an in vitro assay for identifying agents that activate ANKRD16 expression that comprises a) providing cells; b) contacting cells in culture with an agent; c) assaying the expression of ANKRD16 expression (direct or indirect); and d) comparing the results to control treated cells. An agent that increases ANKRD16 expression as compared to control treated cells activates ANKRD16 expression. Any cells can be used in the screening. In one embodiment, the cells are sticky mutant cells.

In one embodiment, an in vitro assay for identifying agents that activate ANKRD16 expression is provided that comprises: (a) providing cells that comprise a reporter gene operably linked to an ANKRD16 promoter; (b) contacting cells in culture with an agent; (d) assaying the reporter gene expression; and (c) comparing the results to control treated cells. An agent that has increased reporter gene expression as compared to the control treated cells activates ANKRD16 expression. Alternatively, the ANKRD16 gene is linked to a reporter gene using an internal ribosome entry site (IRES) allowing the expression of ANKRD16 and the reporter gene simultaneously. Any cells can be used in the screening. In one embodiment, the cells are sticky mutant cells. The reporter construct can include, for example, fluorescence, bioluminescence, and positron emission tomography (PET) using for example GFP, GFP derivatives, blue fluorescent protein (EBFP, EBFP2, Azurite, mKalama1), cyan fluorescent protein (ECFP, Cerulean, CyPet), yellow fluorescent protein derivatives (YFP, Citrine, Venus, YPet), beta-galactosidase, luciferase, chloramphenicol acetyltransferase, alkaline phosphatase, and the like. Further

As described above, agents (compounds) that can be screened include nucleic acids, microRNAs, siRNA, shRNAs, oligonucleotides, proteins, peptides, peptidomimetics, antibodies, antibody fragments, non-immuglobulin antigen binding scaffolds, and small molecules. Corresponding combinatorial libraries for these are well known in the art.

In one embodiment, the screening assay can be set up to with cells from sti/sti×ANKRD16 +/− mice (these are sticky mice which were intercrossed with ANKRD16-null mouse) and to screen for compounds which would increase the expression of ANKRD16.

Embodiments of the invention also provide for in vivo screening of agents that protect against cell death using ANKRD16 Knockout Mice (homozygous or heterozygous) intercrossed in to the sticky mutation (B6.Cg-Aarssti/J; available from The Jackson Laboratory Stock Number 002560). Either the whole mouse or organ cultures or organotypic slice culture can be used. The in vivo method of identifying agents that protect against cell death comprises: (a) providing a mouse that is an ANKRD16 knockout mouse intercrossed with a sticky mutant mouse; (b) administering to said mouse an agent; (c) assaying neuronal cell function in the mouse of step (b); and (d) comparing the neuronal cell function of the mouse of step (b) with the neuronal cell function of a control mouse, where an enhanced neuronal cell function as compared to the neuronal cell function of the control is indicative of an agent that protects against cell death.

Means for assaying neuronal cell function in a mouse include behavioral tests which assay neuronal cell functionality. For example Purkinje cell death results in the sticky mutants result in tremors which progresses to ataxia (Lee et al. 2006). Such behavioral tests may include sensitivity to pain, and tests which address motor, sensory, and reflex. Such assays include the rotarod assay, grip strength assay, body suspension test, open field test, light/dark test, foot print analysis, von Frey-hair pinch test (see Meta and Schwab 2004), water maze test, locomotor activity assays. Histopathological analysis of tissues sections the integrity of the neuronal cells can also be analyzed by morphological features and specific markers, using in situ hybridization and/or immunohistochemistry.

In an alternative embodiment, the assay can measure survival time, where an increase in survival time in the presence of the agent as compared to the control mouse is indicative of an agent that protects against cell death.

### IX Diagnostic Methods

Methods of detecting the level of ANKRD16 in bodily fluids include contacting a component of a bodily fluid with a ANKRD16-specific probe bound to solid matrix, e.g., microtiter plate, bead, dipstick. For example, the solid matrix is dipped into a patient-derived sample of a bodily fluid, washed, and the solid matrix contacted with a reagent to detect the presence of immune complexes present on the solid matrix. Proteins in a test sample are immobilized on (bound to) a solid matrix. Methods and means for covalently or noncovalently binding proteins to solid matrices are known in the art. The nature of the solid surface may vary depending upon the assay format. For assays carried out in microtiter wells, the solid surface is the wall of the well or cup. For assays using beads, the solid surface is the surface of the bead. In assays using a dipstick (i.e., a solid body made from a porous or fibrous material such as fabric or paper) the surface is the surface of the material from which the dipstick is made. Examples of useful solid supports include nitrocellulose (e.g., in membrane or microtiter well form), polyvinyl chloride (e.g., in sheets or microtiter wells), polystyrene latex (e.g., in beads or microtiter plates, polyvinylidine fluoride (known as IMMULON™), diazotized paper, nylon membranes, activated beads, and Protein A beads. Microtiter plates may be activated (e.g., chemically treated or coated) to covalently bind proteins. The solid support containing the probe is typically washed after contacting it with the test sample, and prior to detection of bound immune complexes.

In certain embodiments of these assays is that an ANKRD16-specific binding moiety is contacted with a sample of bodily fluid under conditions that permit ANKRD16 to bind to the antibody forming an immune complex containing the patient ANKRD16 bound to an ANKRD16-specific antibody. Such conditions are typically physiologic temperature, pH, and ionic strength. The incubation of the antibody with the test sample is followed by detection of immune complexes by a detectable label. For example, the label is enzymatic, fluorescent, chemiluminescent, radioactive, or a dye. Assays which amplify the signals from the immune complex are also known in the art, e.g., assays which utilize biotin and avidin.

Antibodies and nucleic acid compositions disclosed herein are useful in diagnostic and prognostic evaluation of neurodegenerative disease or a disease caused by misfolded protein, associated with ANKRD16 expression.

Methods of diagnosis can be performed in vitro using a cellular sample (e.g., blood serum, plasma, urine, saliva, cerebral spinal fluid, joint fluid, fluid from the pleural space, peritoneal fluid, lymph node biopsy or tissue) from a patient or can be performed by in vivo imaging. In certain embodiments, samples may comprise neuronal cells.

In particular embodiments, the present application provides an antibody conjugate wherein the antibodies of the present application are conjugated to a diagnostic imaging agent. Compositions comprising the antibodies of the present application can be used to detect ANKRD16, for example, by radioimmunoassay, ELISA, FACS, immunohistochemistry, Western blot etc. One or more detectable labels can be attached to the antibodies. Exemplary labeling moieties include radiopaque dyes, radiocontrast agents, metals (e.g., gold), fluorescent molecules, spin-labeled molecules, enzymes, or other labeling moieties of diagnostic value, particularly in radiologic or magnetic resonance imaging techniques.

In certain embodiments, tissue sections are stained or the tissue is disrupted, e.g., homogenized, and processed as for a fluid. ANKRD16 is quantified by methods known in the art, e.g., immunoblot analysis followed by densitometry or immunohistochemistry staining and quantitative scoring of ANKRD16 isoforms in the sections. In certain embodiments, a specific isoform of ANKRD16 is evaluated. In certain embodiments, the ratio of full length to short form is calculated and evaluated.

A radiolabeled antibody in accordance with this disclosure can be used for in vitro diagnostic tests. The specific activity of an antibody, binding portion thereof, probe, or ligand, depends upon the half-life, the isotopic purity of the radioactive label, and how the label is incorporated into the biological agent. In immunoassay tests, the higher the specific activity, in general, the better the sensitivity. Radioisotopes useful as labels, e.g., for use in diagnostics, include iodine (¹³¹I or ¹²⁵I), indium (¹¹¹In), technetium (⁹⁹Tc), phosphorus (³²P), carbon (¹⁴C), and tritium (³H), or one of the therapeutic isotopes listed above.

The radiolabeled antibody can be administered to a patient where it is localized to cells bearing the antigen with which the antibody reacts, and is detected or “imaged” in vivo using known techniques such as radionuclear scanning using e.g., a gamma camera or emission tomography. See e.g., Bradwell et al., “Developments in Antibody Imaging”, Monoclonal Antibodies for Cancer Detection and Therapy, Baldwin et al., (eds.), pp. 65-85 (Academic Press 1985), which is hereby incorporated by reference. Alternatively, a positron emission transaxial tomography scanner, such as designated Pet VI located at Brookhaven National Laboratory, can be used where the radiolabel emits positrons (e.g., ₁₁C, ¹⁸F, ¹⁵O, and ¹³N).

Fluorophore and chromophore labeled biological agents can be prepared from standard moieties known in the art. Since antibodies and other proteins absorb light having wavelengths up to about 310 nm, the fluorescent moieties may be selected to have substantial absorption at wavelengths above 310 nm, such as for example, above 400 nm. A variety of suitable fluorescers and chromophores are described by Stryer, Science, 162:526 (1968) and Brand et al., Annual Review of Biochemistry, 41:843-868 (1972), which are hereby incorporated by reference. The antibodies can be labeled with fluorescent chromophore groups by conventional procedures such as those disclosed in U.S. Pat. Nos. 3,940,475, 4,289,747, and 4,376,110, which are hereby incorporated by reference.

In certain embodiments, antibody conjugates or nucleic acid compositions for diagnostic use in the present application are intended for use in vitro, where the composition is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. In certain embodiments, secondary binding ligands are biotin and avidin or streptavidin compounds.

In certain embodiments, SNPs in ANKRND16 such as those described in the examples may be examined for diagnostic of prognostic methods of the application. In certain embodiments, SNPs in ANKRND16 may be associated with increased or decreased ANKRND16 activity. In certain embodiments, SNPs in ANKRND16 may be associated with changes in the expression of ANKRND16 isoforms. In certain embodiments, SNPs in ANKRND16 may be associated with a good or poor prognosis. In certain embodiments, SNPs in ANKRND16 may be associated with a neurodegenerative disease or a disease caused by misfolded proteins. Polymorphism can be detected by other commonly used genotyping methods, such as PCR techniques, nucleic acid sequencing, microsatellite markers, ligase chain reaction, nucleic acid hybridization techniques, whole genome hybridization, microarray and microsphere assays. Especially the identification of novel stop codons or novel splice sites leading to truncated forms of ANKRD16 or the loss of ANKRD16 is useful as prognostic or diagnostic.

In certain embodiments the diagnostic methods of the application may be used in combination with other neurodegenerative disease diagnostic tests.

The present application also provides for a diagnostic kit comprising anti-ANKRD16 antibodies or nucleic acid compositions that bind at least one isoform of ANKRD16. Such a diagnostic kit may further comprise a packaged combination of reagents in predetermined amounts with instructions for performing the diagnostic assay. Where the antibody is labeled with an enzyme, the kit will include substrates and co-factors required by the enzyme. In addition, other additives may be included such as stabilizers, buffers and the like. The relative amounts of the various reagents may be varied widely to provide for concentrations in solution of the reagents that substantially optimize the sensitivity of the assay. Particularly, the reagents may be provided as dry powders, usually lyophilized, including excipients that, on dissolution, will provide a reagent solution having the appropriate concentration.

In another aspect, the present application concerns immunoassays for binding, purifying, quantifying and otherwise generally detecting ANKRD16 protein components. As detailed below, immunoassays, in their most simple and direct sense, are binding assays. In certain embodiments, immunoassays are the various types of enzyme linked immunoadsorbent assays (ELISAs) and radioimmunoassays (RIA) known in the art. Immunohistochemical detection using tissue sections is also particularly useful. However, it will be readily appreciated that detection is not limited to such techniques, and Western blotting, dot and slot blotting, FACS analyses, and the like may also be used.

The steps of various useful immunoassays have been described in the scientific literature, such as, e.g., Nakamura et al., in Enzyme Immunoassays: Heterogeneous and Homogeneous Systems, Chapter 27 (1987), incorporated herein by reference.

In general, the immunobinding methods include obtaining a sample suspected of containing a protein or peptide, in this case, ANKRD16 and contacting the sample with a first antibody immunoreactive with ANKRD16 under conditions effective to allow the formation of immunocomplexes.

Immunobinding methods include methods for purifying ANKRD16 proteins, as may be employed in purifying protein from patients' samples or for purifying recombinantly expressed protein. They also include methods for detecting or quantifying the amount of ANKRD16 in a tissue sample, which requires the detection or quantification of any immune complexes formed during the binding process.

The biological sample analyzed may be any sample that is suspected of containing ANKRD16 such as a homogenized tissue sample. Contacting the chosen biological sample with the antibody under conditions effective and for a period of time sufficient to allow the formation of primary immune complexes) is generally a matter of adding the antibody composition to the sample and incubating the mixture for a period of time long enough for the antibodies to form immune complexes with, i.e., to bind to, any ANKRD16 present. The sample-antibody composition is washed extensively to remove any non-specifically bound antibody species, allowing only those antibodies specifically bound within the primary immune complexes to be detected.

In general, the detection of immunocomplex formation is well known in the art and may be achieved through the application of numerous approaches. These methods are based upon the detection of radioactive, fluorescent, biological or enzymatic tags. Of course, one may find additional advantages through the use of a secondary binding ligand such as a second antibody or a biotin/avidin ligand binding arrangement, as is known in the art.

The anti-ANKRD16 antibody used in the detection may itself be conjugated to a detectable label, wherein one would then simply detect this label. The amount of the primary immune complexes in the composition would, thereby, be determined.

Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are washed extensively to remove any non-specifically bound labeled secondary antibodies or ligands, and the remaining label in the secondary immune complex is detected.

An enzyme linked immunoadsorbent assay (ELISA) is a type of binding assay. In one type of ELISA, anti-ANKRD16 antibodies used in the diagnostic method of this application are immobilized onto a selected surface exhibiting protein affinity, such as a well in a polystyrene microtiter plate. Then, a suspected neoplastic tissue sample is added to the wells. After binding and washing to remove non-specifically bound immune complexes, the bound ANKRD16 may be detected. Detection is generally achieved by the addition of another anti-ANKRD16 antibody that is linked to a detectable label. This type of ELISA is a simple “sandwich ELISA.” Detection may also be achieved by the addition of a second anti-ANKRD16 antibody, followed by the addition of a third antibody that has binding affinity for the second antibody, with the third antibody being linked to a detectable label.

In another type of ELISA, the tissue samples are immobilized onto the well surface and then contacted with the anti-ANKRD16 antibodies used in this application. After binding and washing to remove non-specifically bound immune complexes, the bound anti-ANKRD16 antibodies are detected. Where the initial anti-ANKRD16 antibodies are linked to a detectable label, the immune complexes may be detected directly. Alternatively, the immune complexes may be detected using a second antibody that has binding affinity for the first anti-ANKRD16 antibody, with the second antibody being linked to a detectable label.

Irrespective of the format employed, ELISAs have certain features in common, such as coating, incubating or binding, washing to remove non-specifically bound species, and detecting the bound immune complexes.

The radioimmunoassay (RIA) is an analytical technique which depends on the competition (affinity) of an antigen for antigen-binding sites on antibody molecules. Standard curves are constructed from data gathered from a series of samples each containing the same known concentration of labeled antigen, and various, but known, concentrations of unlabeled antigen. Antigens are labeled with a radioactive isotope tracer. The mixture is incubated in contact with an antibody. Then the free antigen is separated from the antibody and the antigen bound thereto. Then, by use of a suitable detector, such as a gamma or beta radiation detector, the percent of either the bound or free labeled antigen or both is determined. This procedure is repeated for a number of samples containing various known concentrations of unlabeled antigens and the results are plotted as a standard graph. The percent of bound tracer antigens is plotted as a function of the antigen concentration. Typically, as the total antigen concentration increases the relative amount of the tracer antigen bound to the antibody decreases. After the standard graph is prepared, it is thereafter used to determine the concentration of antigen in samples undergoing analysis.

In an analysis, the sample in which the concentration of antigen is to be determined is mixed with a known amount of tracer antigen. Tracer antigen is the same antigen known to be in the sample but which has been labeled with a suitable radioactive isotope. The sample with tracer is then incubated in contact with the antibody. Then it can be counted in a suitable detector which counts the free antigen remaining in the sample. The antigen bound to the antibody or immunoadsorbent may also be similarly counted. Then, from the standard curve, the concentration of antigen in the original sample is determined.

In certain embodiments, immunocytochemical techniques are used as follows. Cells or tissue sections are processed for labeling. For example, cells are plated on chamber slides for 24 h, then serum deprived for another 24 h. Cells are rinsed with PBS and fixed in 4% paraformadehyde for 20 min. Cells are then treated with 1% Triton X-100 for 10 min. followed by incubation with preimmune serum in PBS to block non-specific binding. Primary antibodies are diluted in 3% normal donkey serum and incubated with cells for 1 h. Finally fluorescent labeled secondary antibodies are applied for 30 min. Slides are examined by fluorescence microscopy.

In certain embodiments, Enzyme-linked Immunosorbent assay (ELISA) is used to evaluate ANKRD16 protein level.

In certain embodiments a bead assay is used to evaluate ANKRD16 protein level. This assay can be part of a multiplex platform, such as Luminex or magnetic beads.

In certain embodiments, Western immunoblot analysis is carried out using well known methods. For example, cells or tissues were solubilized or homogenized in RIPA buffer at 4° C. for 20 min. The supernatants are collected after centrifugation at 13,000×g for 10 min at 4° C. Protein concentration is determined using a bicinchoninic acid (BCA) protein assay kit (Sigma). Samples of equal amount of protein were mixed with Laemmli's sample buffer, fractionated by 7.5-15% SDS-polyacrylamide gels under reducing condition, and transferred to nitrocellulose membrane. The membrane is probed with specific antibodies. The blots are developed using an enhanced chemiluminescence system (Amersham).

### X Methods of Treatment

In certain embodiments, the present disclosure provides methods of activating ANKRD16 and methods of treating neurodegenerative diseases or diseases caused by misfolded proteins. In certain embodiments, ANKRD16 can be utilized as a cell-protective protein including as a neuroprotective. These methods involve administering to the individual a therapeutically effective amount of one or more therapeutic agents as described above. These methods are particularly aimed at therapeutic and prophylactic treatments of animals, and more particularly, humans.

Neurodegenerative diseases include, but are not limited to, Alexander disease, Alper's disease, Alzheimer's disease, Amyotrophic lateral sclerosis, Ataxia telangiectasia, Batten disease, Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease, Multiple sclerosis, Multiple System Atrophy, Parkinson disease, Pelizaeus-Merzbacher Disease, Pick's disease, Primary lateral sclerosis, Prion diseases, Refsum's disease, Sandhoff disease, Schilder's disease, Schizophrenia, Spielmeyer-Vogt-Sjogren-Batten disease, Spinocerebellar ataxia, Spinal muscular atrophy, Steele-Richardson-Olszewski disease, or Tabes dorsalis.

The present disclosure also provides methods for treating proteopathies. Proteopathy diseases or disorders are associated with the abnormal accumulation of proteins. The protopathies, sometimes referred to as proteinopathies include more than 30 diseases and disorders that affect a variety of organs and tissue. Examples of protopathies include, but are not limited to, Type II diabetes, infertility, reduced fertility, cancers, such as thyroid carcinoma, Aortic medial amyloidosis, ApoAI amyloidosis, ApoAII amyloidosis, ApoAIV amyloidosis, Finnish hereditary amyloidosis, Lysozyme amyloidosis, Fibrinogen amyloidosis, Dialysis amyloidosis, Inclusion body myopathy/myositis, Cataracts, Medullary thyroid carcinoma, Cardiac atrial amyloidosis, Pituitary prolactinoma, Hereditary lattice corneal dystrophy, Cutaneous lichen amyloidosis, Corneal lactoferrin amyloidosis, Pulmonary alveolar proteinosis, Critical illness myopathy (CIM).

Methods for treating diseases caused by misfolded proteins are also provided and include, but are not limited to, cystic fibrosis, mad cow disease, hereditary forms of emphysema caused my misfolding of P22 tailspike protein, certain forms of cancer caused by misfolding of p53, and another diseases caused by misfolded proteins.

In certain embodiments of such methods, one or more therapeutic agents can be administered, together (simultaneously) or at different times (sequentially). In addition, therapeutic agents can be administered with another type of compounds for treating neurodegenerative diseases, diseases caused by misfolded proteins, or proteopathies.

Depending on the nature of the combinatory therapy, administration of the therapeutic agents of the disclosure may be continued while the other therapy is being administered and/or thereafter. Administration of the therapeutic agents may be made in a single dose, or in multiple doses. In some instances, administration of the therapeutic agents is commenced at least several days prior to the conventional therapy, while in other instances, administration is begun either immediately before or at the time of the administration of the conventional therapy.

### XI. Methods of Administration and Pharmaceutical Compositions

In certain embodiments, the subject compositions of the present disclosure are formulated with a pharmaceutically acceptable carrier. Such therapeutic agents can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the subject agents include those suitable for oral/nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

In certain embodiments, methods of preparing these formulations or compositions include combining another type of neurodegenerative disease or a disease caused by misfolded protein therapeutic agent and a carrier and, optionally, one or more accessory ingredients. In general, the formulations can be prepared with a liquid carrier, or a finely divided solid carrier, or both, and then, if necessary, shaping the product.

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a subject therapeutic agent as an active ingredient.

In solid dosage forms for oral administration (capsules, tablets, pills, dragees, powders, granules, and the like), one or more therapeutic agents of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof

In particular, methods of the disclosure can be administered topically, either to skin or to mucosal membranes such as those on the cervix and vagina. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The subject agents may be mixed under sterile conditions with a pharmaceutically acceptable carrier, and with any preservatives, buffers, or propellants which may be required. The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof

Powders and sprays can contain, in addition to a subject therapeutic agent, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more therapeutic agents in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsulated matrices of one or more therapeutic agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Formulations for intravaginal or rectally administration may be presented as a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Methods for delivering the subject nucleic acid compounds are known in the art (see, e.g., Akhtar et al., 1992, Trends Cell Bio., 2, 139; and Delivery Strategies for Antisense Oligonucleotide Therapeutics, ed. Akhtar, 1995; Sullivan et al., PCT Publication No. WO 94/02595). These protocols can be utilized for the delivery of virtually any nucleic acid compound. Nucleic acid compounds can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to, oral (tablet or pill form) and/or intrathecal delivery (Gold, 1997, Neuroscience, 76, 1153-1158). Other approaches include the use of various transport and carrier systems, for example though the use of conjugates and biodegradable polymers. For a comprehensive review on drug delivery strategies, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. Neuro Virol., 3, 387-400. More detailed descriptions of nucleic acid delivery and administration are provided in Sullivan et al., supra, Draper et al., PCT WO93/23569, Beigelman et al., PCT Publication No. WO99/05094, and Klimuk et al., PCT Publication No. WO99/04819.

In certain embodiments, the nucleic acids of the instant disclosure are formulated with a pharmaceutically acceptable agent that allows for the effective distribution of the nucleic acid compounds of the instant disclosure in the physical location most suitable for their desired activity. Non-limiting examples of such pharmaceutically acceptable agents include: PEG, phospholipids, phosphorothioates, P-glycoprotein inhibitors (such as Pluronic P85) which can enhance entry of drugs into various tissues, biodegradable polymers, such as poly (DL-lactide-coglycolide) microspheres for sustained release delivery after implantation (Emerich, D F et al, 1999, Cell Transplant, 8, 47-58), and loaded nanoparticles such as those made of polybutylcyanoacrylate, which can deliver drugs across the blood brain barrier and can alter neuronal uptake mechanisms (Prog Neuropsychopharmacol Biol Psychiatry, 23, 941-949, 1999).

In other embodiments, certain of the nucleic acid compounds of the instant disclosure can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45). Those skilled in the art realize that any nucleic acid can be expressed in eukaryotic cells from the appropriate DNA/RNA vector. The activity of such nucleic acids can be augmented by their release from the primary transcript by an enzymatic nucleic acid (Draper et al, PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al., 2000, Cent. Nerv. Syst. Dis., 485-508; Peel and Klein, 2000, J. Neurosci. Methods, 98, 95-104; Hagihara et al., 2000, Gene Ther., 7, 759-763; and Herrlinger et al., 2000, Methods Mol. Med., 35, 287-312. AAV-mediated delivery of nucleic acid to cells of the nervous system is further described by Kaplitt et al., U.S. Pat. No. 6,180,613.

In another aspect of the disclosure, RNA molecules of the present disclosure are preferably expressed from transcription units (see for example Couture et al., 1996, TIG., 12, 510) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid compounds are delivered as described above, and persist in target cells. Alternatively, viral vectors can be used that provide for transient expression of nucleic acid compounds. Such vectors can be repeatedly administered as necessary. Once expressed, the nucleic acid compound binds to the target mRNA. Delivery of nucleic acid compound expressing vectors can be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

In one aspect, the disclosure contemplates an expression vector comprising a nucleic acid sequence encoding at least one of the nucleic acid compounds of the instant disclosure. The nucleic acid sequence is operably linked in a manner which allows expression of the nucleic acid compound of the disclosure. For example, the disclosure features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant disclosure; and wherein said sequence is operably linked to said initiation region and said termination region, in a manner which allows expression and/or delivery of said nucleic acid compound. The vector can optionally include an open reading frame (ORF) for a protein operably linked on the 5′ side or the 3′-side of the sequence encoding the nucleic acid catalyst of the disclosure; and/or an intron (intervening sequences).

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Identification of ANKRD16 as a Suppressor Gene of Neuron Death in sti Mutant Mice

Performing an intersubspecific intercross of the sticky (sti) mutation homozygous on C57BL/6J (B6) background (B6.stock-sti/sti; B6.Cg-Aarssti/J: available from The Jackson Laboratory Stock number (002560)) with CAST/Ei (Mus musculus castaneus; available from The Jackson Laboratory (Stock number 00928)). The resulted in the generation of F1 sti/+mice. Then the F1 mice were intercrossed to generate 1466 F2 animals for recombination mapping. Analyzing the sti/sti F2 animals from our B6 X CAST/Ei intersubspecific mapping cross revealed that most of the sti/sti F2 mice did not develop ataxia and had little Purkinje cell loss, even when aged to 16 months. The candidate modifier gene was mapped to chromosome 2 (Chr 2) using standard genetic tools, e.g. backcrossing and genome scans. Initial genome scans on 70 N1 backcross mice suggested that the modifier gene derived from the Cast/Ei (CAST) genome was localized in a 4 cM region between D2Mit4 and D2Mit427 on proximal Chr 2 and acted in a dominant fashion. Use of congenic mice and further backcrossing as known in the art revealed that animals heterozygous for the CAST-derived region on Chr 2 were not ataxic. Histopathological analysis of the cerebellum using immunohistochemistry with antibodies to calbindin D-28 revealed this region on Chr 2 is in fact sufficient to protect most Purkinje cells from sti-mediated cell death (data not shown) even aged to 1 year. Comparing calbindin D-28 stained brain sections of B6.CAST D2Mit4-D2Mit427-sti/sti (B6.Stim^{CAST}) mice at 3 months of age to such of B6 mice showed almost no difference. Accordingly, CAST proximal Chr 2 suppresses sti-mediated Purkinje cell death. The immunohistochemistry for to calbindin D-28 was performed as described in Ackerman et al. (1997), Nature 386, 838-842.

### Example 2

### Splice Variations of ANKRD16

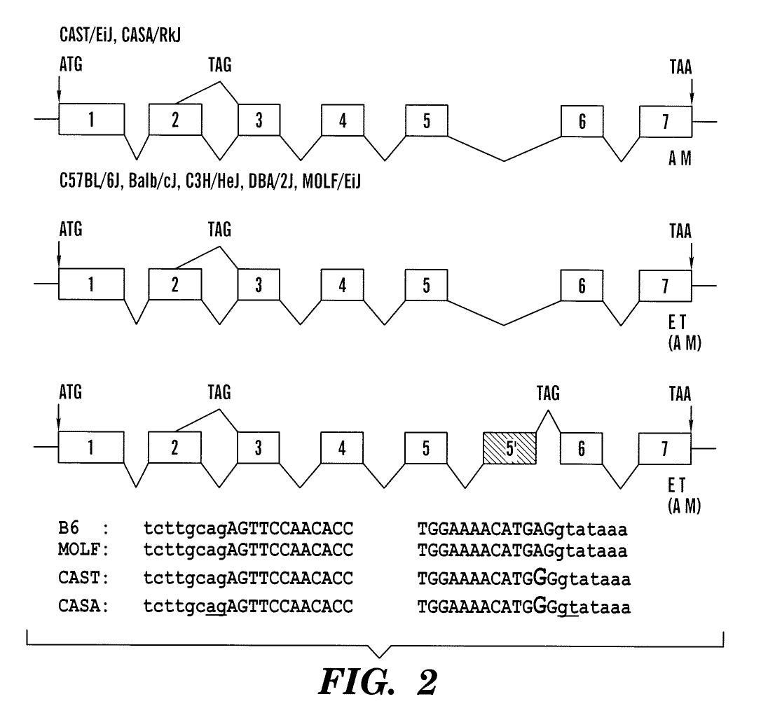

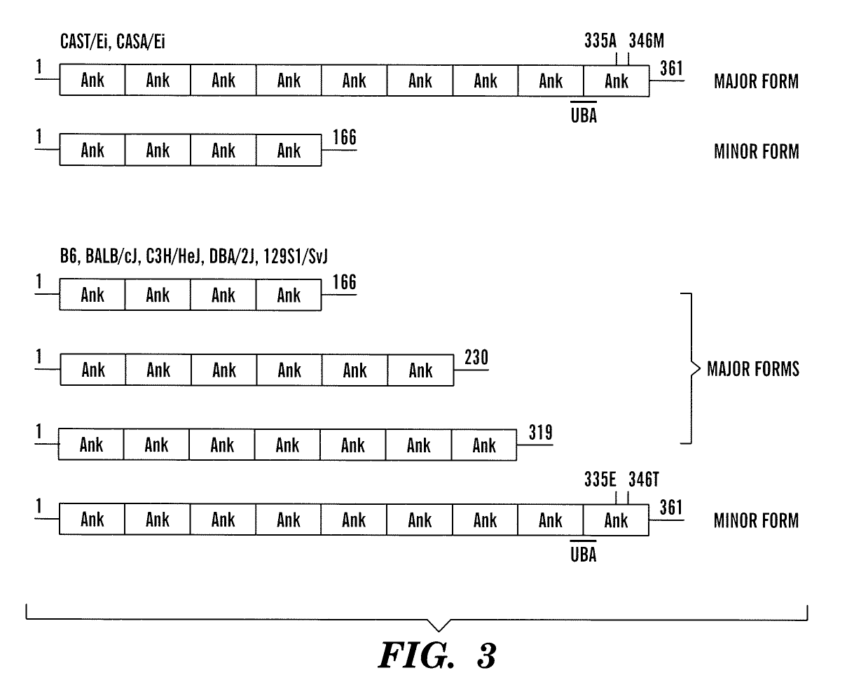

Two amino acid polymorphisms near the C-terminus occur between sti-rescuing strains (CAST/Ei and CASA/Rk with The Jackson Laboratory Stock Number 000928 and 000735 respectively) and several non-rescuing strains (C57BL/6J (B6), DBA/2J and C3H/HeJ; FIG. 2 and FIG. 3) (C57BL/6J, DBA/2J and C3H/HeJ are available from The Jackson Laboratory with the stock numbers 000664, 000671, and 000659 respectively). However, the coding region of MOLF/EiJ (a non-rescuing strain; available from the Jackson Laboratory with the stock number 000550) is predicted to encode the amino acids found in CAST/Ei, not B6, demonstrating that these polymorphisms are not solely responsible for the rescuing ability of this gene. In contrast, all non-rescuing strains (including MOLF/EiJ) demonstrate aberrant splicing of ANKRD16, which leads to multiple transcripts with in-frame translational termination codon leading to truncation of the C-terminus. CAST/Ei and CASA/ERk mice utilize only 1 of these 3 alternative splice sites and produce much higher levels of full-length transcript. Much lower levels of full-length ANKRD16 (ANKRD16³⁶¹) are produced by the non-rescuing strains. Although produced at much lower levels relative to CAST/Ei mice, correctly spliced transcripts encoding full-length ANKRD16 are found by RT-PCR in all non-rescuing mouse strains, suggesting that these strains are likely hypomorphic, not null, alleles of this gene. Interestingly, like the mouse transcript, the human ANKRD16 is also alternatively spliced, encoding both full-length and truncated forms.

FIG. 2 shows the alternative splicing of the ANKRD16 transcript in different mouse strains. In the mouse strains C57BL/6J (B6), Balb/cJ, C3H/HeJ, DBA/2J and MOLF/J the Ankrd16 mRNA is alternatively spliced when compared to CAST/EiJ and CASA/Rid mRNA. The majority of Ankrd16 mRNAs from C57BL/6J (B6), Balb/cJ, C3H/HeJ, DBA/2J and MOLF/mouse strains utilize an additional exon, called exon 5′, between exon 5 and exon 6 which leads to an early translational termination codon (labeled TAG in FIG. 2) and a shortened transcript. This transcript is unstable. Another alternative splice variant, has been observed between exon 2 and 3 in all mouse strains analyzed, which results in translational termination codon (TAG) leading to a short transcript. This transcript is unstable, likely due to nonsense-mediated decay. In CAST/EiJ and CASA/Rid mice higher amounts of full length Ankrd16 mRNA have been detected. Further, amino acid polymorphisms encoded by exon 7 are shown in FIG. 2. MOLF has the same amino acids (A,M) that CAST and CASA have suggesting these polymorphisms are not likely to be important. Note the polymorphism in exon 5′ (A to G, C57BL/6J and MOLF versus CAST/EiJ and CASA/RkJ, respectively) upstream of the splice donor (GT), which likely confers higher use of this exon in strains without the modifier.

### Example 3

### Identification of ANKRD16

We screened the CAST BAC library with probes homologous to genes between our flanking markers, as predicted from the database analysis of the B6 genome. Several overlapping clones were isolated. Analysis of these clones suggested these genes were not grossly rearranged in the CAST genome and that this region is similar in size to that of B6.

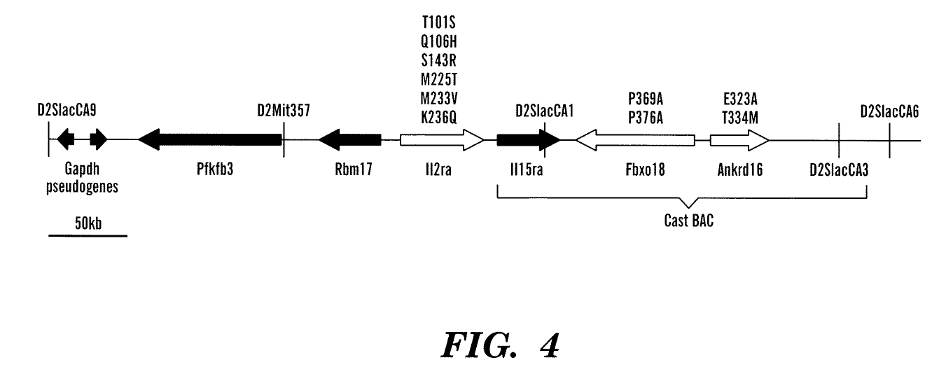

To further investigate candidate genes for function as modifier of sticky, pronuclear injection was performed with BAC FAH46 DNA. The embryos used for the injection were from the C57BL/6J strain. The BAC FAH46 contained the 3′ portion of the nonpolymorphic IL15Rα gene, and the Fbxo18 and ANKRD16 genes, which are both polymorphic between B6 and CAST. Transgenic animals were crossed to B6.sti/sti mice and the resulting F1 mice were backcrossed to B6.sti/sti mice to produce B6.sti/sti; Tgfah-46 animals. Analysis of these animals at 3 months of age demonstrated that they had a suppressed phenotype identical (by neuron counts) to that seen in the congenic CAST.B6 sti/sti mice described above in Example 1. This demonstrates that the modifier of sticky is either the Fbxo18 or ANKRD16 gene. Interestingly another transgenic line (Tgfah-19) generated with this same BAC failed to suppress Purkinje cell degeneration in sti/sti mice. Analysis of this transgenic line revealed that although the CAST allele of Fbxo18 was expressed, no expression of CAST ANKRD16 was observed. Genomic PCR using markers differentiating between CAST/Ei and B6 revealed that injected BAC in this line was deleted for the ANKRD16 gene (FIG. 4).

Analysis of ANKRD16 expression in silico (found at symatlas.gnf.org) or by in situ analysis of adult brain (data not shown) revealed ANKRD16 is ubiquitously expressed and expression is apparent in early stages of development.

### Example 4

### ANKRD16 also Protects sti/sti Embryonic Fibroblasts from Death Induced by the Non-Cognate Amino acid, Serine

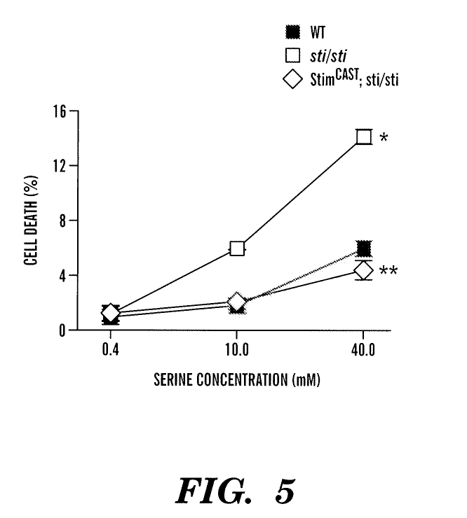

We analyzed serine-induced cell death in +/+, ANKRD16^{CAST}/+; sti/sti, and sti/sti embryonic fibroblasts. Mouse embryonic fibroblasts were isolated from mouse embryos carrying no mutation (WT; C57BL/6J), the CAST-derived ANKRD16 gene (ANKRD16^{CAST}/+) and the sticky (sti/sti) mutation, or carrying the C57BL/6 ANKRD16 gene and the sti mutation (sti/sti). Significantly more cell death was seen in sti/sti fibroblasts relative to that observed in wild type cells. However, sti/sti cells carrying CAST-derived ANKRD16 from our congenic line were completely rescued in this assay (FIG. 5). This indicates that ANKRD16 may confer protection against cell death in a variety of cell types. Furthermore, these results provide an in vitro assay to further determine the mechanism by which ANKRD16 protects against cell death.

### Example 5

### ANKRD16 Acts Cell Autonomously to Rescue Neuron Death

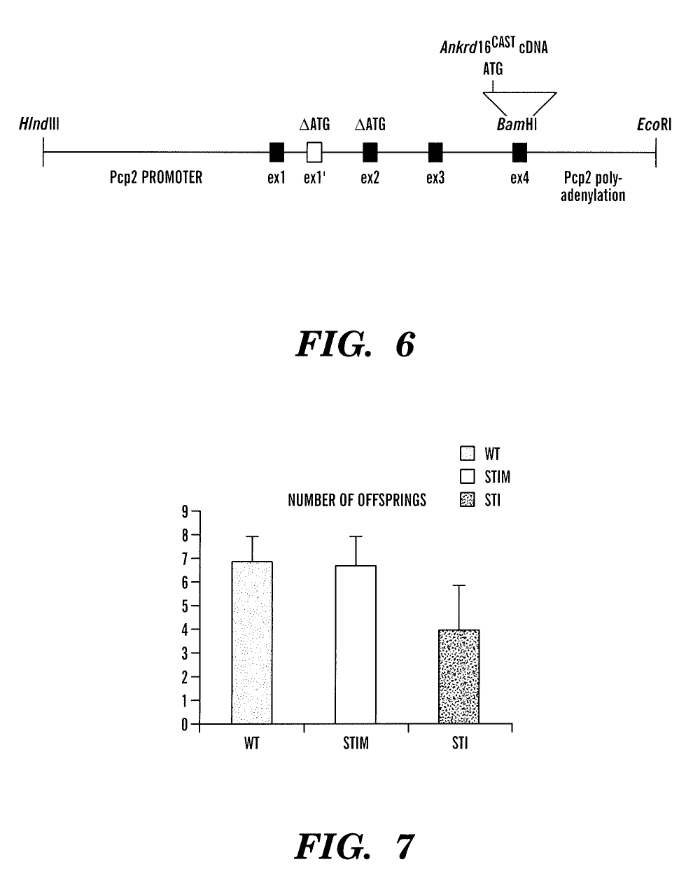

To determine if ANKRD16 can rescue neuron death in a cell autonomous fashion, we transgenically expressed the ANKRD16 cDNA in Purkinje cells and examined the role of this transgene in Purkinje cell survival in sti/sti mice. ANKRD16 coding sequence was amplified by polymerase chain reaction from cDNA by using Pfu DNA polymerase (Stratagene), purified by agarose gel electrophoresis and ligated into the expression vector. Transgenic (TgPcp2-ANKRD16) mice carrying the full-length ANKRD16 cDNA (ANKRD16³⁶¹ or CAST allele) were generated on the inbred C57BL/6J background by microinjection of the purified DNA into C57BL/6J zygotes. Expression of the transgene was driven by the Pcp2 (L7) promoter, which has been shown to express only in cerebellar Purkinje cells and bipolar cells in the retina (Oberdick J, Smeyne R J, Mann J R, Zackson S, Morgan J I. 1990. A promoter that drives transgene expression in cerebellar Purkinje and retinal bipolar neurons (see Science 248:223-226) (FIG. 6). Expression driven by this promoter begins at P0 and is detectable in all Purkinje cells by postnatal week 3. Transgenic mice were crossed to B6.sti/sti mice to generate F1 TgANKRD16; sti/+ mice and these animals were backcrossed to B6.sti/+ mice to generate F2 TgANKRD16; sti/sti mice and sti/sti mice lacking the transgene. Immunohistochemistry with antibodies to calbindin D-28 demonstrates suppression of Purkinje cell death in Pcp2-ANKRD16^{Cast} transgenic mice (data not shown). At 3 months of age, the vast majority of Purkinje cells in sti mutant cerebella had degenerated as expected, but sti/sti mice carrying the transgene showed no signs of ataxia, and the suppression of Purkinje cell degeneration was remarkably similar to that observed in the congenic CAST.B6 sti/sti and B6.sti/sti; Tgfah-46 mice described above. This result indicates that expression of ANKRD16 in sti/sti Purkinje cells is sufficient to block neuron death and Purkinje cell degeneration.

### Example 6

### Autophagy Induction Does Not Cause Relocalization of ANKRD16

Cellular localization of full-length ANKRD16 was determined by transfection of epitope-tagged full-length ANKRD16^{CAST} into monkey kidney fibroblast COS-7 cells and human alveolar epithelial A549 cells. The ANKRD16 coding sequence was amplified by polymerase chain reaction from cDNA by using Pfu DNA polymerase (Stratagene), purified by agarose gel electrophoresis and ligated in-frame with the HA-tag into the pcDNA3 vector. This clone encoded a fusion ANKRD16 with the tag at its C-terminus. All DNA constructs were confirmed by sequencing. COS-7 and A549 cells were maintained in Dulbecco's modified Eagle's medium (Sigma-Aldrich). All cells were grown at 37° C. in 5% CO2 and supplemented with 10% (vol/vol) fetal calf serum, 100 U/ml penicillin and 100 μg/ml streptomycin. Transfections were performed using Lipofectamine 2000 transfection reagent (Invitrogen, USA) according to the manufacturer's protocol. Cells were cultured for 48 h after transfection.

Diffuse expression of ANKRD16 protein was found throughout the nucleus and cytoplasm in both cell lines with all constructs tested (HA- or Flag-eptiope tagged either at the N or C terminus, EGFP- or DSRed-fusion proteins, data not shown).

To determine if the induction of autophagy changes the intracellular localization of ANKRD16, we treated transfected cells with rapamycin to induce macroautophagy (data not shown) or performed serum starvation experiments (known to induce chaperone-mediated autophagy after 18 hours) on transfected cells (data not shown). COS-7 cells were transfected with full-length HA-ANKRD16^{CAST} constructs. 24 hours post-transfection, cells were treated with 1004 rapamycin for 24 hours to induce macroautophagy, which does not alter the subcellular localization of ANKRD16. Under these conditions, the diffuse cytoplasmic and nuclear expression of ANKRD16 did not change. Nor was co-localization observed with endosome, autophagosome or lysosome markers (data not shown) in the presence or absence of rapamycin or proteasome inhibitors administered as described below.

### Example 7

### Accumulation of Misfolded Proteins Induces Relocalization of ANKRD16

Several proteins that regulate protein folding and/or the disposal of misfolded proteins are induced or change their subcellular localization in response to misfolded protein stress. To examine if subcellular localization changes with intracellular accumulation of misfolded proteins, we treated full-length HA-ANKRD16^{CAST}-transfected COS-7 cells 24 hours after transfection with proteosome inhibitors, 10 μM MG132 (Sigma-Aldrich) or 10 μM epoximicin (Sigma-Aldrich) or blocked autophagy with 3-methyladenine (3-MA; Sigma-Aldrich). Relocalization of ANKRD16 to a juxtanuclear structure was observed in the majority of transfected cells within 12 hours of treatment. Closer examination suggested ANKRD16 relocalized to the aggresome, the microtubule-based inclusion body where misfolded proteins, chaperones, and proteosome subunits accumulate in the cell (Garcia-Mata et al., 1999a). Aggresome localization of ANKRD16 under these conditions was confirmed by co-immunofluorescence with the microtubule organization center protein, γ-tubulin antibodies delineating the cellular position at which aggresomes form, HDAC-6 antibodies, a component of the aggresome, antibodies to the intermediate filament protein vimentin, which forms a cage around the aggresome, 20S proteosome subunit antibodies, and ubiquitin antibodies (data not shown). Coimmunofluorescene was performed with antibodies to ubiquitin and HA. ANKRD16 relocalized to the aggresome in proteasome-inhibitor treated cells.

Consistent with aggresome localization, the formation of this structure was disrupted by treatment of transfected cells with 10 μg/ml nocodazole (Sigma-Aldrich), which depolymerizes microtubules (data not shown). Importantly, ANKRD16 is never observed to form an aggresome like structure in the absence of proteosome or autophagy inhibitors, but was expressed in a diffuse pattern throughout the nucleus and cytoplasm.

Colocalization with misfolded proteins was also found when epitope-tagged ANKRD16 was co-transfected with constructs encoding mutant proteins that spontaneously misfold. ANKRD16 colocalized with a GFP-tagged mutant form of the cystic fibrosis transmembrane conductance regulator (CTFR-ΔF508) that forms aggresomes in the presence of proteasome inhibitors MG132-treated in COS-7 cells. (data not shown).

ANKRD16 was also co-transfected with a GFP-tagged internal segment of GCP170 (also known as golgin-160) previously shown to spontaneously accumulate in large juxtanuclear ribbon-like aggregates and spherical nuclear aggregates (Fu et al., 2005; Garcia-Mata et al., 1999a). Interestingly, strong co-localization was seen with cytoplasmic aggregates but not with nuclear inclusions (data not shown). The ANKRD16 co-localized with cytoplasmic, but not nuclear, aggregates of GFP-GCP170. Similarly, only weak association of ANKRD16 was seen with nuclear aggregates of the expanded polyglutamine-SCA1-GFP fusion protein (GFP-84Q) in co-transfected cells (data not shown). Combined, these results suggest ANKRD16 interacts with misfolded proteins directly or indirectly, via interactions with other proteins and an interaction with cytoplasmic inclusions may be favored.

For immunostaining the cells were fixed with 4% paraformaldehyde (PFA) at room temperature for 5 min, then incubated with anti-ubiquitin, anti-HA, anti-r-tubulin, and anti-vimentin antibodies overnight at 4° C. For visualization they were then incubated with Alexa488 or Alexa555-conjugate anti-(rabbit IgG) or anti-(mouse IgG) (Invitrogen, Molecular Probes) for 45 min at room temperature, and cell nuclei were labeled with Hoechst 33342 (Sigma-Aldrich). The fluorescence was detected under a fluorescence microscope.

### Example 8

### ANKRD16 Can Modulate the Ubiquitin-Proteasome System in vitro

Full length ANKRD16³⁶¹ can modulate the ubiquitin-proteasome system in sti/sti Purkinje cells and fibroblasts. To test this, we will examine the influence of full-length and the different forms of ANKRD16 predominantly found in non-rescuing strains, on fluorescently tagged, constitutively degraded proteasome substrates that serve as ubiquitin-proteasome system (UPS) reporters. Short-lived ubiquitin-GFP fusion proteins have proven extremely useful in probing the functionality of this degradation pathway both in vitro and in vivo (Bowman et al., 2005a; Heessen et al., 2003; Heessen et al., 2005; Menendez-Benito et al., 2005; Salomons et al., 2005; Verhoef et al., 2002). These fluorescent reporter proteins allow simultaneous probing of multiple aspects of the ubiquitin/proteasome system, rather than individually evaluating the functionality of each of the various steps in this pathway. These substrates have very low background florescence, but are readily accumulated up to 1,000 fold upon the addition of proteasome inhibitors. Because they behave as authentic constitutive protein substrates of the proteasome, these fusion proteins make very reliable tests of the function of this system. Lastly, degradation of most reporter substrates, with the exception of ornithine decarboxylase (ODC)—based substrates, occurs in a ubiquitin-dependent manner (Neefjes and Dantuma, 2004a, b).

To evaluate if ANKRD16 can in fact modulate UPS-mediated degradation, full-length ANKRD16^{CAST}-DSRed or empty vector (control) will be transiently transfected into HEK293 cells stably expressing a GFP^{μ}, a ubiquitin-dependent reporter consisting of a 16 amino acid CL1 degron (which was initially found in yeast to degrade β-galactosidase) fused to the C-terminus of GFP (Bence et al., 2001; Gilon et al., 1998). This cell line was generated by Dr. Ron Kopito and is available through ATCC (CRL-2794). 24 hours post-transfection, proteasome inhibitors will be added and GFP^{μ} levels will be analyzed. Because this cell line has a tight distribution of low GFP intensities, increases in the level of GFP^{μ} expression should be readily quantifiable via flow cytometry. However, since proteosome inhibitors induce GFP^{μ} expression in both the cytoplasm and nucleus, transfected cells will be first observed via epifluorescence microscopy. This will allow us to determine changes in GFP^{μ} expression occur in both the nucleus and cytoplasm in ANKRD16-transfected cells relative to those transfected with control plasmid. If decreases in GFP^{μ} are observed in both cellular compartments, we will quantitate GFF^{μ} transfection in live cells via FACS analysis as described below.

FACS calibrations will be done with non-transfected HEK cells and untreated GFPμ HEK cells transfected with either control DSRed vector or full-length ANKRD16-DSRed. To evaluate the effect of ANKRD16³⁶¹ on GFPμ degradation, transfected cells will be treated with MG-132 and cells will be collected 2, 6, and 10 hours post-treatment. Live cells will be gated on and GFP levels will be analyzed and compared to levels of ANKRD16 (in the red channel) in control and full-length ANKRD16-transfected cells. This strategy will allow correlations with the amount of ANKRD16 expression and GFP expression, which is particularly important if only a subset of transfected cells express sufficient amounts of ANKRD16 to modulate proteasomal degradation of GFPμ. A decrease in the mean GFP fluorescence intensity of ANKRD16-transfected, proteasome-treated cells vs. empty vector-transfected, proteasome-treated cells will indicate a functional role for ANKRD16 in enhancing general UPS function.

Although full-length ANKRD16 is expressed in both the cytoplasm and nucleus in transfected cells, ANKRD16 seems to preferentially accumulate in cytoplasmic aggregates suggestion that it promotes degradation of cytoplasmic GFPμ. GFPμ can be quantitated in transfected cells using fluorescence microscopy. After confirming that the field is uniformly illuminated and that the camera is linear, images will be captured by a CCD camera. Image quantification will provide quantitative data as previously described for GFP^{μ} (Bence et al., 2001). Briefly, cells will be trypsinized to remove them from the dish and lightly fixed in 4% PFA. This procedure promotes rounding so that cells can be easily imaged at the equatorial plan with minimal differences in cell size due to differential spreading. After replating in anti-fade mounting media, ANKRD16-expressing cells (identified by the red channel) will be imaged in both red and green channels using an exposure time that results in image intensities within the linear range of the camera and the highest grayscale bit rate. An integrated pixel intensity of different cellular regions (minus background) will be compiled from 400-500 cells so that mean intensity and standard deviations can be compared between control-transfected and ANKRD16-transfected cells.

If full-length ANKRD16 is found to increase the degradation of either (or both) of these proteasome substrates, we will repeat experiments with constructs encoding DSRed-tagged ANKRD16 forms found in non-rescuing mice. Substrate degradation efficiency will be compared (as described above) between constructs encoding full-length ANKRD16^{CAST}, ANKRD16¹⁻¹⁶⁶(a predominant form in non-rescuing strains and a minor form in CAST mice), ANKRD16 ³⁰, and ANKRD16¹⁻³¹⁹ (also major forms in non-rescuing strains, and full-length ANKRD16^{B6} (the minor B6 form with 2 C-terminal amino acid polymorphisms).

### Example 9

### ANKRD16 Can Modulate the Ubiquitin Proteasome System (UPS) in vivo

Protein aggregates have been shown to interfere with proteasome activity. We hypothesize that misfolded protein accumulation in sti/sti Purkinje cells and serine-treated fibroblasts results in a loss of proteasome activity. Furthermore, we hypothesize that the presence of full-length ANKRD16 acts to enhance UPS function by reducing the misfolded protein load in these cells. To test these hypotheses, we monitored short-lived ubiquitin-GFP fusion proteins in vivo.

Two lines of transgenic mice, UB^{G76V}-1 and UB^{G76V}-2, were generated with a ubiquitin-fusion GFP driven by the chicken β-actin promoter (Lindsten et al., 2003a; Lindsten et al., 2003b). UbG76V-GFP transgenic mice are available from The Jackson Laboratory (stock number 008111 and 008112). These mice ubiquitously express transcripts for the transgene, but as expected from the short half-life of the modified GFP protein, GFP is undetectable in all tissues analyzed. However, the administration of proteasome inhibitors to mice, or to primary cells generated from these mice, causes an elevation of GFP levels in a dose-dependent manner (excluding the brain because of the impermeability of the blood/brain barrier to these compounds). These studies validate the UB^{G76V} mice as a tool to assay constitutive degradation of proteins by the ubiquitin/proteasome pathway. Interestingly, the GFP levels in Purkinje cells of untreated mice from the UbG76V-GFP1 line are relatively high compared to other neurons. This elevation in Purkinje cell GFP expression is not seen in UbG76V-GFP2 mice. However, because of this difference in basal levels of GFP in Purkinje cells between the two transgenic lines, the effect of the sti mutation and the ANKRD16^{CAST} transgene was tested on the UPS in both lines.

Purkinje cells are a relatively small percentage of cells in the cerebellum. Therefore, Western analysis may not detect an increase in GFP levels in sti/sti; UbG67V mice, particularly if this increase is confined to the Purkinje cells, as we predict. Therefore we will also analyze native GFP levels in the cerebellum from mice of the same age and genotypes. This analysis will also allow us to confirm that any observed upregulation of GFP expression is indeed localized to Purkinje cells. Mice will be perfused with 4% PFA and after a brief post fixation, brains will be cryoprotected by immersion in a graded sucrose series and embedded for cryostat sectioning. Analysis will be performed as previously described (Bowman et al., 2005b; Lindsten et al., 2003b). Sagittal sections taken from near midline will be counterstained with Toto-3 (Molecular Probes) and examined by fluorescence microscopy for quantitative analysis of GFP fluorescence levels. To protect against bleaching of GFP and to reduce intersample variability, specimens will be collected concurrently, and kept in the dark prior to imaging on a Leica Sp2 confocal microscope. Z-stacks will be collected at a constant step interval throughout the entire section, and quantitative analysis of GFP will be done using the sum of images throughout the stack.

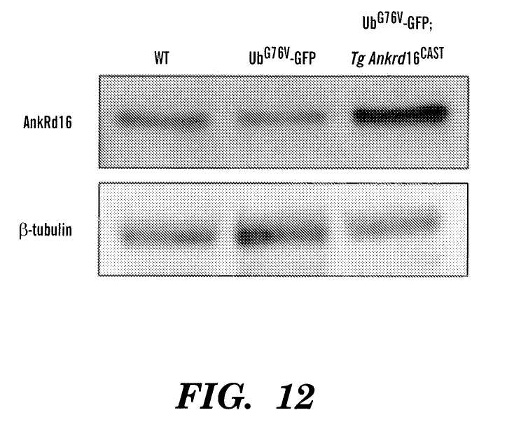

UbG76V-GFP transgenic mice were intercrossed with the BAC transgenic mice carrying the CAST allele of ANKRD16, which gives rise to full length ANKRD16 mRNA and protein, yielding in double transgenic called UbG76V-GFP; ANKRD16^{CAST}. Embryonic fibroblasts were isolated as described earlier from the C57BL/6J wild type (WT), UbG76V-GFP transgenic and UbG76V-GFP; ANKRD16^{CAST} double transgenic mice. Using Western blot analysis the expression of ANKRD16 was analyzed showing an upregulation of ANKRD16 in the double transgenic mice UbG76V-GFP; ANKRD16 6^{CAST}. Beta-tubulin was used for normalization (FIG. 12).

Differences in GFP expression were observed between genotypes by immunofluorescence (data not shown). Quantitative confocal microscopy can be performed to analyze GFP levels in Purkinje cells that have bright punctate ubiquitin staining versus those that show the normal, diffuse pattern of staining.

Proteasome function in serine-induced cell death in sti/sti fibroblasts. To determine if ANKRD16 modulates proteasome function during serine-induced cell death in mouse embryonic fibroblasts, fibroblasts were prepared from E13.5 F2 embryos that carry the UbG76V-GFP transgene, the Ankrd16^{CAST}, both transgenes and +/+ (controls).

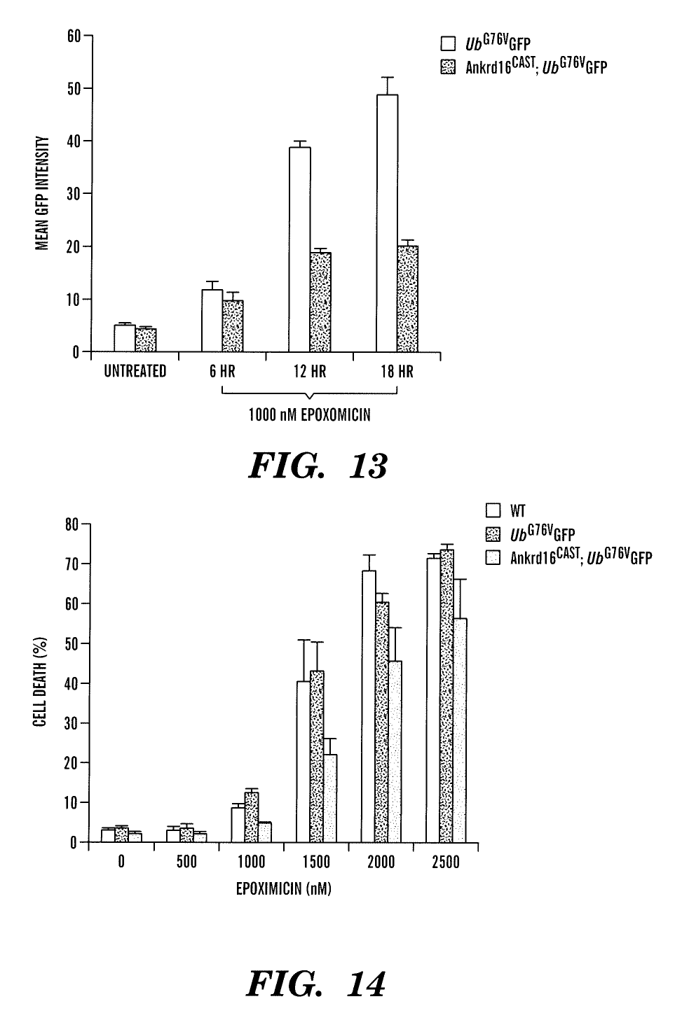

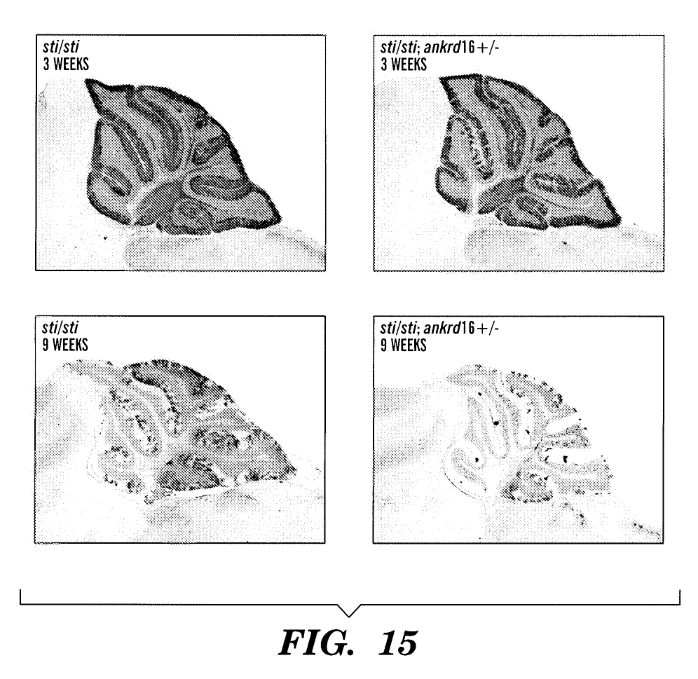

Mouse embryonic fibroblasts were treated with 1.5 uM epoximicin for 6 hours before the GFP (green fluorescent protein) levels were analyzed by fluorescence microscopy (data not shown) or fluorescence-activated cell sorting (FACS) analysis (FIG. 13). Mouse embryonic fibroblasts not expressing the ANKRD16^{CAST} allele show a higher GFP level. In the fibroblasts derived from the UbG76V-GFP; ANKRD16^{CAST} double transgenic mice, the green fluorescence from the GFP activity is greatly reduced. The proteasome inhibitor epoximicin prevents the rapid degradation of GFP in the proteasome. This data suggests that ANKRD16^{CAST} expression upregulates proteasome degradation of UBG76V. Further the embryonic fibroblasts from the C57BL/6J wild type (WT), UbG76V-GFP transgenic and UbG76V-GFP; ANKRD16^{CAST} double transgenic mice were treated with various concentrations of epoximicin for 18 hours. The epoximicin concentrations ranged from 400 nM to 2500 nM (see FIG. 14). Fibroblasts were stained with propidium iodide to evaluate the percentage of cell death by FACS analysis. Cells expressing the ANKRD16^{CAST} allele show reduced cell death.

To prevent possible dilution of misfolded proteins upon cell division, fibroblasts will be treated with mitomycin C for 2 hours. 24 hours later, cells will be treated with increasing doses of serine, which increases misfolded proteins in sti/sti fibroblasts. Cells will be lyzed in protein extraction buffer after 24 hours of treatment, and Western analysis will be performed using antibodies to GFP. To correlate levels of misfolded proteins and GFP, blots will be stripped and reprobed with antibodies to ubiquitin. To further quantitate GFP fluorescence, cells will be analyzed by flow cytometry to determine mean fluorescence of each sample.

ANKRD16 co-localizes with ubiquitinated misfolded proteins in transfected cells, suggesting it may directly bind these proteins. We hypothesize this interaction occurs via binding of the putative UBA domain on the C-terminus to polyubiquitin chains on proteins targeted to the proteosome. The predominant forms of ANKRD16 in B6 and other non-rescuing strains are truncated at the C-terminus and do not contain the UBA domain (see FIG. 3). To determine if ANKRD16 may function in part via this putative UBA domain, epitope-tagged full-length ANKRD16^{CAST}, ANKRD16B6 (the minor B6 form with two C-terminal amino acid polymorphisms) and the three truncated forms in will be used in aggresome association experiments, ubiquitin binding assays and polyubiquitinated protein pulldown assays.

To determine if the C-terminus of ANKRD16 containing the UBA domain is necessary for aggresome association, we will transfect COS-7 and A549 cells with various forms of FLAG-tagged ANKRD16 and GFP-CTFR-ΔF508 (which is polyubiquitinated and degraded by the proteasome, but in the absence of proteosome inhibitors accumulates into a single aggresome (Johnston et al., 1998)). 24 hours post-transfection, MG132 will be added. Cells will be immunostained with FLAG antibodies at 6, 12, and 24 hours post-treatment to look for co-localization of truncated ANKRD16 with the GFP-CTFR-ΔF508 aggresome. Co-immunoprecipitations of the FLAG-tagged forms of ANKRD16 with CTFR-ΔF508 will be performed in the presence or absence of proteosome inhibitors using equal amounts of input protein. Westerns will be blotted with antibodies to GFP analyze amount of immunoprecipitated protein. Blots will be striped and reprobed with ubiquitin to confirm CTFR-ΔF508 was polyubiquitinated, as previous described (Kawaguchi et al., 2003), in the presence of MG132.

To determine if ANKRD16 localizes to non-polyubiquitinated aggresomes, we will co-tranfect cells with full-length FLAG-tagged ANKRD16 constructs and GFP-250 (obtained from Dr. Elizabeth Sztul), which when over-expressed spontaneously forms polyubiquitin-deficient aggresomes (Garcia-Mata et al., 1999b). Cells will be immunostained with FLAG antibodies to determine if ANKRD16 is also recruited to the aggresome in the absence of poly-ubiquitination of aggresome proteins. Results will be confirmed by co-IP experiments as described above.

UBA domains have been found to interact with both monoubiquitin and polyubiquitin chains, but usually with higher affinity to polyubiquitin (Buchberger, 2002). To test for interaction of ANKRD16 with ubiquitin, we perform GST-ubiquitin precipitation assays as previously described (Raasi et al., 2005; Wilkinson et al., 2001). COST cells will be transiently transfected with FLAG-tagged full-length ANKRD16^{CAST}, full-length ANKRD16^{B6}, and the truncated ANKRD16 forms. For control of transfection efficiency, all constructs will be co-transfected with an EGFP expressing vector. Cytostolic extracts will be prepared from transfected cells and incubated with glutathione-separose beads alone, or beads conjugated to monoubiquitin, tetraubiquitin, or K48 or K63-linked polyubiquitin chains (Affiniti Research Products). After incubation, protein-GSH sepharose will be pelleted, washed, and bound proteins eluted. The amount of ANKRD16 bound to multi-ubiquitin will be evaluated by western blot with FLAG antibodies. As a control, western analysis of input extract with FLAG and GFP antibodies will also be performed to confirm that equal amounts of input protein were used in pull-down assays. To confirm the specificity of ubiquitin interactions, an excess of free ubiquitin will be added to the extract during incubation to compete with GST-bound ubiquitin for interaction with ANKRD16.

To confirm results obtained from Ub pull-down assays, we will perform COST cell transfections with the ANKRD16 vectors described above in the presence and absence of the proteasome inhibitor MG-123. Cell lysates will be immunoprecipitated with anti-FLAG antibodies and eluted proteins analyzed by western analysis with anti-ubiquitin antibodies. Transfection efficiency and the amount of input protein will be analyzed as described above.

### Example 10

### ANKRD16 Antibody Production

cDNA corresponding to the N-terminal 154 amino acids of ANKRD16 was amplified by PCR and cloned into the bacterial GST fusion expression vector pGEX-4T (GE Healthcare Life Sciences). Purified protein was used to immunize two rabbits and each rabbit was boosted 4 times. Antisera were purified over a GST column to remove GST-specific antibodies. Both antisera recognized ANKRD16 by immunocytochemistry and Western blot analysis in overexpression transfection assays. The antisera reactivity was tested by immunohistochemical and Western blot analysis of C57BL/6, CAST, sti/sti, and Stim (ANKRD16), sti/sti brain extracts (see FIG. 10 for Western blot for C57BL/6 and CAST). Protein samples were run on a 10% SDS-PAGE and transferred onto a nitrocellulose filter. After blocking with 5% non-fat dry milk powder, the membranes were processed through sequential incubations with primary antibody followed by secondary antibody. Immunoreactive proteins on the filter were visualized using a chemiluminescent detection kit (SuperSignal West PICO, Pierce, USA). Antibody specificity was confirmed by analysis of ANKRD16^{-/-} tissues (FIG. 10).

### Example 11

### Expression of ANKRD16 Restored the Reduced Fertility in Sticky Mutant Mice

Female mice of the various genotypes at the age of at 8-9 weeks were mated and the fertility was determined by counting the number of live offspring. The results are shown in FIG. 7. When mating C57BL/6J mice (WT), which are wild type, the average litter size was 6.87 from 8 matings with a total of 55 offspring. Mice homozygous for the sticky (sti) mutation had a litter size of 4.14 from 8 matings with a total of 29 offspring. When the modifier gene ANKRD16^{CAST} was crossed into the sti/sti mutations, the litter size was restored to normal with 6.51 pups from 7 matings with a total of 46 offspring. This demonstrates that the expression of full length ANKRD16 can restore the reduced fertility in sticky mutant mice. The CAST ANKRD16 is the full length version of ANKRD16, also called ANKRD³⁶¹.

### Example 12

### Recombinant Protein Expression of ANKRD16 Protein in Escherichia coli

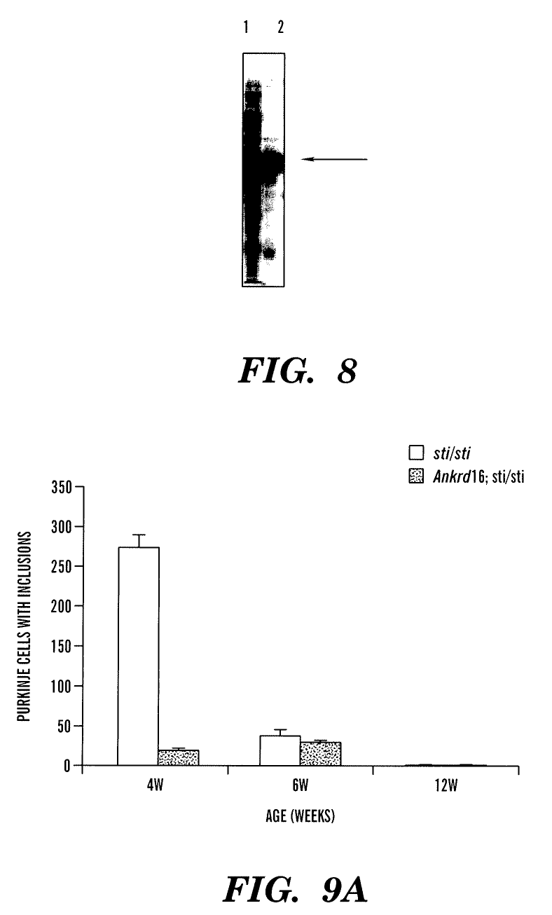

The full length cDNA for ANKRD16 was cloned into the pGEX4T2 vector (GE Healthcare) using standard molecular biology techniques. The Escherichia coli strain BL21(D3) was transformed with Ankrd16-pGEX4T2 expression plasmid and cultured in 4 ml of LB (Luria-Bertani) media containing ampicillin. The following day 2 ml culture of the transformed Escherichia coli culture was transferred into 40 ml of LB media containing ampicillin. After 3 hours incubation at 30 degree Celsius, Ankrd16-GST fusion protein expression was induced by adding IPTG (isopropyl-D-thiogalactopyranoside) with the final concentration of 0.5 mM for 4 hours at 30 degree Celsius. The Escherichia coli cells were harvested by centrifugation. The Escherichia coli pellet from 10 ml culture was dissolved by sonication in 1 ml of lysis buffer (20 mM Tris, 140 mM NaCl, 1% Triton X100). The supernatant of the Escherichia coli lysate was combined with 20 ul volume of glutathione sepharose 4B (GE Healthcare) for purication. For binding of the recombinant protein to the sepharose, the mixture was incubated for 2 hours at 4 degree Celsius. The sepharose was washed for 5 times with washing buffer (20 mM Tris, 140 mM NaCl, 0.1% Triton X100 according to the manufacturer's instructions (GE Healthcare). FIG. 8 shows the Escherichia coli lysate before purification (lane 1) and after one purification round with glutathione sepharose 4B purification (lane 2). The arrow indicates the tagged ANKRD16 recombinant protein.

### Example 13

### ANKRD16 Reduces Protein Inclusions in sti/sti Purkinje Cells

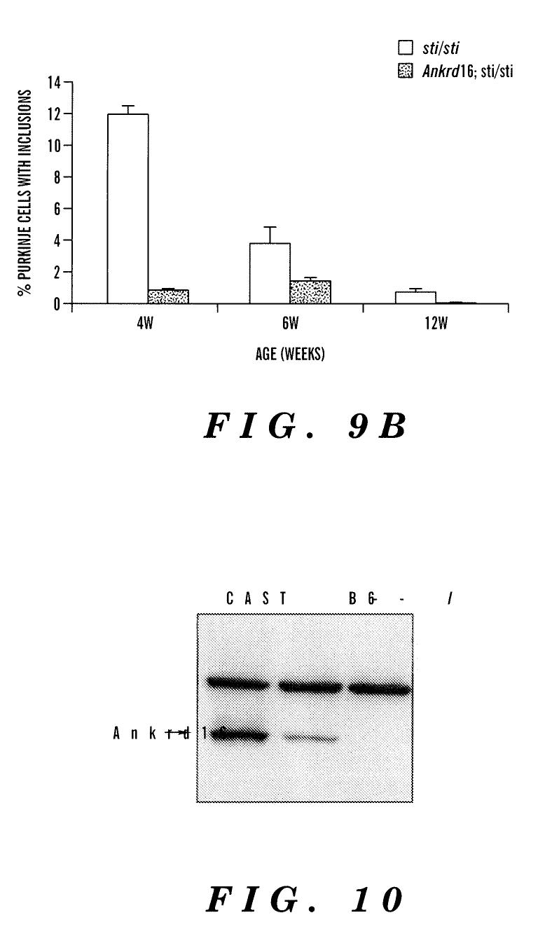

Brains from homozygous sticky mice (sti/sti; B6.Cg-Aarssti/J; available from The Jackson Laboratory Stock Number 002560) and sticky mice intercrossed with transgenic mice expressing the full length ANKRD16, were isolated at the age of 4 weeks, 6 weeks and 12 weeks. The brains were processed for sectioning and antibody staining using standard histological methods. Sagittal sections were prepared and immunostained with antibodies against ubiquitin and calbindin. The antibodies against calbidin D-28 were used to visualize Purkinje cells. The antibodies against ubiquitin were used to visualize ubiquitinated protein aggregates. Purkinje cells with ubiquitinated aggregates were counted from three sections spaced at 100 micrometer beginning at midline and working laterally per mouse. Three mice from each genotype were analyzed. The total number of Purkinje cells with inclusions as well as the % of remaining Purkinje cells with inclusions are shown in the graphs in FIGS. 9A and 9B. At all time points analyzed, the overexpression of full length ANKRD16 reduces Purkinje cells with protein aggregates, being most dramatic at the age of four weeks.

### Example 14

### ANKRD16 Protein is Expressed at Higher Levels in the Cerebellum of CAST/EiJ Mice

To analyze if there is a correlation between mRNA and protein expression, we prepared extracts from the cerebellum of CAST/EiJ and C57BL/6J (B6) and ANKRD16 deficient (−/−) mice. ANKRD16 deficient mice were generated using standard gene targeting methods with homologous recombination in embryonic stem (ES) cells resulting in mouse lacking the ANKRD16 gene (see Example 17). The cerebellums from these mouse strains were isolated and the protein extract was electrophoresed and Western blot analysis was performed. The polyclonal antibody against ANKRD16 was produced by immunizing rabbits with ANKRD16 protein (see Example 10). The antibody used here cross-reacts with another higher molecular protein in the cerebellum. This cross reaction has not been observed in fibroblasts. FIG. 10 shows that the highest amount of ANKRD16 protein can be detected in CAST/EiJ and a lower amount in the C57B1/6J (B6) mice. As expected, the knockout mice (−/−) do not express the ANKRD16 protein.

### Example 15

### Proteasomal Stress Induces Aggresome Localization of ANKRD16

Mouse embryonic fibroblasts were isolated from CASTEi/J mice. To isolate embryonic fibroblasts, CASTEi/J mice were paired for time of pregnancy. E13.5 day pregnant female mice for each of the strains were sacrificed. The time point E0.5 is the morning of finding a plug after pairing a female with a male. The uteri were removed and placed into a dish with PBS and washed. The embryos were isolated free of extraembryonic tissue and washed in PBS. Each embryo was placed into a separate 14 ml tube containing 3 ml of media (DMEM, 10% FBS) and homogenized for 2 seconds. The homogenized tissues were put into 145 mm tissue culture plates (e.g. CellStar from USA Scientific cat.#5663-9160) containing 20 ml of media (DMEM, 10% FBS). The cells were incubated at 37° C. in 5% CO2 until cells were confluent, which was on average from 5 to 7 days. The cells were expanded according to standard tissue culture methods using trypsin and the same passage number for each cell line was used for the experiment. To stress the fibroblast they were treated for 18 hours with 1 μM epoximicin, a proteosome inhibitor (Genaxxon). Then untreated and treated cells were fixed, visualized for ANKRD16 expression using indirect immunofluorescence with polyclonal ANKRD16 rabbit antibody as primary antibody and Alexa555-conjugated anti-rabbit IgG (Invitrogen, Molecular Probes) as secondary antibody. For counterstaining DAPI was used which stains the nuclei blue. In untreated fibroblasts, ANKRD16 is normally distributed throughout the cytoplasm with some puncta in the nucleus. In epoximicin-treated fibroblasts ANKRD16 is relocalized to the aggresome (data not shown).

### Example 16

### Localization of ANKRD16 to Cytoplasmic Aggregates

Cells were cotransfected with Hemagglutinin epitope (HA)-tagged ANKRD16 and plasmids encoding proteins that spontaneously misfold. The HA-epitope was inserted at the C-terminus of full length ANKRD16. As example for a protein that misfolds spontaneously GFP170 was used. GFP170 is a chimeric protein produced from the fusion of green fluorescent protein (GFP) to a segment amino acids 566-1375) of the Golgi Complex Protein (GCP170) (for reference see Fu et al., Molecular Biol. of the Cell (2005) 16:4905-4917).

The cotransfection of HA epitope tagged Ankrd16 with GFP170 leads to colocalization of ANKRD16 with these misfolded proteins in the cytoplasm (data not shown). GFP 170 spontaneously forms both nuclear and cytoplasmic aggregates, and ANKRD16 co-localizes with the cytoplasmic, but not nuclear aggregates (data not shown). ANKRD16 was visualized by indirect immunofluorescence using a primary polyclonal rabbit antibody and a secondary Alexa555-conjugated anti-rabbit IgG (Invitrogen, Molecular Probes), which gives a red signal.

### Example 17

### Generation of a Conditional ANKRD16 Allele and Null ANKRD16 Allele in Mice

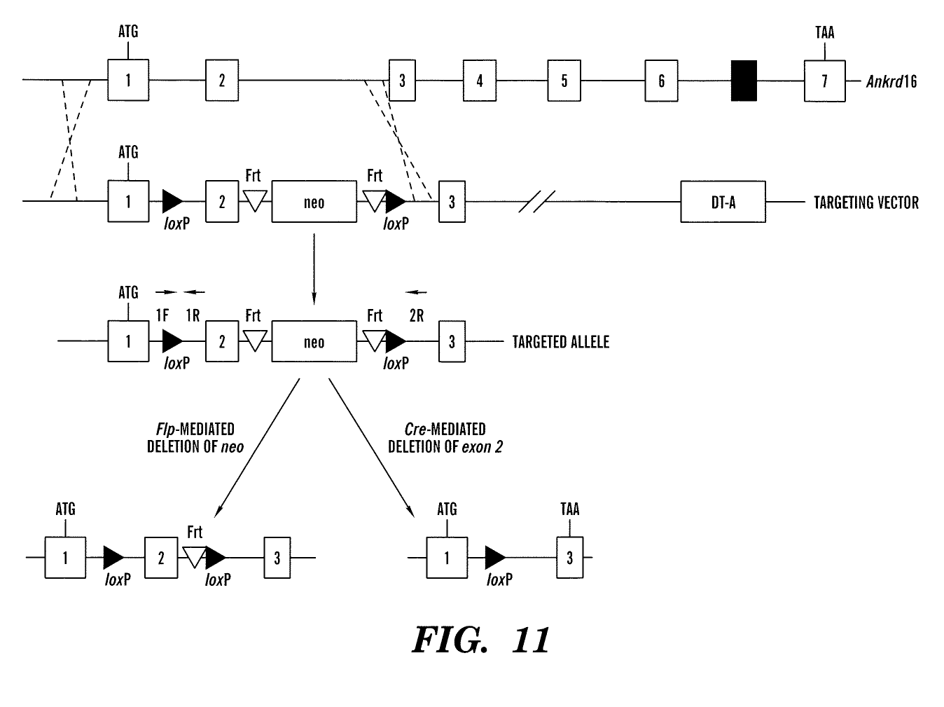

The mouse ANKRD16 gene is approximately 10 kb with 7 coding exons and an additional alternatively spliced exon (exon 6′) in C57BL/6. To generate a loss of function mutation, we targeted exon 2 that is utilized in all transcripts. Deletion of this exon leads to a frame shift and stop codon in the non-alternatively spliced exon 3. Thus at best, a truncated 104 amino acid peptide will be produced. To construct the targeted allele, we used two independent site-specific recombinase systems for excision of the neo-positive selection cassette and conditional gene inactivation in the animal. The one site-specific recombinase system used is known as the Cre-Lox recombination with the loxP site (34 bp) providing the site-specific sites for the Cre recombinase. One loxP site was introduced into intron 1 of the ANKRD16 gene. The second loxP site was engineered on the 3′ end of the second Frt site in intron 2. The other site-specific recombinase system used is the FLP-FRT recombination with Frt (Flipase Recognition Target) sites providing the site-specific sites for the Flp recombinase. The neomycin (neo) selection cassette was flanked by Frt sites in intron 2. This construction scheme allows us to remove the neo cassette in vivo with Flp without the problems associated with manipulation of ES cells with three Cre sites. Cre-mediated excision will not only remove exon 2, but also the neo-selection cassette, leading to a null mutation (See FIG. 11). Further, the intact FRT/neo cassette may produce a new hypomorphic allele that could be very useful for additional testing of partial loss of gene function.

Using this strategy, R1 ES cells were electroporated and selected for neomycin resistance. Clones were screened by PCR and Southern analysis to identify correctly targeted events. Three targeted clones were injected into C57BL/6J (B6) blastocysts, and transplanted into pseudopregnant recipient mice. Several male founder animals with extensive coat color chimerism were mated to C57BL/6J females, and agouti pups were genotyped for the targeted event. Germline transmission of the targeted allele was obtained for all three clones. Mice heterozygous for the floxed allele were mated to a ubiquitous Cre-deleter mouse strain, EIIA-Cre (B6.FVB-Tg(EIIa-cre)C5379Lmgd/J available from The Jackson Laboratory stock number 003724), and proper deletion of exon 2 was detected by PCR analysis (data not shown). PCR data showed the correct deletion in the presence of Cre recombinase. The PCR primers used are flanking either the distal loxP site or the exon2/neo cassette and are indicated in panel A as 1F, 1R and 2R, with F standing for forward primer and R for reverse primer.

These data show that ANKRD16 null mice can be generated by intercrossing the heterozygous mice carrying the exon 2 deletion.

### Example 18

### Analysis of ANKRD16 Gene Dosage Effects in the Cerebellum

The sticky (sti) mutation homozygous on C57BL/6J (B6) background (B6.stock-sti/sti; B6.Cg-Aarssti/J; available from The Jackson Laboratory Stock Number 002560) was intercrossed with ANKRD16 knockout mice, which are described in Example 17. The ANKRD16 knockout mice are on a (129×1/SvJ and 129S1/SV-+p+Tyr-cKitlS1-J/+)F1 and C57BL/6J mixed background and do not carry the CAST allele for ANKRD16. The brain from homozygous sticky mice (sti/sti) and mice homozygous for sticky, but heterozygous for ANKRD16 (sti/sti; ANKRD16+/−) was isolated from 3 week and 9 week old mice, fixed and processed for sagittal sections. The sections were immunostained with antibodies to calbindin D-28, which is highly expressed in Purkinje cells (FIG. 14). The results are as follows: the absence of calbindin D-28 expression correlated with Purkinje cell death. There were less neurons in the cerebellum of mice lacking one copy of ANKRD16, thus having a lower expression of ANKRD16. The abundance of calbindin D-28 expression at the age of 3 weeks, with sti/sti; ANKRD16+/− mice showed a reduced level of calbindin D-28. At the age of 9 weeks a dramatic difference in the expression of calbindin D-28 was observed, compared sti/sti to the sti/sti; ANKRD16+/− with a very low level of calbindin D-28 expression in sti/sti; ANKRD16+/− mice. This suggests that ANKRD16 modulates Purkinje cell survival in a gene dosage-dependent manner; i.e. high levels of full length ANKRD16 is protective and disease progression correlates with lower levels of ANKRD16 expression.

### INCORPORATION BY REFERENCE

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

### REFERENCES

Arrasate, M., Mitra, S., Schweitzer, E. S., Segal, M. R., and Finkbeiner, S. (2004). Inclusion body formation reduces levels of mutant huntingtin and the risk of neuronal death. Nature 431, 805-810.
Auluck, P. K., Chan, H. Y. E., Trojanowski, J. Q., Lee, V. M. Y., and Bonini, N. M. (2002). Chaperone suppression of alpha-synuclein toxicity in a Drosophila model for Parkinson's disease. Science 295, 865-868.
Barral, J. M., Broadley, S. A., Schaffar, G., and Hartl, F. U. (2004). Roles of molecular chaperones in protein misfolding diseases. Seminars in Cell and Developmental Biology 15, 17-29.
Bence, N. F., Sampat, R. M., and Kopito, R. R. (2001). Impairment of the ubiquitin-proteasome system by protein aggregation. Science 292, 1552-1555.
Berger, Z., Ravikumar, B., Menzies, F. M., Oroz, L. G., Underwood, B. R., Pangalos, M. N., Schmitt, I., Wullner, U., Evert, B. O., O'Kane, C. J., et al. (2006). Rapamycin alleviates toxicity of different aggregate-prone proteins. Human Molecular Genetics 15, 433-442.
Bossy-Wetzel, E., Schwarzenbacher, R., and Lipton, S. A. (2004). Molecular pathways to neurodegeneration. Nature Medicine 10.
Bowman, A. B., Yoo, S. Y., Dantuma, N. P., and Zoghbi, H. Y. (2005a). Neuronal dysfunction in a polyglutamine disease model occurs in the absence of ubiquitin-proteasome system impairment and inversely correlates with the degree of nuclear inclusion formation. Human Molecular Genetics 14, 679-691.
Bowman, A. B., Yoo, S. Y., Dantuma, N. P., and Zoghbi, H. Y. (2005b). Neuronal dysfunction in a polyglutamine disease model occurs in the absence of ubiquitin-proteasome system impairment and inversely correlates with the degree of nuclear inclusion formation. Hum Mol Genet 14, 679-691.
Buchberger, A. (2002). From UBA to UBX: new words in the ubiquitin vocabulary. Trends Cell Biol 12, 216-221.
Caughey, B., and Lansbury Jr, P. T. (2003). Protofibrils, pores, fibrils, and neurodegeneration: Separating the responsible protein aggregates from the innocent bystanders. Annual Review of Neuroscience 26, 267-298.
Chen, H. Y. E., Warrick, J. M., Andriola, I., Merry, D., and Bonini, N. M. (2002). Genetic modulation of polyglutamine toxicity by protein conjugation pathways in Drosophila. Human Molecular Genetics 11, 2895-2904.
Chung, K. K., Dawson, V. L., and Dawson, T. M. (2001). The role of the ubiquitin-proteasomal pathway in Parkinson's disease and other neurodegenerative disorders. Trends in Neurosciences 24.
Cohen, F. E., and Kelly, J. W. (2003). Therapeutic approaches to protein-misfolding diseases. Nature 426, 905-909.
Cuervo, A. M. (2004). Autophagy: In sickness and in health. Trends in Cell Biology 14, 70-77.
Cummings, C. J., Sun, Y., Opal, P., Antalffy, B., Mestril, R., Orr, H. T., Dillman, W. H., and Zoghbi, H. Y. (2001). Over-expression of inducible HSP70 chaperone suppresses neuropathology and improves motor function in SCA1 mice. Human Molecular Genetics 10, 1511-1518.
Dawson, S., Apcher, S., Mee, M., Higashitsuji, H., Baker, R., Uhle, S., Dubiel, W., Fujita, J., and Mayer, R. J. (2002). Gankyrin is an ankyrin-repeat oncoprotein that interacts with CDK4 kinase and the S6 ATPase of the 26 S proteasome. Journal of Biological Chemistry 277, 10893-10902.
Dawson, S., Higashitsuji, H., Wilkinson, A. J., Fujita, J., and Mayer, R. J. (2006). Gankyrin: a new oncoprotein and regulator of pRb and p53. Trends in Cell Biology 16, 229-233.
Dawson, T. M., and Dawson, V. L. (2003). Molecular Pathways of Neurodegeneration in Parkinson's Disease. Science 302, 819-822.
Deveraux, Q., Ustrell, V., Pickart, C., and Rechsteiner, M. (1994). A 26 S protease subunit that binds ubiquitin conjugates. Journal of Biological Chemistry 269, 7059-7061.
Elsasser, S., and Finley, D. (2005). Delivery of ubiquitinated substrates to protein-unfolding machines. Nature Cell Biology 7, 742-749.
Fink, A. L. (1999). Chaperone-mediated protein folding. Physiological Reviews 79, 425-449.
Fu, L., Gao, Y. S., Tousson, A., Shah, A., Chen, T. L. L., Vertel, B. M., and Sztul, E. (2005). Nuclear aggresomes form by fusion of PML-associated aggregates. Molecular Biology of the Cell 16, 4905-4917.
Gandhi, S., and Wood, N. W. (2005). Molecular pathogenesis of Parkinson's disease. Human Molecular Genetics 14, 2749-2755.
Garcia-Mata, R., Bebok, Z., Sorscher, E. J., and Sztul, E. S. (1999a). Characterization and dynamics of aggresome formation by a cytosolic GFP-chimera. Journal of Cell Biology 146, 1239-1254.
Garcia-Mata, R., Bebok, Z., Sorscher, E. J., and Sztul, E. S. (1999b). Characterization and dynamics of aggresome formation by a cytosolic GFP-chimera. J Cell Biol 146, 1239-1254.
Gatchel, J. R., and Zoghbi, H. Y. (2005). Diseases of unstable repeat expansion: mechanisms and common principles. Nature Reviews Genetics 6, 743-755.
Giffard, R. G., Xu, L., Zhao, H., Carrico, W., Ouyang, Y., Qiao, Y., Sapolsky, R., Steinberg, G., Hu, B., and Yenari, M. A. (2004). Chaperones, protein aggregation, and brain protection from hypoxic/ischemic injury. Journal of Experimental Biology 207, 3213-3220.
Gilon, T., Chomsky, O., and Kulka, R. G. (1998). Degradation signals for ubiquitin system proteolysis in Saccharomyces cerevisiae. EMBO Journal 17, 2759-2766.
Glickman, M. H. (2000). Getting in and out of the proteasome. Seminars in Cell and Developmental Biology 11, 149-158.
Glickman, M. H., and Ciechanover, A. (2002). The ubiquitin-proteasome proteolytic pathway: Destruction for the sake of construction. Physiological Reviews 82, 373-428.
Glickman, M. H., Rubin, D. M., Fried, V. A., and Finley, D. (1998). The regulatory particle of the Saccharomyces cerevisiae proteasome. Molecular and Cellular Biology 18, 3149-3162.
Gregersen, N., Bolund, L., and Bross, P. (2005). Protein misfolding, aggregation, and degradation in disease. Molecular Biotechnology 31, 141-150.
Gutekunst, C. A., Li, S. H., Yi, H., Mulroy, J. S., Kuemmerle, S., Jones, R., Rye, D., Ferrante, R. J., Hersch, S. M., and Li, X. J. (1999). Nuclear and neuropil aggregates in Huntington's disease: Relationship to neuropathology. Journal of Neuroscience 19, 2522-2534.
Hall, G. F., and Yao, J. (2005). Modeling tauopathy: A range of complementary approaches. Biochimica et Biophysica Acta—Molecular Basis of Disease 1739, 224-239.
Hansen, J. J., Durr, A., Cournu-Rebeix, I., Georgopoulos, C., Ang, D., Nielsen, M. N., Davoine, C. S., Brice, A., Fontaine, B., Gregersen, N., et al. (2002). Hereditary spastic paraplegia SPG13 is associated with a mutation in the gene encoding the mitochondrial chaperonin Hsp60. American Journal of Human Genetics 70, 1328-1332.
Hartl, F. U., and Hayer-Hartl, M. (2002). Protein folding. Molecular chaperones in the cytosol: From nascent chain to folded protein. Science 295, 1852-1858.
Heessen, S., Dantuma, N. P., Tessarz, P., Jenne, M., and Masucci, M. G. (2003). Inhibition of ubiquitin/proteasome-dependent proteolysis in Saccharomyces cerevisiae by a Gly-Ala repeat. FEBS Letters 555, 397-404.
Heessen, S., Masucci, M. G., and Dantuma, N. P. (2005). The UBA2 domain functions as an intrinsic stabilization signal that protects rad23 from proteasomal degradation. Molecular Cell 18, 225-235.
Higashitsuji, H., Higashitsuji, H., Itoh, K., Sakurai, T., Nagao, T., Sumitomo, H., Masuda, T., Dawson, S., Shimada, Y., Mayer, R. J., et al. (2005). The oncoprotein gankyrin binds to MDM2/HDM2, enhancing ubiquitylation and degradation of p53. Cancer Cell 8, 75-87.
Higashitsuji, H., Itoh, K., Nagao, T., Dawson, S., Nonoguchi, K., Kido, T., Mayer, R. J., Arii, S., and Fujita, J. (2000). Reduced stability of retinoblastoma protein by gankyrin, an oncogenic ankyrin-repeat protein overexpressed in hepatomas. Nature Medicine 6, 96-99.
Homma, S., Iwasaki, M., Shelton, G. D., Engvall, E., Reed, J. C., and Takayama, S. (2006). BAG3 deficiency results in fulminant myopathy and early lethality. American Journal of Pathology 169, 761-773.
Hori, T., Kato, S., Saeki, M., DeMartino, G. N., Slaughter, C. A., Takeuchi, J., Toh-e, A., and Tanaka, K. (1998). cDNA cloning and functional analysis of p28 (Nas6p) and p40.5 (Nas7p), two novel regulatory subunits of the 26S proteasome. Gene 216, 113-122.
Johnston, J. A., Ward, C. L., and Kopito, R. R. (1998). Aggresomes: a cellular response to misfolded proteins. J Cell Biol 143, 1883-1898.
Kawaguchi, Y., Kovacs, J. J., McLaurin, A., Vance, J. M., Ito, A., and Yao, T. P. (2003). The deacetylase HDAC6 regulates aggresome formation and cell viability in response to misfolded protein stress. Cell 115, 727-738.
Klucken, J., Shin, Y., Masliah, E., Hyman, B. T., and McLean, P. J. (2004). Hsp70 reduces alpha-synuclein aggregation and toxicity. Journal of Biological Chemistry 279, 25497-25502.
Kopito, R. R., and Ron, D. (2000). Conformational disease. Nature Cell Biology 2. Kopito, R. R., and Sitia, R. (2000). Aggresomes and Russell bodies-Symptoms of cellular indigestion? EMBO Reports 1, 225-231.
Kuemmerle, S., Gutekunst, C. A., Klein, A. M., Li, X. J., Li, S. H., Beal, M. F., Hersch, S. M., and Ferrante, R. J. (1999). Huntingtin aggregates may not predict neuronal death in Huntington's disease. Annals of Neurology 46, 842-849.
Lam, Y. A., Lawson, T. G., Velayutham, M., Zweler, J. L., and Pickart, C. M. (2002). A proteasomal ATPase subunit recognizes the polyubiquitin degradation signal. Nature 416, 763-767.
Lee, J. W., Beebe, K., Nangle, L. A., Jang, J., Longo-Guess, C. M., Cook, S. A., Davisson, M. T., Sundberg, J. P., Schimmel, P., and Ackerman, S. L. (2006). Editing-defective tRNA synthetase causes protein misfolding and neurodegeneration. Nature 443, 50-55.
Leggett, D. S., Hanna, J., Borodovsky, A., Crosas, B., Schmidt, M., Baker, R. T., Walz, T., Ploegh, H., and Finley, D. (2002). Multiple associated proteins regulate proteasome structure and function. Molecular Cell 10, 495-507.
Leroy, E., Boyer, R., Auburger, G., Leube, B., Ulm, G., Mezey, E., Harta, G., Brownstein, M. J., Jonnalagada, S., Chernova, T., et al. (1998). The ubiquitin pathway in Parkinson's disease. Nature 395, 451-452.
Levine, B., and Yuan, J. (2005). Autophagy in cell death: An innocent convict? Journal of Clinical Investigation 115, 2679-2688.
Lindquist, S. (1986). The heat-shock response. Annual Review of Biochemistry VOL. 55, 1151-1191.
Lindsten, K., Menendez-Benito, V., Masucci, M. G., and Dantuma, N. P. (2003a). A transgenic mouse model of the ubiquitin/proteasome system. Nature Biotechnology 21, 897-902.
Lindsten, K., Menendez-Benito, V., Masucci, M. G., and Dantuma, N. P. (2003b). A transgenic mouse model of the ubiquitin/proteasome system. Nat Biotechnol 21, 897-902.
Mata, I. F., Lockhart, P. J., and Farrer, M. J. (2004). Parkin genetics: One model for Parkinson's disease. Human Molecular Genetics 13.
Mayer, M., Reinstein, J., and Buchner, J. (2003). Modulation of the ATPase cycle of BiP by peptides and proteins. Journal of Molecular Biology 330, 137-144.
Mayer, M. P., and Bukau, B. (2005). Hsp70 chaperones: Cellular functions and molecular mechanism. Cellular and Molecular Life Sciences 62, 670-684.
Mayer, R. J., and Fujita, J. (2006). Gankyrin, the 26 S proteasome, the cell cycle and cancer. Biochemical Society Transactions 34, 746-748.
McNaught, K. S. P., Perl, D. P., Brownell, A. L., and Olanow, C. W. (2004). Systemic exposure to proteasome inhibitors causes a progressive model of Parkinson's disease. Annals of Neurology 56, 149-162.
Menendez-Benito, V., Heessen, S., and Dantuma, N. P. (2005). Monitoring of ubiquitin-dependent proteolysis with green fluorescent protein substrates. Methods in Enzymology 399, 490-511.
Muchowski, P. J. (2002). Protein misfolding, amyloid formation, and neurodegeneration: A critical role for molecular chaperones? Neuron 35, 9-12.
Muchowski, P. J., and Wacker, J. L. (2005). Modulation of neurodegeneration by molecular chaperones. Nature Reviews Neuroscience 6, 11-22.
Neefjes, J., and Dantuma, N. P. (2004a). Fluorescent probes for proteolysis: tools for drug discovery. Nat Rev Drug Discov 3, 58-69.
Neefjes, J., and Dantuma, N. P. (2004b). Fluorescent probes for proteolysis: Tools for drug discovery. Nature Reviews Drug Discovery 3, 58-69.
Oberdick, J., Smeyne, R. J., Mann, J. R., Zackson, S., and Morgan, J. I. (1990). A promoter that drives transgene expression in cerebellar Purkinje and retinal bipolar neurons. Science 248, 223-226.
Petrucelli, L., O'Farrell, C., Lockhart, P. J., Baptista, M., Kehoe, K., Vink, L., Choi, P., Wolozin, B., Farrer, M., Hardy, J., et al. (2002). Parkin protects against the toxicity associated with mutant ?-Synuclein: Proteasome dysfunction selectively affects catecholaminergic neurons. Neuron 36, 1007-1019.
Raasi, S., Varadan, R., Fushman, D., and Pickart, C. M. (2005). Diverse polyubiquitin interaction properties of ubiquitin-associated domains. Nat Struct Mol Biol 12, 708-714.
Ravikumar, B., Acevedo-Arozena, A., Imarisio, S., Berger, Z., Vacher, C., O'Kane, C. J., Brown, S. D. M., and Rubinsztein, D. C. (2005). Dynein mutations impair autophagic clearance of aggregate-prone proteins. Nature Genetics 37, 771-776.
Ravikumar, B., Duden, R., and Rubinsztein, D. C. (2002). Aggregate-prone proteins with polyglutamine and polyalanine expansions are degraded by autophagy. Human Molecular Genetics 11, 1107-1117.
Ravikumar, B., Vacher, C., Berger, Z., Davies, J. E., Luo, S., Oroz, L. G., Scaravilli, F., Easton, D. F., Duden, R., O'Kane, C. J., et al. (2004). Inhibition of mTOR induces autophagy and reduces toxicity of polyglutamine expansions in fly and mouse models of Huntington disease. Nature Genetics 36, 585-595.
Reggiori, F. (2006). 1 Membrane Origin for Autophagy. In Current Topics in Developmental Biology, pp. 1-30.
Reggiori, F., and Klionsky, D. J. (2002). Autophagy in the eukaryotic cell. Eukaryotic Cell 1, 11-21.
Robertson, J., Kriz, J., Nguyen, M. D., and Julien, J. P. (2002). Pathways to motor neuron degeneration in transgenic mouse models. Biochimie 84, 1151-1160.
Ross, C. A., and Poirier, M. A. (2004). Protein aggregation and neurodegenerative disease. Nature Medicine 10.
Ross, C. A., and Poirier, M. A. (2005). What is the role of protein aggregation in neurodegeneration? Nature Reviews Molecular Cell Biology 6, 891-898.
Salomons, F. A., Verhoef, L. G. G. C., and Dantuma, N. P. (2005). Fluorescent reporters for the ubiquitin-proteasome system. Essays in Biochemistry 41, 113-128.
Saudou, F., Finkbeiner, S., Devys, D., and Greenberg, M. E. (1998). Huntingtin acts in the nucleus to induce apoptosis but death does not correlate with the formation of intranuclear inclusions. Cell 95, 55-56.
Selkoe, D. J. (2003). Folding proteins in fatal ways. Nature 426, 900-904.
Sherman, M. Y., and Goldberg, A. L. (2001). Cellular defenses against unfolded proteins: A cell biologist thinks about neurodegenerative diseases. Neuron 29, 15-32.
Smeyne, R. J., Chu, T., Lewin, A., Bian, F., Crisman, S. S., Kunsch, C., Lira, S. A., and Oberdick, J. (1995). Local control of granule cell generation by cerebellar Purkinje cells. Molecular and Cellular Neurosciences 6, 230-251.
Soti, C., and Csermely, P. (2000). Molecular chaperones and the aging process. Biogerontology 1, 225-233.
Stefanis, L., Larsen, K. E., Rideout, H. J., Sulzer, D., and Greene, L. A. (2001). Expression of A53T mutant but not wild-type alpha-synuclein in PC12 cells induces alterations of the ubiquitin-dependent degradation system, loss of dopamine release, and autophagic cell death. Journal of Neuroscience 21, 9549-9560.
Tanaka, M., Kim, Y. M., Lee, G., Junn, E., Iwatsubo, T., and Mouradian, M. M. (2004). Aggresomes Formed by alpha-Synuclein and Synphilin-1 Are Cytoprotective. Journal of Biological Chemistry 279, 4625-4631.
Tanaka, Y., Engelender, S., Igarashi, S., Rao, R. K., Wanner, T., Tanzi, R. E., Sawa, A., Dawson, V. L., Dawson, T. M., and Ross, C. A. (2001). Inducible expression of mutant alpha-synuclein decreases proteasome activity and increases sensitivity to mitochondria-dependent apoptosis. Human Molecular Genetics 10, 919-926.
Taylor, J. P., Hardy, J., and Fischbeck, K. H. (2002). Toxic proteins in neurodegenerative disease. Science 296, 1991-1995.
Terry, R. D., Masliah, E., Salmon, D. P., Butters, N., DeTeresa, R., Hill, R., Hansen, L. A., and Katzman, R. (1991). Physical basis of cognitive alterations in Alzheimer's disease: Synapse loss is the major correlate of cognitive impairment. Annals of Neurology 30, 572-580.
Tompkins, M. M., and Hill, W. D. (1997). Contribution of somal Lewy bodies to neuronal death. Brain Research 775, 24-29.
Nocker, S., Sadis, S., Rubin, D. M., Glickman, M., Fu, H., Coux, O., Wefes, I., Finley, D., and Vierstra, R. D. (1996). The multiubiquitin-chain-binding protein Mcb1 is a component of the 26S proteasome in Saccharomyces cerevisiae and plays a nonessential, substrate-specific role in protein turnover. Molecular and Cellular Biology 16, 6020-6028.
Verhoef, L. G. G. C., Lindsten, K., Masucci, M. G., and Dantuma, N. P. (2002). Aggregate formation inhibits proteasomal degradation of polyglutamine proteins. Human Molecular Genetics 11, 2689-2700.
Verma, R., Oania, R., Graumann, J., and Deshaies, R. J. (2004). Multiubiquitin chain receptors define a layer of substrate selectivity in the ubiquitin-proteasome system. Cell 118, 99-110.
Walker, L. C., LeVine Iii, H., Mattson, M. P., and Jucker, M. (2006). Inducible proteopathies. Trends in Neurosciences 29, 438-443.
Warrick, J. M., Chan, H. Y. E., Gray-Board, G. L., Chai, Y., Paulson, H. L., and Bonini, N. M. (1999). Suppression of polyglutamine-mediated neurodegeneration in Drosophila by the molecular chaperone HSP70. Nature Genetics 23, 425-428.
Watase, K., and Zoghbi, H. Y. (2003). Modelling brain diseases in mice: The challenges of design and analysis. Nature Reviews Genetics 4, 296-307.
Wickner, R. B., Edskes, H. K., Ross, E. D., Pierce, M. M., Baxa, U., Brachmann, A., and Shewmaker, F. (2004). Prion genetics: New rules for a new kind of gene. Annual Review of Genetics 38, 681-707.
Wilkinson, C. R., Seeger, M., Hartmann-Petersen, R., Stone, M., Wallace, M., Semple, C., and Gordon, C. (2001). Proteins containing the UBA domain are able to bind to multi-ubiquitin chains. Nat Cell Biol 3, 939-943.
Yuan, J., Lipinski, M., and Degterev, A. (2003). Diversity in the mechanisms of neuronal cell death. Neuron 40, 401-413.
Zoghbi, H. Y., and Botas, J. (2002). Mouse and fly models of neurodegeneration. Trends in Genetics 18, 463-471.

### Sequences

SEQ ID NO: 1

Human ANKRD16-A
ATGGCCCAGC CCGGGGACCC GCGGCGCCTC TGCAGGCTGG
TGCAGGAGGG CCGGCTGCGC GCCCTGAAGG AGGAGCTGCA
GGCGGCCGGG GGCTGCCCGG GGCCGGCCGG GGATACCCTC
CTGCACTGCG CCGCGCGCCA CGGGCATCGG GACGTGCTGG
CCTATCTGGC CGAGGCCTGG GGCATGGACA TCGAGGCCAC
CAACCGAGAC TACAAGCGGC CTCTGCACGA GGCGGCCTCC
ATGGGCCACC GAGACTGCGT GCGCTACCTG CTGGGCCGGG
GGGCAGCGGT CGACTGCCTG AAGAAGGCCG ACTGGACTCC
TCTGATGATG GCCTGCACAA GGAAGAACCT GGGGGTGATC
CAGGAGCTGG TGGAACATGG CGCCAATCCA CTCCTGAAGA
ACAAAGATGG CTGGAACAGT TTCCACATTG CCAGTCGAGA
AGGCGACCCT CTGATCCTCC AGTACCTGCT CACTGTTTGC
CCAGGTGCCT GGAAGACAGA GAGCAAAATT AGAAGGACTC
CTCTGCATAC TGCAGCAATG CATGGCCATT TGGAGGCAGT
CAAGGTGCTT CTTAAGAGGT GCCAATATGA ACCAGACTAC
AGAGACAACT GTGGCGTCAC CGCCTTGATG GACGCAATCC
AGTGTGGGCA CATCGACGTC GCTAGGCTGC TCCTCGATGA
ACATGGGGCT TGCCTTTCAG CAGAAGACAG CCTGGGTGCC
CAGGCTCTGC ACAGGGCAGC TGTCACAGGG CAGGACGAAG
CCATCCGATT CTTGGTCTCT GAACTTGGCG TCGATGTAGA
TGTGAGAGCC ACATCAACCC ACCTCACAGC ACTTCATTAT
GCAGCTAAGG AAGGACATAC AAGTACAATT CAGACTCTCT
TATCCTTGGG AGCTGACATC AATTCTAAAG ATGAAAAAAA
TCGATCAGCC CTGCATCTGG CCTGTGCAGG TCAGCACTTG
GCCTGTGCCA AGTTTCTCCT GCAGTCGGGA CTGAAGGATT
CTGAAGACAT CACGGGCACC CTGGCTCAGC AGCTCCCAAG
GAGAGCAGAT GTCCTTCAGG GCTCTGGCCA TAGCGCAATG
ACATAA

SEQ ID NO: 2

Human ANKRD16-A
MAQPGDPRRLCRLVQEGRLRALKEELQAAGGCPGPAGDTLLHCAARHGHR
DVLAYLAEAWGMDIEATNRDYKRPLHEAASMGHRDCVRYLLGRGAAVDCL
KKADWTPLMMACTRKNLGVIQELVEHGANPLLKNKDGWNSFHIASREGDP
LILQYLLTVCPGAWKTESKIRRTPLHTAAMHGHLEAVKVLLKRCQYEPDY
RDNCGVTALMDAIQCGHIDVARLLLDEHGACLSAEDSLGAQALHRAAVTG
QDEAIRFLVSELGVDVDVRATSTHLTALHYAAKEGHTSTIQTLLSLGADI
NSKDEKNRSALHLACAGQHLACAKFLLQSGLKDSEDITGTLAQQLPRRAD
VLQGSGHSAMT

SEQ ID NO: 3

Human ANKRD16-B
ATGGCCCAGC CCGGGGACCC GCGGCGCCTC TGCAGGCTGG
TGCAGGAGGG CCGGCTGCGC GCCCTGAAGG AGGAGCTGCA
GGCGGCCGGG GGCTGCCCGG GGCCGGCCGG GGATACCCTC
CTGCACTGCG CCGCGCGCCA CGGGCATCGG GACGTGCTGG
CCTATCTGGC CGAGGCCTGG GGCATGGACA TCGAGGCCAC
CAACCGAGAC TACAAGCGGC CTCTGCACGA GGCGGCCTCC
ATGGGCCACC GAGACTGCGT GCGCTACCTG CTGGGCCGGG
GGGCAGCGGT CGACTGCCTG AAGAAGGCCG ACTGGACTCC
TCTGATGATG GCCTGCACAA GGAAGAACCT GGGGGTGATC
CAGGAGCTGG TGGAACATGG CGCCAATCCA CTCCTGAAGA
ACAAAGATGG CTGGAACAGT TTCCACATTG CCAGTCGAGA
AGGCGACCCT CTGATCCTCC AGTACCTGCT CACTGTTTGC
CCAGGTGCCT GGAAGACAGA GAGCAAAATT AGAAGGACTC
CTCTGCATAC TGCAGCAATG CATGGCCATT TGGAGGCAGT
CAAGGTGCTT CTTAAGAGGT GCCAATATGA ACCAGACTAC
AGAGACAACT GTGGCGTCAC CGCCTTGATG GACGCAATCC
AGTGTGGGCA CATCGACGTC GCTAGGCTGC TCCTCGATGA
ACATGGGGCT TGCCTTTCAG CAGAAGACAG CCTGGGTGCC
CAGGCTCTGC ACAGGGCAGC TGTCACAGGA CATACAAGTA
CAATTCAGAC TCTCTTATCC TTGGGAGCTG ACATCAATTC
TAAAGATGAA AAAAATCGAT CAGCCCTGCA TCTGGCCTGT
GCAGGTCAGC ACTTGGCCTG TGCCAAGTTT CTCCTGCAGT
CGGGACTGAA GGATTCTGAA GACATCACGG GCACCCTGGC
TCAGCAGCTC CCAAGGAGAG CAGATGTCCT TCAGGGCTCT
GGCCATAGCG CAATGACATA A

SEQ ID NO: 4

Human ANKRD16-B
MAQPGDPRRLCRLVQEGRLRALKEELQAAGGCPGPAGDTLLHCAARHGHR
DVLAYLAEAWGMDIEATNRDYKRPLHEAASMGHRDCVRYLLGRGAAVDCL
KKADWTPLMMACTRKNLGVIQELVEHGANPLLKNKDGWNSFHIASREGDP
LILQYLLTVCPGAWKTESKIRRTPLHTAAMHGHLEAVKVLLKRCQYEPDY
RDNCGVTALMDAIQCGHIDVARLLLDEHGACLSAEDSLGAQALHRAAVTG
HTSTIQTLLSLGADINSKDEKNRSALHLACAGQHLACAKFLLQSGLKDSE
DITGTLAQQLPRRADVLQGSGHSAMT

SEQ ID NO: 5

Human ANKRD16-C
ATGGCCCAGC CCGGGGACCC GCGGCGCCTC TGCAGGCTGG
TGCAGGAGGG CCGGCTGCGC GCCCTGAAGG AGGAGCTGCA
GGCGGCCGGG GGCTGCCCGG GGCCGGCCGG GGATACCCTC
CTGCACTGCG CCGCGCGCCA CGGGCATCGG GACGTGCTGG
CCTATCTGGC CGAGGCCTGG GGCATGGACA TCGAGGCCAC
CAACCGAGAC TACAAGCGGC CTCTGCACGA GGCGGCCTCC
ATGGGCCACC GAGACTGCGT GCGCTACCTG CTGGGCCGGG
GGGCAGGGGT CGACTGCCTG AAGAAGGCCG AGTGGACTCC
TCTGATGATG GCCTGCACAA GGAAGAACCT GGGGGTGATC
CAGGAGCTGG TGGAACATGG CGCCAATCCA CTCCTGAAGA
ACAAAGATGG CTGGAACAGT TTCCACATTG CCAGTCGAGA
AGGCGACCCT CTGATCCTCC AGTACCTGCT CACTGTTTGC
CCAGGTGCCT GGAAGACAGA GAGCAAAATT AGAAGGACTC
CTCTGCATAC TGCAGCAATG CATGGCCATT TGGAGGCAGT
CAAGGTGCTT CTTAAGAGGT GCCAATATGA ACCAGACTAC
AGAGACAACT GTGGCGTCAC CGCCTTGATG GACGCAATCC
AGTGTGGGCA CATCGACGTC GCTAGGCTGC TCCTCGATGA
ACATGGGGCT TGCCTTTCAG CAGAAGACAG CCTGGGTGCC
CAGGCTCTGC ACAGGGCAGC TGTCACAGGG CAGGACGAAG
CCATCCGATT CTTGGTCTCT GAACTTGGCG TCGATGTAGA
TGTGAGAGCC ACATCAACCC ACCTCACAGC ACTTCATTAT
GCAGCTAAGC CCTGCATCTG GCCTGTGCAG GTCAGCACTT
GGCCTGTGCC AAGTTTCTCC TGCAGTCGGG ACTGA

SEQ ID NO: 6

Human ANKRD16-C
MAQPGDPRRLCRLVQEGRLRALKEELQAAGGCPGPAGDTLLHCAARHGHR
DVLAYLAEAWGMDIEATNRDYKRPLHEAASMGHRDCVRYLLGRGAAVDCL
KKADWTPLMMACTRKNLGVIQELVEHGANPLLKNKDGWNSFHIASREGDP
LILQYLLTVCPGAWKTESKJRRTPLHTAAMHGHLEAVKVLLKRCQYEPDY
RDNCGVTALMDAIQCGHIDVARLLLDEHGACLSAEDSLGAQALHRAAVTG
QDEAIRFLVSELGVDVDVRATSTHLTALHYAAKPCIWPVQVSTWPVPSFS
CSRD

SEQ ID NO: 7

GTCACAGGA CATACAAGT

SEQ ID NO: 8

VTGHTS

SEQ ID NO: 9

C CCTGCATCTG GCCTGTGCAG GTCAGCACTT GGCCTGTGCC
AAGTTTCTCC TGCAGTCGGG ACTGA

SEQ ID NO: 10

PCIWPVQVSTWPVPSFSCSRD

SEQ ID NO: 11

GCTAAGG AAGGACATAC A

SEQ ID NO: 12

AKEGHT

SEQ ID NO: 13

ATGGCCCAGC CCGGGGACCC GCGGCGCCTC TGCAGGCTGG
TGCAGGAGGG CCGGCTGCGC GCCCTGAAGG AGGAGCTGCA
GGCGGCCGGG GGCTGCCCGG GGCCGGCCGG GGATACCCTC
CTGCACTGCG CCGCGCGCCA CGGGCATCGG GACGTGCTGG
CCTATCTGGC CGAGGCCTGG GGCATGGACA TCGAGGCCAC
CAACCGAGAC TACAAGCGGC CTCTGCACGA GGCGGCCTCC
ATGGGCCACC GAGACTGCGT GCGCTACCTG CTGGGCCGGG
GGGCAGCGGT CGACTGCCTG AAGAAGGCCG ACTGGACTCC
TCTGATGATG GCCTGCACAA GGAAGAACCT GGGGGTGATC
CAGGAGCTGG TGGAACATGG CGCCAATCCA CTCCTGAAGA
ACAAAGATGG CTGGAACAGT TTCCACATTG CCAGTCGAGA
AGGCGACCCT CTGATCCTCC AGTACCTGCT CACTGTTTGC
CCAGGTGCCT GGAAGACAGA GAGCAAAATT AGAAGGACTC
CTCTGCATAC TGCAGCAATG CATGGCCATT TGGAGGCAGT
CAAGGTGCTT CTTAAGAGGT GCCAATATGA ACCAGACTAC
AGAGACAACT GTGGCGTCAC CGCCTTGATG GACGCAATCC
AGTGTGGGCA CATCGACGTC GCTAGGCTGC TCCTCGATGA
ACATGGGGCT TGCCTTTCAG CAGAAGACAG CCTGGGTGCC
CAGGCTCTGC ACAGGGCAGC TGTCACAGGG

SEQ ID NO: 14

MAQPGDPRRLCRLVQEGRLRALKEELQAAGGCPGPAGDTLLHCAARHGHR
DVLAYLAEAWGMDIEATNRDYKRPLHEAASMGHRDCVRYLLGRGAAVDCL
KKADWTPLMMACTRKNLGVIQELVEHGANPLLKNKDGWNSFHIASREGDP
LILQYLLTVCPGAWKTESKIRRTPLHTAAMHGHLEAVKVLLKRCQYEPDY
RDNCGVTALMDAIQCGHIDVARLLLDEHGACLSAEDSLGAQALHRAAVTG

SEQ ID NO: 15

Mouse ANKRD16-A
ATGGCTCTGC CTGGGGATCC GCGGCGCCTC TGCAGGCTGG
TGCAAGAGGG CCGACTGCGT GACCTTCAGG AGGAACTGGC
GGTAGCTAGA GGTTGCCGGG GGCCAGCCGG AGACACCCTT
CTCCACTGTG CAGCACGCCA CGGACGCCAG GATATCCTAG
CGTACCTAGT GGAGGCTTGG AGTATGGACA TCGAGGCTAC
CAACCGAGAC TACAAGCGGC CTCTGCACGA AGCTGCCTCT
ATGGGCCACC GGGACTGCGT GCGCTACCTC CTGGGCCGAG
GTGCAGTCGT GGACTCCTTG AAGAAGGCGG ACTGGACTCC
TCTGATGATG GCGTGCACAA GGAAGAACCT TGATGTGATC
CAGGACCTTG TAGAACACGG TGCCAATCCA CTCCTGAAGA
ACAAGGATGG CTGGAACAGT TTCCACATTG CCAGTAGAGA
AGGCCACCCT GTGATCCTCC GCAATGCACG GCTGTTTGGA
AGCAGTCCAG GTGCTTCTTG A

SEQ ID NO: 16

Mouse ANKRD16-A
MALPGDPRRLCRLVQEGRLRDLQEELAVARGCRGPAGDTLLHCAARHGRQ
DILAYLVEAWSMDIEATNRDYKRPLHEAASMGHRDCVRYLLGRGAVVDSL
KKADWTPLMMACTRKNLDVIQDLVEHGANPLLKNKDGWNSFHIASREGHP
VILRNARLFGSSPGAS

SEQ ID NO: 17

Mouse ANKRD16-B
ATGGCTCTGC CTGGGGATCC GCGGCGCCTC TGCAGGCTGG
TGCAAGAGGG CCGACTGCGT GACCTTCAGG AGGAACTGGC
GGTAGCTAGA GGTTGCCGGG GGCCAGCCGG AGACACCCTT
CTCCACTGTG CAGCACGCCA CGGACGCCAG GATATCCTAG
CGTACCTAGT GGAGGCTTGG AGTATGGACA TCGAGGCTAC
CAACCGAGAC TACAAGCGGC CTCTGCACGA AGCTGCCTCT
ATGGGCCACC GGGACTGCGT GCGCTACCTC CTGGGCCGAG
GTGCAGTCGT GGACTCCTTG AAGAAGGCGG ACTGGACTCC
TCTGATGATG GCGTGCACAA GGAAGAACCT TGATGTGATC
CAGGACCTTG TAGAACACGG TGCCAATCCA CTCCTGAAGA
ACAAGGATGG CTGGAACAGT TTCCACATTG CCAGTAGAGA
AGGCCACCCT GTGATCCTCC GGTACTTGCT CACTGTCTGC
CCTGATGCTT GGAAAACAGA GAGCAACATT AGAAGAACCC
CTTTACACAC TGCAGCAATG CACGGCTGTT TGGAAGCAGT
CCAGGTGCTT CTTGAAAGGT GTCACTATGA ACCAGACTGT
CGAGACAACT GTGGTGTCAC GCCCTTCATG GATGCAATTC
AGTGTGGCCA CGTTAGTATA GCCAAGCTGC TCCTTGAACA
GCATAAGGTA TAAGGCTTGC TCTTCAGCTG CAGATAG

SEQ ID NO: 18

Mouse ANKRD16-B
MALPGDPRRLCRLVQEGRLRDLQEELAVARGCRGPAGDTLLHCAARHGRQ
DILAYLVEAWSMDIEATNRDYKRPLHEAASMGHRDCVRYLLGRGAVVDSL
KKADWTPLMMACTRKNLDVIQDLVEHGANPLLKNKDGWNSFHIASREGHP
VILRYLLTVCPDAWKTESNIRRTPLHTAAMHGCLEAVQVLLERCHYEPDC
RDNCGVTPFMDAIQCGHVSIAKLLLEQHKV

SEQ ID NO: 19

Mouse ANKRD16-C
ATGGCTCTGC CTGGGGATCC GCGGCGCCTC TGCAGGCTGG
TGCAAGAGGG CCGACTGCGT GACCTTCAGG AGGAACTGGC
GGTAGCTAGA GGTTGCCGGG GGCCAGCCGG AGACACCCTT
CTCCACTGTG CAGCACGCCA CGGACGCCAG GATATCCTAG
CGTACCTAGT GGAGGCTTGG AGTATGGACA TCGAGGCTAC
CAACCGAGAC TACAAGCGGC CTCTGCACGA AGCTGCCTCT
ATGGGCCACC GGGACTGCGT GCGCTACCTC CTGGGCCGAG
GTGCAGTCGT GGACTCCTTG AAGAAGGCGG ACTGGACTCC
TCTGATGATG GCGTGCACAA GGAAGAACCT TGATGTGATC
CAGGACCTTG TAGAACACGG TGCCAATCCA CTCCTGAAGA
ACAAGGATGG CTGGAACAGT TTCCACATTG CCAGTAGAGA
AGGCCACCCT GTGATCCTCC GGTACTTGCT CACTGTCTGC
CCTGATGCTT GGAAAACAGA GAGCAACATT AGAAGAACCC
CTTTACACAC TGCAGCAATG CACGGCTGTT TGGAAGCAGT
CCAGGTGCTT CTTGAAAGGT GTCACTATGA ACCAGACTGT
CGAGACAACT GTGGTGTCAC GCCCTTCATG GATGCAATTC
AGTGTGGCCA CGTTAGTATA GCCAAGCTGC TCCTTGAACA
GCATAAGGCT TGCTCTTCAG CTGCAGATAG CATGGGGGCC
CAGGCTCTAC ACCGCGCAGC AGTCACTGGG CAGGATGAAG
CCATACGGTT CCTGGTATGC GGTCTTGGCA TCGATGTAGA
TGTAAGAGCA AAGTCAAGCC AGCTCACAGC ACTTCACTAT
GCAGCAAGAG TTCCAACACC CACATCAGGC AACTCACAAC
TATCTATAAC TCCAGCTCCA GAGGATCTGT GGTGTCACAC
CCTTCATGGA TACACTTCAG TTCAGCCACG TTTGCATAG

SEQ ID NO: 20

Mouse ANKRD16-C
MALPGDPRRLCRLVQEGRLRDLQEELAVARGCRGPAGDTLLHCAARHGRQ
DILAYLVEAWSMDIEATNRDYKRPLHEAASMGHRDCVRYLLGRGAVVDSL
KKADWTPLMMACTRKNLDVIQDLVEHGANPLLKNKDGWNSFHIASREGHP
VILRYLLTVCPDAWKTESNIRRTPLHTAAMHGCLEAVQVLLERCHYEPDC
RDNCGVTPFMDAIQCGHVSIAKLLLEQHKACSSAADSMGAQALHRAAVTG
QDEAIRFLVCGLGIDVDVRAKSSQLTALHYAAKSSNTHIRQLTTIYNSSS
RGSVVSHPSWIHFSSATFA

SEQ ID NO: 21

Mouse ANKRD16-D
ATGGCTCTGC CTGGGGATCC GCGGCGCCTC TGCAGGCTGG
TGCAAGAGGG CCGACTGCGT GACCTTCAGG AGGAACTGGC
GGTAGCTAGA GGTTGCCGGG GGCCAGCCGG AGACACCCTT
CTCCACTGTG CAGCACGCCA CGGACGCCAG GATATCCTAG
CGTACCTAGT GGAGGCTTGG AGTATGGACA TCGAGGCTAC
CAACCGAGAC TACAAGCGGC CTCTGCACGA AGCTGCCTCT
ATGGGCCACC GGGACTGCGT GCGCTACCTC CTGGGCCGAG
GTGCAGTCGT GGACTCCTTG AAGAAGGCGG ACTGGACTCC
TCTGATGATG GCGTGCACAA GGAAGAACCT TGATGTGATC
CAGGACCTTG TAGAACACGG TGCCAATCCA CTCCTGAAGA
ACAAGGATGG CTGGAACAGT TTCCACATTG CCAGTAGAGA
AGGCCACCCT GTGATCCTCC GGTACTTGCT CACTGTCTGC
CCTGATGCTT GGAAAACAGA GAGCAACATT AGAAGAACCC
CTTTACACAC TGCAGCAATG CACGGCTGTT TGGAAGCAGT
CCAGGTGCTT CTTGAAAGGT GTCACTATGA ACCAGACTGT
CGAGACAACT GTGGTGTCAC GCCCTTCATG GATGCAATTC
AGTGTGGCCA CGTTAGTATA GCCAAGCTGC TCCTTGAACA
GCATAAGGCT TGCTCTTCAG CTGCAGATAG CATGGGGGCC
CAGGCTCTAC ACCGCGCAGC AGTCACTGGG CAGGATGAAG
CCATACGGTT CCTGGTATGC GGTCTTGGCA TCGATGTAGA
TGTAAGAGCA AAGTCAAGCC AGCTCACAGC ACTTCACTAT
GCAGCAAGGA AGGACAGACG AATACAGTTC AAACTCTGTT
GTCCTTGGGT GCCGACATCA ACTCTACAGA TGAAAGAAAT
CGCTCAGTCC TGCATCTGGC CTGCGCAGGT CAGCATGTGG
CTTGCACCAG GCTCCTCCTA CAGTCGGGAC TGAAGGATTC
CGAAGACCTC ACAGGCACCT TGGCCCAGCA GCTCACGAGA
AGCGTAGATA TCCTTCAGGA CTTTGACCAT GACGTGAAAT
CGTAG

SEQ ID NO: 22

Mouse ANKRD16-D
MALPGDPRRLCRLVQEGRLRDLQEELAVARGCRGPAGDTLLHCAARHGRQ
DILAYLVEAWSMDIEATNRDYKRPLHEAASMGHRDCVRYLLGRGAVVDSL
KKADWTPLMMACTRKNLDVIQDLVEHGANPLLKNKDGWNSFHIASREGHP
VILRYLLTVCPDAWKTESNIRRTPLHTAAMHGCLEAVQVLLERCHYEPDC
RDNCGVTPFMDAIQCGHVSIAKLLLEQHKACSSAADSMGAQALHRAAVTG
QDEAIRFLVCGLGIDVDVRAKSSQLTALHYAAKEGQTNTVQTLLSLGADI
NSTDERNRSVLHLACAGQHVACTRLLLQSGLKDSADLTGTLAQQLMRSVD
ILQDFDHDVKS

SEQ ID NO: 23

AVTGHTST

SEQ ID NO: 24

AAVTGHTSTI

SEQ ID NO: 25

AAKEGHTS

SEQ ID NO: 26

YAAKEGHTST

1-115. (canceled)

116. A method of treating a neurodegenerative disease or a proteopathy, the method comprising administering to a subject in need thereof an effective amount of a composition comprising ANKRD16 protein or a nucleic acid encoding ANKRD16.

117. The method of claim 1, wherein the ANKRD16 protein is selected from SEQ ID Nos: 2, 4, 6, 8, 10, 12, 14 or 22.

118. The method of claim 1, wherein a neurodegenerative disease is selected from the group consisting of: Alexander disease, Alper's disease, Alzheimer's disease, Amyotrophic lateral sclerosis, Ataxia telangiectasia, Batten disease, Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease, Multiple sclerosis, Multiple System Atrophy, Parkinson disease, Pelizaeus-Merzbacher Disease, Pick's disease, Primary lateral sclerosis, Prion diseases, Refsum's disease, Sandhoff disease, Schilder's disease, Schizophrenia, Spielmeyer-Vogt-Sjogren-Batten disease, Spinocerebellar ataxia, Spinal muscular atrophy, Steele-Richardson-Olszewski disease, and Tabes dorsalis.

119. The method of claim 1, wherein the proteopathy is selected from the group consisting of: infertility, reduced fertility, cancer, Hereditary lattice corneal dystrophy, cataracts, myopathy, amyloidosis, diabetes, medullary thyroid carcinoma, Pituitary prolactinoma, and Pulmonary alveolar proteinosis.

120. The method of claim 1, wherein the said composition is administered systemically or locally.

121. The method of claim 1, wherein said subject is a human.

122. The method of claim 1, wherein said composition promotes neuronal cell survival.

123. The method of claim 1, wherein said composition is formulated with a pharmaceutically acceptable carrier.

124. The method of claim 1, further comprising at least one additional therapeutic for a neurodegenerative disease or a proteopathy.

125. A method of detecting whether a subject has or is at risk of developing a neurodegenerative disease or a proteopathy, comprising:
(a) contacting a sample obtained from said subject with at least one probe that binds at least one ANKRD16 isoform; and
(b) assessing the presence of full-length and short ANKRD16 isoforms, wherein the presence of higher amounts of short ANKRD16 isoforms compared to the full-length ANKRD16 isoform is indicative that the subject has or is at risk of developing a neurodegenerative disease or a proteopathy.

126. The method of claim 125, wherein said probe is selected from the group consisting of an antibody or antigen binding fragment thereof, a non-immunoglobulin antigen-binding scaffold, and a nucleic acid.

127. The method of claim 125, wherein said isoform is selected from the group consisting of protein or RNA isoforms.

128. The method of claim 125, wherein said subject is a human.

129. The method of claim 125, wherein said neurodegenerative disease is selected from the group consisting of: Alexander disease, Alper's disease, Alzheimer's disease, Amyotrophic lateral sclerosis, Ataxia telangiectasia, Batten disease, Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease, Multiple sclerosis, Multiple System Atrophy, Parkinson disease, Pelizaeus-Merzbacher Disease, Pick's disease, Primary lateral sclerosis, Prion diseases, Refsum's disease, Sandhoff disease, Schilder's disease, Schizophrenia, Spielmeyer-Vogt-Sjogren-Batten disease, Spinocerebellar ataxia, Spinal muscular atrophy, Steele-Richardson-Olszewski disease, or Tabes dorsalis.

130. The method of claim 125, wherein the proteopathy is selected from the group consisting of: infertility, reduced fertility, cancer, Hereditary lattice corneal dystrophy, cataracts, myopathy, amyloidosis, diabetes, medullary thyroid carcinoma, Pituitary prolactinoma, and Pulmonary alveolar proteinosis.

131. A kit for diagnosis or prognosis of a neurodegenerative disease or a proteopathy comprising at least one probe that binds at least one ANKRD16 isoform, a detectable label, and instructions for using the kit, wherein said probe is selected from the group consisting of an antibody or antigen binding fragment thereof or a nucleic acid.

132. A device for detecting whether a subject has or is at risk of developing a neurodegenerative disease or proteopathy comprising a solid matrix and an ANKRD16 probe deposited on said solid support matrix, wherein said probe facilitates the measurement of the level of ANKRD16 in a test sample.

133. The device of claim 132, wherein the ANKRD16 probe is an antibody or antigen binding fragment or non-immunoglobulin antigen-binding scaffold, which specifically binds ANKRD16 protein to form an antibody or antigen binding-ANKRD16 protein complex.

134. The device of claim 132, wherein the solid matrix is a dipstick, bead, or a microtiter plate.


SEQUENCE LISTING (29 sequences)

SEQ ID NO: 1 - DNA, 1086 bp, Homo sapiens
atggcccagcccggggacccgcggcgcctctgcaggctggtgcaggagggccggctgcgcgccctgaaggaggagctgcaggcggccgggggctgcccggggccggccggggataccctcctgcactgcgccgcgcgccacgggcatcgggacgtgctggcctatctggccgaggcctggggcatggacatcgaggccaccaaccgagactacaagcggcctctgcacgaggcggcctccatgggccaccgagactgcgtgcgctacctgctgggccggggggcagcggtcgactgcctgaagaaggccgactggactcctctgatgatggcctgcacaaggaagaacctgggggtgatccaggagctggtggaacatggcgccaatccactcctgaagaacaaagatggctggaacagtttccacattgccagtcgagaaggcgaccctctgatcctccagtacctgctcactgtttgcccaggtgcctggaagacagagagcaaaattagaaggactcctctgcatactgcagcaatgcatggccatttggaggcagtcaaggtgcttcttaagaggtgccaatatgaaccagactacagagacaactgtggcgtcaccgccttgatggacgcaatccagtgtgggcacatcgacgtcgctaggctgctcctcgatgaacatggggcttgcctttcagcagaagacagcctgggtgcccaggctctgcacagggcagctgtcacagggcaggacgaagccatccgattcttggtctctgaacttggcgtcgatgtagatgtgagagccacatcaacccacctcacagcacttcattatgcagctaaggaaggacatacaagtacaattcagactctcttatccttgggagctgacatcaattctaaagatgaaaaaaatcgatcagccctgcatctggcctgtgcaggtcagcacttggcctgtgccaagtttctcctgcagtcgggactgaaggattctgaagacatcacgggcaccctggctcagcagctcccaaggagagcagatgtccttcagggctctggccatagcgcaatgacataa

SEQ ID NO: 2 - protein, 361 aa, Homo sapiens
MAQPGDPRRLCRLVQEGRLRALKEELQAAGGCPGPAGDTLLHCAARHGHRDVLAYLAEAWGMDIEATNRDYKRPLHEAASMGHRDCVRYLLGRGAAVDCLKKADWTPLMMACTRKNLGVIQELVEHGANPLLKNKDGWNSFHIASREGDPLILQYLLTVCPGAWKTESKIRRTPLHTAAMHGHLEAVKVLLKRCQYEPDYRDNCGVTALMDAIQCGHIDVARLLLDEHGACLSAEDSLGAQALHRAAVTGQDEAIRFLVSELGVDVDVRATSTHLTALHYAAKEGHTSTIQTLLSLGADINSKDEKNRSALHLACAGQHLACAKFLLQSGLKDSEDITGTLAQQLPRRADVLQGSGHSAMT

SEQ ID NO: 3 - DNA, 981 bp, Homo sapiens
atggcccagcccggggacccgcggcgcctctgcaggctggtgcaggagggccggctgcgcgccctgaaggaggagctgcaggcggccgggggctgcccggggccggccggggataccctcctgcactgcgccgcgcgccacgggcatcgggacgtgctggcctatctggccgaggcctggggcatggacatcgaggccaccaaccgagactacaagcggcctctgcacgaggcggcctccatgggccaccgagactgcgtgcgctacctgctgggccggggggcagcggtcgactgcctgaagaaggccgactggactcctctgatgatggcctgcacaaggaagaacctgggggtgatccaggagctggtggaacatggcgccaatccactcctgaagaacaaagatggctggaacagtttccacattgccagtcgagaaggcgaccctctgatcctccagtacctgctcactgtttgcccaggtgcctggaagacagagagcaaaattagaaggactcctctgcatactgcagcaatgcatggccatttggaggcagtcaaggtgcttcttaagaggtgccaatatgaaccagactacagagacaactgtggcgtcaccgccttgatggacgcaatccagtgtgggcacatcgacgtcgctaggctgctcctcgatgaacatggggcttgcctttcagcagaagacagcctgggtgcccaggctctgcacagggcagctgtcacaggacatacaagtacaattcagactctcttatccttgggagctgacatcaattctaaagatgaaaaaaatcgatcagccctgcatctggcctgtgcaggtcagcacttggcctgtgccaagtttctcctgcagtcgggactgaaggattctgaagacatcacgggcaccctggctcagcagctcccaaggagagcagatgtccttcagggctctggccatagcgcaatgacataa

SEQ ID NO: 4 - protein, 326 aa, Homo sapiens
MAQPGDPRRLCRLVQEGRLRALKEELQAAGGCPGPAGDTLLHCAARHGHRDVLAYLAEAWGMDIEATNRDYKRPLHEAASMGHRDCVRYLLGRGAAVDCLKKADWTPLMMACTRKNLGVIQELVEHGANPLLKNKDGWNSFHIASREGDPLILQYLLTVCPGAWKTESKIRRTPLHTAAMHGHLEAVKVLLKRCQYEPDYRDNCGVTALMDAIQCGHIDVARLLLDEHGACLSAEDSLGAQALHRAAVTGHTSTIQTLLSLGADINSKDEKNRSALHLACAGQHLACAKFLLQSGLKDSEDITGTLAQQLPRRADVLQGSGHSAMT

SEQ ID NO: 5 - DNA, 915 bp, Homo sapiens
atggcccagcccggggacccgcggcgcctctgcaggctggtgcaggagggccggctgcgcgccctgaaggaggagctgcaggcggccgggggctgcccggggccggccggggataccctcctgcactgcgccgcgcgccacgggcatcgggacgtgctggcctatctggccgaggcctggggcatggacatcgaggccaccaaccgagactacaagcggcctctgcacgaggcggcctccatgggccaccgagactgcgtgcgctacctgctgggccggggggcagcggtcgactgcctgaagaaggccgactggactcctctgatgatggcctgcacaaggaagaacctgggggtgatccaggagctggtggaacatggcgccaatccactcctgaagaacaaagatggctggaacagtttccacattgccagtcgagaaggcgaccctctgatcctccagtacctgctcactgtttgcccaggtgcctggaagacagagagcaaaattagaaggactcctctgcatactgcagcaatgcatggccatttggaggcagtcaaggtgcttcttaagaggtgccaatatgaaccagactacagagacaactgtggcgtcaccgccttgatggacgcaatccagtgtgggcacatcgacgtcgctaggctgctcctcgatgaacatggggcttgcctttcagcagaagacagcctgggtgcccaggctctgcacagggcagctgtcacagggcaggacgaagccatccgattcttggtctctgaacttggcgtcgatgtagatgtgagagccacatcaacccacctcacagcacttcattatgcagctaagccctgcatctggcctgtgcaggtcagcacttggcctgtgccaagtttctcctgcagtcgggactga

SEQ ID NO: 6 - protein, 304 aa, Homo sapiens
MAQPGDPRRLCRLVQEGRLRALKEELQAAGGCPGPAGDTLLHCAARHGHRDVLAYLAEAWGMDIEATNRDYKRPLHEAASMGHRDCVRYLLGRGAAVDCLKKADWTPLMMACTRKNLGVIQELVEHGANPLLKNKDGWNSFHIASREGDPLILQYLLTVCPGAWKTESKIRRTPLHTAAMHGHLEAVKVLLKRCQYEPDYRDNCGVTALMDAIQCGHIDVARLLLDEHGACLSAEDSLGAQALHRAAVTGQDEAIRFLVSELGVDVDVRATSTHLTALHYAAKPCIWPVQVSTWPVPSFSCSRD

SEQ ID NO: 7 - DNA, 18 bp, Artificial Sequence
  Description of Artificial Sequence Synthetic oligonucleotide
gtcacaggacatacaagt

SEQ ID NO: 8 - protein, 6 aa, Artificial Sequence
  Description of Artificial Sequence Synthetic peptide
VTGHTS

SEQ ID NO: 9 - DNA, 66 bp, Artificial Sequence
  Description of Artificial Sequence Synthetic oligonucleotide
ccctgcatctggcctgtgcaggtcagcacttggcctgtgccaagtttctcctgcagtcgggactga

SEQ ID NO: 10 - protein, 21 aa, Artificial Sequence
  Description of Artificial Sequence Synthetic peptide
PCIWPVQVSTWPVPSFSCSRD

SEQ ID NO: 11 - DNA, 18 bp, Artificial Sequence
  Description of Artificial Sequence Synthetic oligonucleotide
gctaaggaaggacataca

SEQ ID NO: 12 - protein, 6 aa, Artificial Sequence
  Description of Artificial Sequence Synthetic peptide
AKEGHT

SEQ ID NO: 13 - DNA, 750 bp, Homo sapiens
atggcccagcccggggacccgcggcgcctctgcaggctggtgcaggagggccggctgcgcgccctgaaggaggagctgcaggcggccgggggctgcccggggccggccggggataccctcctgcactgcgccgcgcgccacgggcatcgggacgtgctggcctatctggccgaggcctggggcatggacatcgaggccaccaaccgagactacaagcggcctctgcacgaggcggcctccatgggccaccgagactgcgtgcgctacctgctgggccggggggcagcggtcgactgcctgaagaaggccgactggactcctctgatgatggcctgcacaaggaagaacctgggggtgatccaggagctggtggaacatggcgccaatccactcctgaagaacaaagatggctggaacagtttccacattgccagtcgagaaggcgaccctctgatcctccagtacctgctcactgtttgcccaggtgcctggaagacagagagcaaaattagaaggactcctctgcatactgcagcaatgcatggccatttggaggcagtcaaggtgcttcttaagaggtgccaatatgaaccagactacagagacaactgtggcgtcaccgccttgatggacgcaatccagtgtgggcacatcgacgtcgctaggctgctcctcgatgaacatggggcttgcctttcagcagaagacagcctgggtgcccaggctctgcacagggcagctgtcacaggg

SEQ ID NO: 14 - protein, 250 aa, Homo sapiens
MAQPGDPRRLCRLVQEGRLRALKEELQAAGGCPGPAGDTLLHCAARHGHRDVLAYLAEAWGMDIEATNRDYKRPLHEAASMGHRDCVRYLLGRGAAVDCLKKADWTPLMMACTRKNLGVIQELVEHGANPLLKNKDGWNSFHIASREGDPLILQYLLTVCPGAWKTESKIRRTPLHTAAMHGHLEAVKVLLKRCQYEPDYRDNCGVTALMDAIQCGHIDVARLLLDEHGACLSAEDSLGAQALHRAAVTG

SEQ ID NO: 15 - DNA, 501 bp, Mus musculus
atggctctgcctggggatccgcggcgcctctgcaggctggtgcaagagggccgactgcgtgaccttcaggaggaactggcggtagctagaggttgccgggggccagccggagacacccttctccactgtgcagcacgccacggacgccaggatatcctagcgtacctagtggaggcttggagtatggacatcgaggctaccaaccgagactacaagcggcctctgcacgaagctgcctctatgggccaccgggactgcgtgcgctacctcctgggccgaggtgcagtcgtggactccttgaagaaggcggactggactcctctgatgatggcgtgcacaaggaagaaccttgatgtgatccaggaccttgtagaacacggtgccaatccactcctgaagaacaaggatggctggaacagtttccacattgccagtagagaaggccaccctgtgatcctccgcaatgcacggctgtttggaagcagtccaggtgcttcttga

SEQ ID NO: 16 - protein, 166 aa, Mus musculus
MALPGDPRRLCRLVQEGRLRDLQEELAVARGCRGPAGDTLLHCAARHGRQDILAYLVEAWSMDIEATNRDYKRPLHEAASMGHRDCVRYLLGRGAVVDSLKKADWTPLMMACTRKNLDVIQDLVEHGANPLLKNKDGWNSFHIASREGHPVILRNARLFGSSPGAS

SEQ ID NO: 17 - DNA, 717 bp, Mus musculus
atggctctgcctggggatccgcggcgcctctgcaggctggtgcaagagggccgactgcgtgaccttcaggaggaactggcggtagctagaggttgccgggggccagccggagacacccttctccactgtgcagcacgccacggacgccaggatatcctagcgtacctagtggaggcttggagtatggacatcgaggctaccaaccgagactacaagcggcctctgcacgaagctgcctctatgggccaccgggactgcgtgcgctacctcctgggccgaggtgcagtcgtggactccttgaagaaggcggactggactcctctgatgatggcgtgcacaaggaagaaccttgatgtgatccaggaccttgtagaacacggtgccaatccactcctgaagaacaaggatggctggaacagtttccacattgccagtagagaaggccaccctgtgatcctccggtacttgctcactgtctgccctgatgcttggaaaacagagagcaacattagaagaacccctttacacactgcagcaatgcacggctgtttggaagcagtccaggtgcttcttgaaaggtgtcactatgaaccagactgtcgagacaactgtggtgtcacgcccttcatggatgcaattcagtgtggccacgttagtatagccaagctgctccttgaacagcataaggtataaggcttgctcttcagctgcagatag

SEQ ID NO: 18 - protein, 230 aa, Mus musculus
MALPGDPRRLCRLVQEGRLRDLQEELAVARGCRGPAGDTLLHCAARHGRQDILAYLVEAWSMDIEATNRDYKRPLHEAASMGHRDCVRYLLGRGAVVDSLKKADWTPLMMACTRKNLDVIQDLVEHGANPLLKNKDGWNSFHIASREGHPVILRYLLTVCPDAWKTESNIRRTPLHTAAMHGCLEAVQVLLERCHYEPDCRDNCGVTPFMDAIQCGHVSIAKLLLEQHKV

SEQ ID NO: 19 - DNA, 959 bp, Mus musculus
atggctctgcctggggatccgcggcgcctctgcaggctggtgcaagagggccgactgcgtgaccttcaggaggaactggcggtagctagaggttgccgggggccagccggagacacccttctccactgtgcagcacgccacggacgccaggatatcctagcgtacctagtggaggcttggagtatggacatcgaggctaccaaccgagactacaagcggcctctgcacgaagctgcctctatgggccaccgggactgcgtgcgctacctcctgggccgaggtgcagtcgtggactccttgaagaaggcggactggactcctctgatgatggcgtgcacaaggaagaaccttgatgtgatccaggaccttgtagaacacggtgccaatccactcctgaagaacaaggatggctggaacagtttccacattgccagtagagaaggccaccctgtgatcctccggtacttgctcactgtctgccctgatgcttggaaaacagagagcaacattagaagaacccctttacacactgcagcaatgcacggctgtttggaagcagtccaggtgcttcttgaaaggtgtcactatgaaccagactgtcgagacaactgtggtgtcacgcccttcatggatgcaattcagtgtggccacgttagtatagccaagctgctccttgaacagcataaggcttgctcttcagctgcagatagcatgggggcccaggctctacaccgcgcagcagtcactgggcaggatgaagccatacggttcctggtatgcggtcttggcatcgatgtagatgtaagagcaaagtcaagccagctcacagcacttcactatgcagcaagagttccaacacccacatcaggcaactcacaactatctataactccagctccagaggatctgtggtgtcacacccttcatggatacacttcagttcagccacgtttgcatag

SEQ ID NO: 20 - protein, 319 aa, Mus musculus
MALPGDPRRLCRLVQEGRLRDLQEELAVARGCRGPAGDTLLHCAARHGRQDILAYLVEAWSMDIEATNRDYKRPLHEAASMGHRDCVRYLLGRGAVVDSLKKADWTPLMMACTRKNLDVIQDLVEHGANPLLKNKDGWNSFHIASREGHPVILRYLLTVCPDAWKTESNIRRTPLHTAAMHGCLEAVQVLLERCHYEPDCRDNCGVTPFMDAIQCGHVSIAKLLLEQHKACSSAADSMGAQALHRAAVTGQDEAIRFLVCGLGIDVDVRAKSSQLTALHYAAKSSNTHIRQLTTIYNSSSRGSVVSHPSWIHFSSATFA

SEQ ID NO: 21 - DNA, 1085 bp, Mus musculus
atggctctgcctggggatccgcggcgcctctgcaggctggtgcaagagggccgactgcgtgaccttcaggaggaactggcggtagctagaggttgccgggggccagccggagacacccttctccactgtgcagcacgccacggacgccaggatatcctagcgtacctagtggaggcttggagtatggacatcgaggctaccaaccgagactacaagcggcctctgcacgaagctgcctctatgggccaccgggactgcgtgcgctacctcctgggccgaggtgcagtcgtggactccttgaagaaggcggactggactcctctgatgatggcgtgcacaaggaagaaccttgatgtgatccaggaccttgtagaacacggtgccaatccactcctgaagaacaaggatggctggaacagtttccacattgccagtagagaaggccaccctgtgatcctccggtacttgctcactgtctgccctgatgcttggaaaacagagagcaacattagaagaacccctttacacactgcagcaatgcacggctgtttggaagcagtccaggtgcttcttgaaaggtgtcactatgaaccagactgtcgagacaactgtggtgtcacgcccttcatggatgcaattcagtgtggccacgttagtatagccaagctgctccttgaacagcataaggcttgctcttcagctgcagatagcatgggggcccaggctctacaccgcgcagcagtcactgggcaggatgaagccatacggttcctggtatgcggtcttggcatcgatgtagatgtaagagcaaagtcaagccagctcacagcacttcactatgcagcaaggaaggacagacgaatacagttcaaactctgttgtccttgggtgccgacatcaactctacagatgaaagaaatcgctcagtcctgcatctggcctgcgcaggtcagcatgtggcttgcaccaggctcctcctacagtcgggactgaaggattccgaagacctcacaggcaccttggcccagcagctcacgagaagcgtagatatccttcaggactttgaccatgacgtgaaatcgtag

SEQ ID NO: 22 - protein, 361 aa, Mus musculus
MALPGDPRRLCRLVQEGRLRDLQEELAVARGCRGPAGDTLLHCAARHGRQDILAYLVEAWSMDIEATNRDYKRPLHEAASMGHRDCVRYLLGRGAVVDSLKKADWTPLMMACTRKNLDVIQDLVEHGANPLLKNKDGWNSFHIASREGHPVILRYLLTVCPDAWKTESNIRRTPLHTAAMHGCLEAVQVLLERCHYEPDCRDNCGVTPFMDAIQCGHVSIAKLLLEQHKACSSAADSMGAQALHRAAVTGQDEAIRFLVCGLGIDVDVRAKSSQLTALHYAAKEGQTNTVQTLLSLGADINSTDERNRSVLHLACAGQHVACTRLLLQSGLKDSADLTGTLAQQLMRSVDILQDFDHDVKS

SEQ ID NO: 23 - protein, 8 aa, Artificial Sequence
  Description of Artificial Sequence Synthetic peptide
AVTGHTST

SEQ ID NO: 24 - protein, 10 aa, Artificial Sequence
  Description of Artificial Sequence Synthetic peptide
AAVTGHTSTI

SEQ ID NO: 25 - protein, 8 aa, Artificial Sequence
  Description of Artificial Sequence Synthetic peptide
AAKEGHTS

SEQ ID NO: 26 - protein, 10 aa, Artificial Sequence
  Description of Artificial Sequence Synthetic peptide
YAAKEGHTST

SEQ ID NO: 27 - DNA, 20 bp, Artificial Sequence
  Description of Artificial Sequence Synthetic oligonucleotide
tcttgcagagttccaacacc

SEQ ID NO: 28 - DNA, 20 bp, Artificial Sequence
  Description of Artificial Sequence Synthetic oligonucleotide
tggaaaacatgaggtataaa

SEQ ID NO: 29 - DNA, 20 bp, Artificial Sequence
  Description of Artificial Sequence Synthetic oligonucleotide
tggaaaacatggggtataaa